(19) United States
(12) Patent Application Publication
(10) Pub. No.: US 2007/0066552 A1
(43) Pub. Date: Mar. 22, 2007

(54) Topical administration permitting prolonged exposure of target cells to therapeutic and prophylactic nucleic acids

(75) Inventors: Peter Clarke, Sugar Land, TX (US); Sunil Chada, Missouri City, TX (US); Kerstin Menander, Bellaire, TX (US); Robert E. Sobol, Rancho Santa Fe, CA (US); Shuyuan Zhang, Sugar Land, TX (US)

Correspondence Address: FULBRIGHT & JAWORSKI L.L.P., 600 CONGRESS AVE., SUITE 2400, AUSTIN, TX, 78701, US

(73) Assignee: Introgen Therapeutics, Inc.

(21) Appl. No.: 11/336,664

(22) Filed: Jan. 20, 2006

Related U.S. Application Data
(60) Provisional application No. 60/645,826, filed on Jan. 21, 2005.
(60) Provisional application No. 60/692,481, filed on Jun. 21, 2005.

(51) Int. Cl. A61K 48/00; A61K 9/68

(52) U.S. Cl. 514/44; 424/440; 424/48

(57) ABSTRACT

Compositions and methods for preventing or inhibiting the growth of a hyperproliferative lesion in a subject that include a nucleic acid comprised in a solid or semi-solid formation or in a transdermal or transcutaneous delivery device are disclosed. Also disclosed are compositions of a nucleic acid capable of preventing or inhibiting the growth of a hyperproliferative lesion in a subject that include an adhesive. Compositions of a nucleic acid capable of preventing or inhibiting the growth of a hyperproliferative lesion in a subject that include a nucleic acid uptake enhancer are also disclosed. Methods of preventing or inhibiting the growth of a hyperproliferative lesion in a subject that involve these therapeutic compositions and devices are also disclosed.

The present application is related to U.S. Provisional Patent Application 60/645,826, filed on Jan. 21, 2005, and U.S. Provisional Patent Application 60/692,481, filed on Jun. 21, 2005, both of which are hereby incorporated by reference in their entirety.

### BACKGROUND OF THE INVENTION

1. Field of the Invention

The present invention relates generally to the fields of gene transfer, gene therapy, pharmacology and pharmaceutics. More particularly, it concerns novel pharmaceutical compositions of nucleic acids that can be administered to detect, prevent or treat disease in a subject, and methods of detecting, preventing or treating disease using these pharmaceutical compositions. The pharmaceutical compositions are formulated as a liquid, semi-solid, or solid for topical application to a body surface of a subject, such as to a skin surface or a mucosal surface. The present invention also pertains to transcutaneous or transdermal delivery devices for delivery of diagnostic or therapeutic nucleic acids, and methods of diagnosing, preventing and treating disease in a subject using these devices.

2. Description of Related Art

Gene transfer is a relatively new modality that involves delivery of a particular gene particular target cells in a subject. Gene transfer for therapeutic purposes (i.e., gene therapy) involves the transfer of a therapeutic gene to target cells in a subject. Although originally envisioned as a treatment of single gene disorders, the majority of gene therapy trials pertain to the treatment of cancer and vascular disease.

There is great interest in the identification of gene therapy for cancer because cancer is the leading cause of death in the United States and elsewhere. A significant reason for the high morbidity and mortality associated with cancer is the fact that there are significant limitations in currently available diagnostic and therapeutic measures.

Many diagnostic measure are available, and examples include visual inspection (e.g., physical examination to identify skin lesions and colonoscopy to identify colon cancer), imaging studies such as mammography, CT and MRI, and blood tests (e.g., PSA as a marker for prostate cancer). Often, these measures fail to identify small foci of disease. In other instances, disease is far advances at the time of diagnosis.

Conventional therapies of cancer include surgery, chemotherapy, and/or radiation. These treatments are often unsuccessful: surgery may not remove all of the cancer; some cancers are resistant to chemotherapy and radiation therapy; and chemotherapy-resistant tumors frequently develop.

Gene therapy has shown promise in the treatment of cancer. The goal of gene therapy in cancer therapy is the reestablishment of normal control of cellular proliferation or the elimination of cells undergoing aberrant proliferation. There are various strategies by which in vivo genetic modification can lead to therapeutic benefit. Exemplary strategies include the enhancement of immunogenicity toward the aberrant cells, the correction of a genetic defect which leads to the aberrant phenotype and the delivery of a gene whose product is or can be made toxic to the recipient cells.

An exemplary category of therapeutic genes that can be considered for gene therapy of cancer includes tumor suppressor genes. Tumor suppressor genes are genes that normally restrain cell growth but, when missing or inactivated by mutation, allow cells to grow uncontrolled. One of the best known tumor suppressor genes is p53, which plays a central role in cell cycle progression, arresting growth so that repair or apoptosis can occur in response to DNA damage. It can also initiate apoptosis if the DNA damage proves to be irreparable.

Regardless of which gene is used to reinstate the control of cell cycle progression, the rationale and practical applicability of this approach is identical. Namely, to achieve high efficiencies of gene transfer to express therapeutic quantities of the recombinant product.

One aspect of successful gene therapy of cancer or other diseases is the ability to affect a significant fraction of the aberrant cells. Viral vectors are employed for this purpose. Recombinant adenoviruses have distinct advantages over retroviral and other gene delivery methods (reviewed in Siegfried, 1993). Adenoviruses have never been shown to induce tumors in humans and have been safely used as live vaccines (see Straus, 1984). Replication deficient recombinant adenoviruses can be produced by replacing the E1 region necessary for replication with the target gene. Adenovirus does not integrate into the human genome as a normal consequence of infection, thereby greatly reducing the risk of insertional mutagenesis. Stable, high titer recombinant adenovirus can be produced, allowing enough material to be produced to treat a large patient population. Moreover, adenovirus vectors are capable of highly efficient in vivo gene transfer into a broad range of tissue and tumor cell types.

Although viral vectors offer several advantages over other modes of gene delivery vehicles, they still exhibit some characteristics which impose limitations to their efficient use in vivo. These limitations primarily result in the limited ability of the vectors to efficiently deliver and target therapeutic genes to the aberrant cells. Attempts have been made to overcome this problem by direct injection of large quantities of viral vectors into the region containing the target cells. Current local administration of virus vectors is by injection of approximately 1×10¹² viral particles into the region of the target cells. Unfortunately, a high proportion of this material is not retained in the area of injection, but is quickly cleared through the circulatory and lymphatic systems, thus preventing infection of the target cells.

Besides virus-mediated gene-delivery systems, there are several nonviral options for gene delivery. One nonviral approach involves the use of liposomes to carry the therapeutic gene. Another approach, which is limited in application, is the direct introduction of therapeutic DNA into target cells.

Besides gene transfer as a form of therapy, a few studies have described applications of gene transfer in imaging. A new form of imaging that has developed during the past decade involves the in situ or in vivo imaging of a reporter gene. Reporter gene technology was first applied to in situ imaging of tissue sections (reviewed in Blasberg et al., 2003). For example, Hooper et al. (1990) described imaging of luciferase gene expression in single mammalian cells. Reporter imaging has been described as being based on magnetic resonance, nuclear imaging (PET, gamma camera) and/or in vivo optical imaging systems (reviewed in Blasberg et al., 2003). For example, transfer of the herpes simplex virus-1 thymidine kinase or dopamine receptor type-2 has been detected by positron emission tomography (PET) (Alauddin et al., 1996; Alauddin and Conti, 1998; Gambhir et al., 1998; MacLaren et al., 1999; Tjuvajev et al., 1998). In comparison, transfer of the sodium-iodide symporter (Mandell, 1999), dopamine transporter (Auricchio et al., 2003), or the somatostatin receptor type-2 (Kundra, 2002; Sun et al., 2001) has been detected by gamma camera imaging. It remains to be determined whether any of these measures can be applied in diagnosing human disease.

Thus, there exists a need for new and improved compositions and methods of gene transfer in the diagnosis and treatment of disease, such as cancer. For example, compositions of therapeutic nucleic acids which allow for prolonged contact of the nucleic acid with the appropriate target cells would improve therapeutic efficacy of the formulation. Methods of delivery of a reporter gene to diseased cells of a subject might provide for more improved ability to target and detect diseased cells.

### SUMMARY OF THE INVENTION

The inventors have identified certain novel formulations of nucleic acids and methods of applying these formulations in the diagnosis, treatment, and prevention of disease. The nucleic acids of the formulations set forth herein can be any nucleic acid that can be of use in the diagnosis, prevention, or treatment of a disease. For example, the nucleic acid may be a nucleic acid encoding an amino acid sequence that is capable of promoting wound healing or treating the growth of a hyperproliferative lesion in a subject.

These novel formulations of nucleic acids facilitate more efficient delivery and targeting of a nucleic acid of interest to target cells in a subject. For example, some of the compositions are formulated with an adhesive to result in prolonged contact of therapeutic nucleic acid with the target cells of interest.

The inventors have also discovered novel transdermal or transcutaneous delivery devices for delivery of diagnostic or therapeutic nucleic acid sequences. For example, the device may be designed to deliver a nucleic acid that encodes a protein capable of inhibiting the growth of a hyperproliferative lesion in a subject.

Methods of applying these novel formulations and devices in the diagnosis, prevention or treatment of diseases amenable to gene therapy have also been identified.

More specifically, certain embodiments of the present invention generally pertain to pharmaceutical compositions that include a therapeutic nucleic acid and/or a diagnostic nucleic acid that is formulated for application to a surface of a subject. The subject can be any subject, such as a mammal or avian species. In particular embodiments, the subject is a human, such as a human with cancer.

The surface of the subject can be any surface. The term “surface” is used according to its ordinary and plain meaning in the context of a biological organism, meaning “the outside of an animal body, or of any part of it; the outer boundary of the integument; also, the inner boundary of a hollow or tubular part.” For example, the surface may be a skin surface, a mucosal surface, the surface of a lesion, the surface of the wound, or the surface of a hollow viscus. The skin surface may be normal skin, or it may be the surface of a skin lesion, such as a skin cancer (e.g., basal cell carcinoma, squamous cell carcinoma). A mucosal surface may be any mucosal surface of the body, such as the surface of the oral cavity, the surface of the esophagus, lung mucosal surface, stomach, duodenum, small intestine, large intestine, colon, rectum, vagina, or bladder. The mucosal surface may be normal mucosa, or it may be the surface of a lesion of the mucosa, such as a leukoplakia of the mouth, colon polyp, or tumor. The surface of a lesion may be any lesion, whether benign, premalignant, or malignant. The surface may be a wound surface, such as a traumatic wound or a post-surgical wound such as a wound following surgical resection of a tumor. The surface may be a surface of an internal organ, such as the surface of the gastrointestinal tract, surface of the bladder, vagina, cervix, or the uterus. The surface may be pretreated, such as abraded, as discussed in detail below, to allow for more efficient transfer to underlying tissue. Formulation for application to a surface does not imply that the formulation might not later be found suitable for application by other means, such as intravenous administration. Furthermore, it is contemplated that certain of the nucleic acid formulations set forth herein may be suitable for formulation to one surface, such as a wound surface, and not suitable for application to other surfaces, such as the surface of the stomach.

Any type of nucleic acid is contemplated for inclusion in compositions and devices set forth herein, and includes, for example, DNA, RNA of all types, such as siRNA, RNAi, microRNA, ribozymes, and CpG oligonucleotides.

A “therapeutic nucleic acid” is defined herein to refer to a nucleic acid that is known or suspected to be of benefit in the treatment or prevention of a disease or health-related condition. For example, the “therapeutic nucleic acid” may be a nucleic acid that encodes a protein or polypeptide that is known or suspected to be of benefit in the treatment of a disease or health-related condition. Also included in the definition of “therapeutic nucleic acid” is a nucleic acid that transcribes a second nucleic acid that is known or suspected to be of benefit in the treatment of a disease or health-related condition (e.g., a DNA transcribed into ribozyme or siRNA). Alternatively, the “therapeutic nucleic acid” may be one which is known or suspected to provide for a therapeutic benefit without undergoing transcription (e.g., a siRNA or a ribozyme).

Therapeutic benefit may arise, for example, as a result of alteration of expression of a particular gene or genes by the nucleic acid. Alteration of expression of a particular gene or genes may be inhibition or augmentation of expression of a particular gene. In particular embodiments of the present invention, the therapeutic nucleic acid encodes one or more proteins or polypeptides that can be applied in the treatment or prevention of a disease or health-related condition in a subject.

A “disease” is defined as a pathological condition of a body part, an organ, or a system resulting from any cause, such as infection, genetic defect, or environmental stress. A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like. The disease can be any disease, and non-limiting examples include hyperproliferative diseases such as cancer and premalignant lesions, wounds, and infections.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

The therapeutic nucleic acid may encode a therapeutic protein, such as a tumor suppressor, a proapoptotic protein (meaning a protein that promotes apoptosis), a cytokine, a growth factor, a hormone, a tumor antigen, or an enzyme. Examples of tumor suppressor genes include mda7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 10F6, Gene 21 (NPRL2), or a gene encoding a SEM A3 polypeptide. In particular embodiments, the tumor suppressor is p53 and/or FUS1. Examples of pro-apoptotic genes include CD95, caspase-3, Bax, Bag-1, CRADD, TSSC3, bax, hid, Bak, MKP-7, PARP, bad, bcl-2, MST1, bbc3, Sax, BIK, and BID. Examples of cytokines include GM-CSF, G-CSF, IL-1α, IL-1β, IL-2, IL-3, IL-4, IL-5, IL-6, IL-7, IL-8, IL-9, IL-10, IL-1, IL-12, IL-13, IL-14, IL-15, IL-16, IL-17, IL-18, IL-19, IL-20, IL-21, IL-22, IL-23, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, PDGF, TGF-α, TGF-β, VEGF and mda7. In particular embodiments, the cytokine is mda7.

The nucleic acid may encode a tumor antigen. The tumor antigen may be any tumor antigen known to those of ordinary skill in the art. Examples of tumor antigens include: MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-3, MAGE-4, MAGE-5, MAGE-6, and other members of the MAGE gene family, p185erbB2, p180erbB-3, c-met, mn-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, ING1, mamaglobin, cyclin B1, S100, BRCA1, BRCA2, a tumor immunoglobulin idiotype, a tumor T-cell receptor clonotype, MUC-1, or epidermal growth factor receptor, a tumor suppressor, or a peptide of any of the aforementioned tumor-associated antigens. oncogenes, or a pep. The nucleic acid may comprise a tumor suppressor gene, or a wild-type or mutated form of an oncogene or tumor suppressor gene. Examples of tumor antigens include antigens formed by chromosome translocations or oncogene/tumor suppressor gene mutations (e.g., bcr/abl, ras); developmental/differentiation antigens (e.g. MUC-1, MAGE, tyrosinase, melan-A and gp75); antigens up regulated in malignant transformation (oncofetal antigens—carcinoembryonic antigen/CEA, alphafetoprotein/AFP, growth factor receptors-Her2/neu, telomerase, and p53) and viral antigens associated with tumor pathogenesis (hepatitis, papilloma and Epstein-Barr viruses) and di(MUC-1, Melan-A).

Examples of growth factors include epidermal growth factor, keratinocyte growth factor, and hepatocyte growth factor. Examples of additional therapeutic proteins, including hormones and enzymes, are discuss in the specification below. It is specifically contemplated that any of the proteins identified in this paragraph may be considered part of the invention; in addition, it is specifically contemplated that one or more of these proteins is also not considered part of the invention in some embodiments.

A “diagnostic nucleic acid” is a nucleic acid that is known or suspected to be of benefit in identifying the presence or absence of a disease or health-related condition, or that is known or suspected to be of benefit in identifying a subject at risk of developing a particular disease or health-related condition. Also included in the definition of “diagnostic nucleic acid” is a nucleic acid sequence that encodes one or more reporter proteins. A “reporter protein” refers to an amino acid sequence that, when present in a cell or tissue, is detectable and distinguishable from other genetic sequences or encoded polypeptides present in cells. A reporter protein may be a naturally occurring protein or a protein that is not naturally occurring. If naturally occurring, it may be detectable as a result of the amount of expression following gene transfer, or it may be a protein to which a detectable tag can be attached. Examples of such reporter proteins include fluorescent proteins such as green fluorescent protein (gfp), cyan fluorescent protein (cfp), red fluorescent protein (rfp), or blue fluorescent protein (bfp), or derivatives of these proteins, or enzymatic proteins such as β-galactosidase, chemiluminesent proteins such as luciferase, somatostatin receptor amino acid sequence, a sodium iodide symporter amino acid sequence, a luciferase amino acid sequence, and a thymidine kinase amino acid sequence. These and other reporter proteins are discussed in greater detail in the specification below.

Some of the novel pharmaceutical compositions set forth herein pertain to compositions of a therapeutic nucleic acid and/or a diagnostic nucleic acid wherein the formula is an aqueous formulation. Examples of aqueous formulations include mouthwashes, mouthrinses, douches, enemas, sprays, and aerosols.

Additional formulations include a dispersion, an emulsion, a microemulsion, a suspension, a matrix, a microparticle, a microcapsule, an emulsion, a microemulsion, or a dispersion.

Other compositions are formulated as a solid or semi-solid. Solid and semi-solid formulations refer to any formulation other than aqueous formulations. In specific embodiments, it is contemplated that a solid or semi-solid is not a pill or tablet, such as for oral administration. Examples include a gel, a matrix, a foam, a cream, an ointment, a lozenge, a lollipop, a popsicle a gum, a powder, a gel strip, a film, a hydrogel, a dissolving strip, a paste, a toothpaste, or a solid stick. In certain embodiments, the invention does not specifically include one or more of a lozenge, a lollipop, a popsicle, a gum, a gel strip, a film a hydrogel, a dissolving strip, or a solid stick.

Regarding solid or semi-solid formulations, any formulation of the pharmaceutical compositions of the present invention that is a solid or semi-solid is contemplated for inclusion in the present invention. These are addressed at length elsewhere in this specification. The formulation may include any number of additional excipients, as discussed in greater detail below. Examples include collagen, glycerin, PEG, hydrated silica, cellulose, xanthum gum, glycan carbomer 956, Tween 80, fluoride, carrageenan, an adhesive and/or a nucleic acid uptake enhancer. In some embodiments, the excipients may also include cosmetic ingredients, as discussed in greater detail below.

As discussed in greater detail below, the pharmaceutical compositions set forth herein may include any number of additional therapeutic and/or diagnostic agents. Examples include additional therapeutic agents, an antacid, and alginate-raft forming components.

In certain particular embodiments, the pharmaceutical composition includes a therapeutic and/or a diagnostic nucleic acid, wherein the composition is formulated as a lozenge, a lollipop, a popsicle, a gum, a gel strip, a film, a hydrogel, a dissolving strip, a cream, a salve, a suppository, or a solid stick.

The pharmaceutical compositions of therapeutic and/or diagnostic nucleic acids set forth herein may further include one or more adhesive. An “adhesive” is defined herein to generally refer to an agent or combination of agents that promotes or facilitates contact of the nucleic acid with a surface, or promotes or facilitates contact of one surface with another surface. Any adhesive known to those of ordinary skill in the art that is suitable for pharmaceutical purposes is contemplated as an adhesive that can be included in the pharmaceutical compositions and devices of the present invention. For example, the adhesive may be an acrylate, a hydrocolloid, a hydrogel, a polyacrylic acid-based gel matrix, a polyisobutylene, a silicone polymer, or a mixture thereof. Adhesives are discussed in detail in the specification below. Exemplary types of acrylate adhesives include cyanoacrylates, methacrylates, or alkyl acrylates.

Any nucleic acid uptake enhancer known to those of ordinary skill in the art is contemplated for inclusion in the present pharmaceutical compositions set forth herein. A “nucleic acid uptake enhancer” is defined herein to refer to any agent or composition of more than one agents that can be applied to the surface of a cell or contacted with the surface of a cell to facilitate uptake of a nucleic acid that is external to the cell. Exemplary cationic lipids include quaternary cytofectin, bis-guanidinium-tren-cholesterol, and 1,2-dioleoyl-3-(trimethyammonium)propate (DOTAP). These agents are addressed in greater detail in the specification below.

In some embodiments, the solid or semi-solid pharmaceutical composition is formulated as a cosmetic. The cosmetic may be in the form of a lipstick, salve, cream, paste, gel or lotion. Additional excipients, such as colorants, may also be included, such as, waxes, oils, humectants, preservatives, antioxidants, ultraviolet absorbers, ultraviolet scattering agents, polymers, surface active agents, colorants, pigments, powders, drugs, alcohols, solvents, fragrances, or flavors.

In pharmaceutical composition may be formulated as a toothpaste, and may include one or more additional agents that are commonly present in toothpastes, such as fluoride, flavorants, and whitening agents.

In some other embodiments, the pharmaceutical composition is formulated as a gum. The gum may be a chewing gum. Additional excipients, such as sweeteners and flavorants, may be included in the formulation. The gum, in some embodiments, includes xanthum gum.

In some embodiments of the present invention, the pharmaceutical composition has been lyophilized. One of ordinary skill in the art would be familiar with lyophilization.

The nucleic acid may be comprised in an expression cassette that includes a promoter operatively coupled to the nucleic acid, wherein the promoter is active in cells of the subject. The expression cassette may be carried in a viral vector. One of ordinary skill in the art would be familiar with the many types of viral vectors that are available. For example, the viral vector may be an adenoviral vector, a baculovirus vector, a parvovirus vector, a semiliki forest virus vector, an alpha virus vector, a parvovirus vector, a Sindbis virus vector, a lentivirus vector, a retroviral vector, a vaccinia viral vector, an adeno-associated viral vector, or a poxviral vector. In certain particular embodiments, the viral vector is an adenoviral vector, such as an adenoviral vector that includes a nucleic acid encoding p53, mda7, or FUS1. In some embodiments, the viral vector is an oncolytic virus. Oncolytic viruses are discussed in detail in the specification below. Examples of oncolytic viruses include viruses that overexpress ADP, and viruses such as Ad5, dl327, pm734.1, dl309, dl01/07, KD1, KD2, KD3, dl1520 and VRX-007. The pharmaceutical composition that includes a viral vector may or may not be lyophilized.

In further embodiments, the pharmaceutical composition that includes a therapeutic and/or diagnostic nucleic acid includes one or more delivery agents. A “delivery agent” is defined herein to refer to any agent or substance, other than a viral vector, that facilitates the delivery of the nucleic acid to a target cell of interest. One of ordinary skill in the art would be familiar with the various types of delivery agents that are available. For example, the delivery agent may be a lipid. The lipid may or may not be comprised in a liposome. Liposomal formulations are well-known in the art. In some embodiments, DOTAP:cholesterol nanoparticles are the delivery agent.

The expression cassettes of the compositions and devices of the present invention may include any type of promoter, as long as the promoter is active in a cell of the subject. For example, the promoter may a constitutive promoter, an inducible promoter, a repressible promoter, or a tissue selective promoter. A tissue selective promoter is defined herein to refer to any promoter which is relatively more active in certain tissue types compared to other tissue types. Thus, for example, a liver-specific promoter would be a promoter which is more active in liver compared to other tissues in the body. One type of tissue-selective promoter is a tumor selective promoter. A tumor selective promoter is defined herein to refer to a promoter which is more active in tumor tissue compared to other tissue types. There may be some function in other tissue types, but the promoter is relatively more active in tumor tissue compared to other tissue types. Examples of tumor selective promoters include the hTERT promoter, the CEA promoter, the PSA promoter, the probasin promoter, the ARR2PB promoter, and the AFP promoter.

In some embodiments of the present invention, the pharmaceutical composition is a non-adenoviral composition that includes a therapeutic nucleic acid and/or a diagnostic nucleic acid, wherein the composition is formulated as a gel, a paste, a foam, a slurry, a cream, a salve, a suppository, or a powder. In particular aspects, the composition comprises a nucleic acid encoding p53, mda7, and/or FUS1.

The pharmaceutical composition may be formulated to be administered via a transdermal patch, a strip, a bandage, a tape, a dressing, or synthetic skin. These formulations are discussed in greater detail below.

The present invention also generally pertains to transdermal or transcutaneous delivery devices for delivery of a therapeutic or diagnostic agent to a subject, that include a patch and a pharmaceutical composition that includes a nucleic acid encoding a reporter protein, a tumor suppressor, a pro-apoptotic protein, a growth factor, or a cytokine, wherein the pharmaceutical composition is applied to at least one surface of the patch. The discussion above pertaining to pharmaceutical compositions applies herein to these transdermal or transcutaneous delivery devices. Exemplary tumor suppressors, pro-apoptotic proteins, growth factors, reporters, and cytokines are discussed elsewhere in this specification. As set forth above, the nucleic acid may be comprised in an expression cassette that comprises a promoter operatively coupled to the nucleic acid, wherein the promoter is active in cells in the subject. The discussion above pertaining to expression cassettes applies herein to this section. In particular embodiments, the expression cassette is a viral vector, such as an adenoviral vector. In some embodiments, the nucleic acid is a therapeutic nucleic acid encoding p53, mda7, or FUS1.

Embodiments of the present invention also pertain to methods of detecting, preventing or treating disease in a subject that involves administering to the subject any of the pharmaceutical compositions set forth above. Further, embodiments of the present invention also pertain to methods of detecting, preventing, or treating disease in a subject that involves applying to a body surface of the subject one or more of the transdermal or transcutaneous delivery devices set forth herein.

In some examples, the nucleic acid may encode a reporter protein, and wherein the method is further defined as a method of detecting a lesion in a subject.

The disease may be any disease. For example, the disease may be a hyperproliferative lesion. Exemplary hyperproliferative lesions include pre-malignant lesions, cancer, and tumors. The hyperproliferative lesion, pre-malignant lesion or cancer may be breast cancer, lung cancer, prostate cancer, ovarian cancer, brain cancer, liver cancer, cervical cancer, cervical dysplasia, colon cancer, renal cancer, skin cancer, dysplastic nevi, head and neck cancer, bone cancer, esophageal cancer, hyperkeratosis, kyphosis, seborrheic keratosis, bladder cancer, uterine cancer, lymphatic cancer, stomach cancer, pancreatic cancer, testicular cancer, lymphoma, leukemia or dysplastic lesions of these same tissues or organs. Other diseases include diabetic ulcers, venous stasis ulcers, decubitus ulcers, burns, wounds, and mucositis

In certain embodiments, the hyperproliferative lesion is a disease that can affect the mouth of a subject. Examples include leukoplakia, squamous cell hyperplastic lesions, premalignant epithelial lesions, oral dysplasia, intraepithelial neoplastic lesions, focal epithelial hyperplasia, and squamous carcinoma lesion.

The subject can be any subject, such as a mammal. In certain embodiments, the mammal is a human. For example, the human may be a patient with a premalignant lesion or a patient with cancer. In certain embodiments, the subject is undergoing secondary treatment for a hyperproliferative lesion, such as secondary anti-cancer therapy. Examples of such therapy, which are discussed in greater detail in the specification below, include surgical therapy, chemotherapy, radiation therapy, and immunotherapy.

The nucleic acid may be a therapeutic nucleic acid, such as a nucleic acid that encodes a tumor suppressor, a proapoptotic protein, a cytokine, or a growth factor. These are discussed in greater detail above and elsewhere in this specification. The nucleic acid may further be a diagnostic nucleic acid, such as a nucleic acid encoding a reporter protein as discussed above. In other embodiments, it is specifically contemplated that the therapeutic nucleic acid specifically does not encode a tumor suppressor, a proapoptotic protein, a cytokine, or a growth factor, or any of the specific such proteins discussed herein.

In some embodiments, the method is further defined as a method of promoting healing of a wound of the subject. In these embodiments, for example, the nucleic acid may encode a growth factor, such as those discussed above. In further embodiments, the nucleic acid is a therapeutic nucleic acid, and the method is further defined as a method of preventing or inhibiting the growth of a hyperproliferative lesion in a subject. For example, the hyperproliferative lesion may be oral dysplasia or leukoplakia in the subject. The method may further include identification of a subject in need of detection, treatment, or prevention of a disease or health-related condition. Examples of ways of identifying a subject at risk include clinical screening based on history or examination, interview by a physician, or completion of a questionnaire to identify such risk factors.

As set forth above, the nucleic acid may be comprised in an expression cassette comprising a promoter operatively coupled to the nucleic acid, wherein the promoter is active in cells of the subject. In particular embodiments, the expression cassette is carried in a viral vector such as an adenoviral vector. In more particular embodiments, the expression cassette is carried in an adenoviral vector, and the nucleic acid encodes p53, mda7, or FUS1.

Any method of administering the pharmaceutical composition known to those of ordinary skill in the art is contemplated by the present methods. “Administering” includes providing the pharmaceutical composition to the subject. One of ordinary skill in the art would be familiar with the many ways by which a pharmaceutical composition could be administered. For example, administration may involve topically applying a formulation to a body surface of the subject. For example, an applicator may be used for application of a gel or paste, such as using a cotton-tipped applicator and spatula. The applicator may or may not be disposable. The composition may be applied by any individual, such as a health care professional or the subject to whom the composition is administered. Also contemplated in the definition of “administering” is prescribing the pharmaceutical composition, such as prescription by a health care professional. The pharmaceutical compositions set forth herein may be in the form of a kit that includes a disposable or reusable applicator and the pharmaceutical composition. Such a kit may be designed for application of the pharmaceutical composition by a health care provider or the subject.

The therapeutic methods set forth herein may include administration of one or more secondary forms of therapy to the subject. Secondary forms of therapy include any known to those of ordinary skill in the art, and are largely dependent on the disease process. Examples are set forth in the specification below.

Certain of the nucleic acids set forth herein may not be amenable to each and every formulation set forth herein. Thus, for example, a particular nucleic acid suitable for formulation as a cream may not necessarily be suitable for formulation as a lozenge.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Scheme for generation of recombinant p53 adenovirus. The p53 expression cassette was inserted between the Xba I and Cla I sites of pXCJL.1. The p53 expression vector (pEC53) and the recombinant plasmid pJM17 were cotransfected into 293 cells. The transfected cells were maintained in medium until the onset of the cytopathic effect. Identification of newly generated p53 recombinant adenoviruses (AdCMV-p53) by PCR analysis of the DNA using DNA templates prepared from the CPE supernatants treated with Proteinase K and phenol extraction.

### DESCRIPTION OF ILLUSTRATIVE EMBODIMENTS

The inventors have identified certain novel compositions of nucleic acids that can be used in the diagnosis, treatment, and/or prevention of disease in a subject. These compositions include a nucleic acid that is formulated, for example, for application to a body surface of a subject, such as the skin, the surface of a lesion, a mucosal surface, a wound surface, a tumor surface, or the lining of a hollow viscus, such as the stomach. In some embodiments the nucleic acid encodes a reporter gene that can be applied in the diagnosis of a disease. Also set forth are novel methods of diagnosing and treating disease in a subject that involve use of the novel formulations of nucleic acids set forth herein. The novel compositions and methods set forth herein can be applied in the detection, prevention or treatment of any of a number of diseases and health-related conditions. Examples of such diseases include cancer, and infection, and wound healing. Applications of these novel compositions in the diagnosis, treatment, and prevention of disease represents an improvement in existing gene therapy technology.

### A. NUCLEIC ACIDS

1. Nucleic Acids in General

The pharmaceutical compositions and methods of the present invention involve nucleic acids that are known or suspected to be of benefit in the diagnosis, treatment, or prevention of a disease or health-related condition in a subject.

The term “nucleic acid” is well known in the art. A “nucleic acid” as used herein will generally refer to a molecule (i.e., a strand) of DNA, RNA (including RNAi siRNA, and ribozymes), and oligonucleotide, an oligonucleotide comprising CpG site, or a derivative or analog thereof, comprising a nucleobase. The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand. The additional strand may be partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule As used herein, a single stranded nucleic acid may be denoted by the prefix “ss,” a double stranded nucleic acid by the prefix “ds,” and a triple stranded nucleic acid by the prefix “ts.”

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, carboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Table 1 shows non-limiting examples of purine and pyrimidine derivatives and analogs.

A nucleobase may be comprised in a nucleoside or nucleotide, using any chemical or natural synthesis method described herein or known to one of ordinary skill in the art.

TABLE 1
Purine and Pyrmidine Derivatives or Analogs

| Abbr. | Modified base description |
|---|---|
| ac4c | 4-acetylcytidine |
| Chm5u | 5-(carboxyhydroxylmethyl) |
| | uridine |
| Cm | 2′-O-methylcytidine |
| Cmnm5s2u | 5-carboxymethylamino- |
| | methyl-2-thioridine |
| Cmnm5u | 5-carboxymethylaminomethyluridine |
| D | Dihydrouridine |
| Fm | 2′-O-methylpseudouridine |
| Gal q | Beta, D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta, D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2- |
| | methylthiopurine-6-yl)- |
| | carbamoyl)threonine |
| Mt6a | N-((9-beta-D-ribofuranosylpurine-6-yl)- |
| | N-methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

c. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

d. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference). Any derivative or analog of a nucleoside or nucleotide that is known to those of ordinary skill in the art may be used in the methods and compositions of the present invention. A non-limiting example is a “polyether nucleic acid” and a “peptide nucleic acid.”

e. Preparation of Nucleic Acids

A nucleic acid may be made by any technique known to one of ordinary skill in the art. Examples include chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques. A non-limiting example of an enzymatically produced nucleic acid includes one produced by enzymes in amplification reactions such as PCR™ and other techniques known to those of ordinary skill in the art (see, e.g., U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

f. Nucleic Acid Complements

The present invention also encompasses a nucleic acid that is complementary to a nucleic acid encoding an amino acid sequence capable of diagnosing, treating, or preventing disease in a subject. A nucleic acid “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70% to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

2. Therapeutic Nucleic Acids

In some embodiments of the formulations set forth herein, the nucleic acid is a therapeutic nucleic acid. A “therapeutic nucleic acid” is defined herein to refer to a nucleic acid which can be administered to a subject for the purpose of treating or preventing a disease. The nucleic acid is one which is known or suspected to be of benefit in the treatment of a disease or health-related condition in a subject. Diseases and health-related conditions are discussed at length elsewherein this specification.

Therapeutic benefit may arise, for example, as a result of alteration of expression of a particular gene or genes by the nucleic acid. Alteration of expression of a particular gene or genes may be inhibition or augmentation of expression of a particular gene. In certain embodiments of the present invention, the therapeutic nucleic acid encodes one or more proteins or polypeptides that can be applied in the treatment or prevention of a disease or health-related condition in a subject. The terms “protein” and “polypeptide” are used interchangeably herein. Both terms refer to an amino acid sequence comprising two or more amino acid residues.

Any nucleic acid known to those of ordinary skill in the art that is known or suspected to be of benefit in the treatment or prevention of a disease or health-related condition is contemplated by the present invention as a therapeutic nucleic acid. The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein. In some embodiments, the nucleic acid includes a therapeutic gene. The term “gene” is used to refer to a nucleic acid sequence that encodes a functional protein, polypeptide, or peptide-encoding unit.

As will be understood by those in the art, the term “therapeutic nucleic acid” includes genomic sequences, cDNA sequences, and smaller engineered gene segments that express, or may be adapted to express, proteins, polypeptides, domains, peptides, fusion proteins, and mutants. The nucleic acid may comprise a contiguous nucleic acid sequence of about 5 to about 12000 or more nucleotides, nucleosides, or base pairs.

Encompassed within the definition of “therapeutic nucleic acid” is a “biologically functional equivalent” of a therapeutic nucleic acid that has proved to be of benefit in the treatment or prevention of a disease or health-related condition. Accordingly, sequences that have about 70% to about 99% homology to a known nucleic acid are contemplated by the present invention.

a. Nucleic Acids that Encode Tumor Suppressors and Pro-Apoptotic Proteins

In some embodiments, the nucleic acid of the claimed pharmaceutical compositions include a nucleic acid sequence that encodes a protein or polypeptide that can be applied in the treatment or prevention of cancer or other hyperproliferative disease. Examples of such proteins include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, IL-13, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

A “tumor suppressor” refers to a polypeptide that, when present in a cell, reduces the tumorigenicity, malignancy, or hyperproliferative phenotype of the cell. The nucleic acid sequences encoding tumor suppressor gene amino acid sequences include both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

A nucleic acid encoding a tumor suppressor generally refers to a nucleic acid sequence that reduce the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressors include, but are not limited to, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes at www.cise.ufl.edu/˜yy1/HTML-TSGDB/Homepage.html. This database is herein specifically incorporated by reference into this and all other sections of the present application. Nucleic acids encoding tumor suppressor genes, as discussed above, include tumor suppressor genes, or nucleic acids derived therefrom (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

One of the best known tumor suppressor genes is p53. p53 is central to many of the cell's anti-cancer mechanisms. It can induce growth arrest, apoptosis and cell senescence. In normal cells p53 is usually inactive, bound to the protein MDM-2, which prevents its action and promotes its degradation. Active p53 is induced after the effects of various cancer-causing agents such as UV radiation, oncogenes and some DNA-damaging drugs. DNA damage is sensed by ‘checkpoints’ in a cell's cycle, and causes proteins such as ATM, Chk1 and Chk2 to phosphorylate p53 at sites that are close to the MDM2-binding region of the protein. Oncogenes also stimulate p53 activation, mediated by the protein p14ARF. Some oncogenes can also stimulate the transcription of proteins which bind to MDM2 and inhibit its activity. Once activated p53 has many anticancer mechanisms, the best documented being its ability to bind to regions of DNA and activate the transcription of genes important in cell cycle inhibition, apoptosis, genetic stability, and inhibition of angiogenesis (Vogelstein et al, 2000). Studies have linked the p53 and pRB tumour suppressor pathways, via the protein p14ARF, raising the possibility that the pathways may regulate each other (Bates et al, 1998).

A nucleic acid encoding a pro-apoptotic protein encode a protein that induces or sustains apoptosis to an active form. The present invention contemplates inclusion of any nucleic acid encoding a pro-apoptotic protein known to those of ordinary skill in the art. Exemplary pro-apoptotic proteins include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic proteins, including those not specifically set forth herein.

Nucleic acids encoding pro-apoptotic amino acid sequences include, for example, cDNAs, cRNAs, mRNAs, and subsequences thereof encoding active fragments of the respective pro-apoptotic amino acid sequence.

One of ordinary skill in the art would understand that there are other nucleic acids encoding proteins or polypeptides that can be applied in the treatment of a disease or health-related condition that are not specifically set forth herein. Further, it is to be understood that any of the therapeutic nucleic acids mentioned elsewhere in this specification, such as nucleic acids encoding cytokines, may be applied in the treatment and prevention of cancer.

b. Nucleic Acids Encoding Cytokines

In some embodiments of the pharmaceutical compositions set forth herein the nucleic acid encodes a cytokine. The term “cytokine” is a generic term for proteins released by one cell population which act on another cell as intercellular mediators. The nucleic acid sequences may encode the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factors (FGFs) such as FGF-α and FGF-β; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-α; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-11, IL-12; IL-13, IL-14, IL-15, IL-16, IL-17, IL-18, LIF, G-CSF, GM-CSF, M-CSF, EPO, kit-ligand or FLT-3.

A non limiting example of growth factor cytokines involved in wound healing include: epidermal growth factor, platelet-derived growth factor, keratinocyte growth factor, hepatycyte growth factor, transforming growth factors (TGFs) such as TGF-α and TGF-β, and vascular endothelial growth factor (VEGF). These growth factors trigger mitogenic, motogenic and survival pathways utilizing Ras, MAPK, PI-3K/Akt, PLC-gamma and Rho/Rac/actin signaling. Hypoxia activates pro-angiogenic genes (e.g., VEGF, angiopoietins) via HIF, while serum response factor (SRF) is critical for VEGF-induced angiogenesis, re-epithelialization and muscle restoration. EGF, its receptor, HGF and Cox2 are important for epithelial cell proliferation, migration re-epithelializaton and reconstruction of gastric glands. VEGF, angiopoietins, nitric oxide, endothelin and metalloproteinases are important for angiogenesis, vascular remodeling and mucosal regeneration within ulcers. (Tarnawski, 2005)

Another example of a cytokine is IL-10. IL-10 is a pleiotropic homodimeric cytokine produced by immune system cells, as well as some tumor cells (Ekmekcioglu et al., 1999). Its immunosuppressive function includes potent inhibition of proinflammatory cytokine synthesis, including that of IFNγ, TNFα, and IL-6 (De Waal et al., 1991). The family of IL-10-like cytokines is encoded in a small 195 kb gene cluster on chromosome 1q32, and consists of a number of cellular proteins (IL-10, IL-19, IL-20, MDA-7) with structural and sequence homology to IL-10 (Kotenko et al., 2000; Gallagher et al., 2000; Blumberg et al., 2001; Dumoutier et al., 2000; Knapp et al., 2000; Jiang et al., 1995a; Jiang et al., 1996).

A recently discovered putative member of the cytokine family is MDA-7. MDA-7 has been characterized as an IL-10 family member and is also known as IL-24. Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3′UTR targeting mRNA for rapid degradation, MDA-7 has three AREs in its 3′UTR¹⁷. Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405).

c. Nucleic Acids Encoding Enzymes

Other examples of therapeutic nucleic acids include nucleic acids encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. However, in certain embodiments of the invention, it is contemplated that the invention specifically does not include one or more of the enzymes identified above or in the following paragraph.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, glucosyltransferase, HSV thymidine kinase, or human thymidine kinase.

A therapeutic nucleic acid of the present invention may encode a superoxide dismutase (SOD). SOD, which exists in several isoforms, is a metalloenzyme which detoxifies superoxide radicals to hydrogen peroxide. Two isoforms are intracellular: Cu/Zn-SOD, which is expressed in the cytoplasm, and Mn-SOD, which is expressed in mitochondria (Linchey and Fridovich, 1997). Mn-SOD has been demonstrated to increase resistance to radiation in hematopoetic tumor cell lines transfected with MnSOD cDNA (Suresh et al., 1993). Adenoviral delivery of Cu/Zn-SOD has been demonstrated to protect against ethanol induced liver injury (Wheeler et al., 2001). Additionally adenoviral mediated gene delivery of both Mn-SOD and Cu/Zn-SOD are equally efficient in protection against oxidative stress in a model of warm ischemia-reprofusion (Wheeler et al., 2001).

d. Nucleic Acids Encoding Hormones

Therapeutic nucleic acids also include nucleic acids encoding hormones. Examples include, but are not limited to, growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, and thyrotropin releasing hormone.

Other examples of therapeutic genes include genes encoding antigens present in pathogens, or immune effectors involved in autoimmunity. These genes can be applied, for example, in formulations that would be applied in vaccinations for immune therapy or immune prophylaxis of infectious diseases and autoimmune diseases.

In other embodiments of the present invention a reporter gene is utilized either alone or in combination with a therapeutic gene. Examples of reporter genes include, but are not limited to genes encoding for fluorescent proteins, such as gfp, rfp, or bfp, enzymatic proteins like β-gal, or chemiluminescent proteins like luciferase.

Encompassed within the definition of “reporter gene” is a “biologically equivalent” therapeutic gene. Accordingly, sequences that have about 70% to about 99% homology of amino acids that are identical or functionally equivalent to the amino acid of the reporter gene will be sequences that are biologically functional equivalents provided the biological activity of the protein is maintained.

e. Nucleic Acids Encoding Antigens

The pharmaceutical compositions set forth herein may include a nucleic acid that encodes one or more antigens. For example, the therapeutic gene may encode antigens present in tumors, pathogens, or immune effectors involved in autoimmunity. These genes can be applied, for example, in formulations that would be applied in vaccinations for immune therapy or immune prophylaxis of neoplasias, infectious diseases and autoimmune diseases.

i. Tumor Antigens

In certain embodiments, the therapeutic nucleic acid encodes a tumor antigen. Tumor antigens are well-known to those of ordinary skill in the art. Examples include, but are not limited to, those described by Dalgleish (2004), Finn (2003), and Hellstrom and Helstrom (2003), each of which is herein incorporated by reference in its entirety. Other examples can be found on http://www.bioinfo.org.cn/hptaa/search.php, which is herein specifically incorporated by reference.

ii. Microorganism Antigens

In some embodiments, the nucleic acid encodes a microorganism antigen. The term “microorganism” includes viruses, bacteria, microscopic fungi, protozoa and other microscopic parasites. A “microorganism antigen” refers to a polypeptide that, when presented on the cell surface by antigen presenting cells (APCs), induces an immune response. This response may include a cytotoxic T cell response or the production of antibodies or both.

Examples of viruses from which microorganism antigens may be derived include: human herpes viruses (HHVs)-1 through 8; herpes B virus; HPV-16, 18, 31, 33, and 45; hepatitis viruses A, B, C, δ; poliovirus; rotavirus; influenza; lentiviruses; HTLV-1; HTLV-2; equine infectious anemia virus; eastern equine encephalitis virus; western equine encephalitis virus; venezuelan equine encephalitis virus; rift valley fever virus; West Nile virus; yellow fever virus; Crimean-Congo hemorrhagic fever virus; dengue virus; SARS coronavirus; small pox virus; monkey pox virus and/or the like.

Examples of viral microorganisms include, but are not limited to: retroviridae, flaviridae, coronaviridae, picornaviridae, togaviridae, rhabdoviridae, paramyxoviridae, orthomyxoviridae, bunyaviridae, arenaviridae, reoviridae, polyomaviridae, papillomaviridae, herpesviridae and hepadnaviridae.

Examples of retroviridae include lentiviruses such as HIV-1, HIV-2, SIV, FIV, Visna, CAEV, BIV and EIAV. Genes encoded by lentiviruses may include gag, pol, env, vif, vpr, vpu, nef, tat, vpx and rev. Other examples of retroviruses include alpha retroviruses such as avian leukosis virus, avian myeloblastosis virus, avian sarcoma virus, fujinami sarcoma virus and rous sarcoma virus. Genes encoded by alpha retroviruses may include gag, pol and env. Further examples of retroviruses include beta retroviruses such as jaagsiekte sheep retrovirus, langur virus, Mason-Pfizer monkey virus, mouse mammary tumor virus, simian retrovirus 1 and simian retrovirus 2. Genes encoded by beta retroviruses may include gag, pol, pro and env. Still further examples of retroviruses include delta retroviruses such as HTLV-1, HTLV-2, bovine leukemia virus, and baboon T cell leukemia virus. Genes encoded by delta retroviruses may include gag, pol, env, tax and rex. Still further examples of retrovirus include spumaviruses such as bovine, feline, equine, simian and human foamy viruses. Genes encoded by spumaviruses may include gag, pol, env, bel-1, bel-2 and bet.

Examples of flaviridae include but are not limited to: hepatitis C virus, mosquito borne yellow fever virus, dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, louping III virus, Powassan virus, Omsk hemorrhagic fever virus, the genus rubivirus (rubella virus) and the genus pestivirus (mucosal disease virus, hog cholera virus, border disease virus). Genes encoded by flaviviruses include the flavivirus polyprotein from which all flavivirus proteins are derived. Nucleic acid sequences encoding the flavivirus polyprotein may include sequences encoding the final processed flavivirus protein products such as C, prM, E, NS1, NS2A, NS2B, NS3, NS4A, NS4B and NS5.

Examples of coronaviridae include but are not limited to: human respiratory coronaviruses such as SARS and bovine coronaviruses. Genes encoded by coronaviridae may include pol, S, E, M and N.

Examples of picornaviridae include but are not limited to the genus Enterovirus (poliovirus, Coxsackie virus A and B, enteric cytopathic human orphan (ECHO) viruses, hepatitis A virus, simian enteroviruses, murine encephalomyelitis (ME) viruses, poliovirus muris, bovine enteroviruses, porcine enteroviruses, the genus cardiovirus (encephalomyocarditis virus (EMC), mengovirus), the genus rhinovirus (human rhinoviruses including at least 113 subtypes; other rhinoviruses) and the genus apthovirus (foot and mouth disease (FMDV). Genes encoded by picornaviridae may include the picornavirus polyprotein. Nucleic acid sequences encoding the picornavirus polyprotein may include sequences encoding the final processed picornavirus protein products such as VPg, VP0, VP3, VP1, 2A, 2B, 2C, 3A, 3B, 3C and 3D.

Examples of togaviridae include but are not limited to including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis Eastern equine encephalitis virus). Examples of genes encoded by togaviridae may include genes coding for nsP1, nsP2, nsP3 nsP4, C, E1 and E2.

Examples of rhabdoviridae include, but are not limited to: including the genus vesiculovirus (VSV), chandipura virus, Flanders-Hart Park virus) and the genus lyssavirus (rabies virus). Examples of genes encoded by rhabdoviridae may include N, P, M, G, and L.

Examples of filoviridae include Ebola viruses and Marburg virus. Examples of genes encoded by filoviruses may include NP, VP35, VP40, GP, VP35, VP24 and L. Examples of paramyxoviruses include, but are not limited to: including the genus paramyxovirus (parainfluenza virus type 1, sendai virus, hemadsorption virus, parainfluenza viruses types 2 to 5, Newcastle disease Virus, mumps virus), the genus morbillivirus (measles virus, subacute sclerosing panencephalitis virus, distemper virus, Rinderpest virus), the genus pneumovirus (respiratory syncytial virus (RSV), bovine respiratory syncytial virus and pneumonia virus of mice). the family paramyxoviridae, including the genus Paramyxovirus (Parainfluenza virus type 1, Sendai virus, hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice). Examples of genes encoded by paramyxoviridae may include N, P/C/V, P/C/V/R, M, F, HN, L, V/P, NS1, NS2, SH and M2.

Examples of orthomyxoviridae include influenza viruses. Examples of genes encoded by orthomyxoviridae may include PB1, PB2, PA, HA, NP, NA, M1, M2, NS1 and NS2.

Examples of bunyaviruses include, but are not limited to: the genus bunyvirus (bunyamwera and related viruses, California encephalitis group viruses), the genus phlebovirus (sandfly fever Sicilian virus, Rift Valley fever virus), the genus nairovirus (Crimean-Congo hemorrhagic fever virus, Nairobi sheep disease virus) and the genus uukuvirus (uukuniemi and related viruses). Examples of genes encoded by bunyaviruses may include N, G1, G2 and L.

Examples of arenaviruses include, but are not limited to: lymphocytic choriomeningitis virus, lassa fever virus, Argentine hemorrhagic fever virus, Bolivian hemorrhagic fever virus and Venezuelan hemorrhagic fever virus. Examples of genes encoded by arenaviruses may include NP, GPC, L and Z.

Examples of reoviruses include, but are not limited to: the genus orthoreovirus (multiple serotypes of both mammalian and avian retroviruses), the genus orbivirus (Bluetongue virus, Eugenangee virus, Kemerovo virus, African horse sickness virus, and Colorado Tick Fever virus) and the genus rotavirus (human rotavirus, Nebraska calf diarrhea virus, murine rotavirus, simian rotavirus, bovine or ovine rotavirus, avian rotavirus). Examples of genes encoded by reoviruses may include genome segments named for their corresponding protein products, such as VP1, VP2, VP3, VP4, NSP1, NSP3, NSP2, VP7, NSP4, NSP5 and NSP6.

Examples of polyomaviridae include, but are not limited to BK and JC viruses. Examples of genes encoded by polyomaviruses may include Agno, P2, VP3, VP2, VP1, large T and small t.

Examples of papillomaviridae include, but are not limited to: HPV-16 and HPV-18. Examples of genes encoded by papillomaviruses may include E1, E2, E3, E4, E5, E6, E7, E8, L1 and L2.

Examples of herpesviridae include, but are not limited to: Human Herpes Virus (HHV) 1, HHV2, HHV3, HHV4, HHV5, HHV6, HHV7 and HHV8. Examples of genes encoded by herpesviruses may include γ₇34.5, ORF P, ORFO, αO, U_{L}1 through U_{L}56, α4, α22, U_{S}2 through U_{S}12, Ori_{S}TU and LATU.

Examples of hepadnaviruses include but is not limited to hepatitis B virus. Examples of genes encoded by hepadnaviruses may include S, C, P and X.

Examples of fungi from which microorganism antigens may be derived include: histoplasma capsulatum; aspergillus; actinomyces; candida, streptomyces and/or the like.

Examples of protozoa or other microorganisms from which antigens may be derived include plasmodium falciparum, plasmodium vivax, plasmodium ovale, plasmodium malariae, and the like. Genes derived from plasmodium species may include PyCSP, MSP1, MSP4/5, Pvs25 and Pvs28.

Examples of bacteria from which microorganism antigens may be derived include: mycobacterium tuberculosis; yersinia pestis; rickettsia prowazekii; rickettsia rickettsii; francisella tularensis; bacillus anthracis; helicobacter pylori; salmonella typhi; borrelia burgdorferi; streptococcus mutans; and/or the like. Genes derived from mycobacterium tuberculosis may include 85A, 85B, 85C and ESAT-6. Genes derived from yersinia pestis may include lcrV and caf1. Genes derived from rickettsia species may include ospA, invA, ompA, ompB, virB, cap, tlyA and tlyC. Genes derived from francisella tularensis may include nucleoside diphosphate kinase, isocitrate dehydrogenase, Hfq and ClpB. Genes derived from bacillus anthracis may include PA, BclA and LF. Genes derived from helicobacter pylori may include hpaA, UreB, hspA, hspB, hsp60, VacA, and cagE. Genes derived from salmonella typhi may include mpC, aroC, aroD, htrA and CS6. Genes derived from borrelia burgdorferi may include OspC.

Examples of fungi from which microorganism antigens may be derived include: hitoplasma; ciccidis; immitis; aspargillus; actinomyces; blastomyces; candida, streptomyces and/or the like.

Examples of protozoa or other microorganisms from which antigens may be derived include: plasmodium falciparum; plasmodium vivax; plasmodium ovale; plasmodium malariae; giadaria intestinalis and/or the like.

The microorganism antigen may be a glucosyltransferases derived from Streptococci mutans. The glucosyltransferases mediate the accumulation of S. mutans on the surface of teeth. Inactivation of glucosyltransferase has been demonstrated to cause a reduction in dental caries (Devulapalle and Mooser, 2001).

Another example an antigen derived from Streptococci mutans is PAc protein. PAc is a 190-kDa surface protein antigen involved in the colonization of Streptococci mutans, which mediates the initial adherence of this organism to tooth surfaces. Recently, it has been reported that in vivo administration of plasmid DNA encoding a fusion protein of amino acid residues 1185-1475 encoded by the glucosyltransferase-B of S. mutans, and amino acid residues 222-965 encoded by the PAc gene of S. mutans elicited an immune response against these respective gene products (Guo et al., 2004).

f. Nucleic Acids Encoding Antibodies

The nucleic acids set forth herein may encode an antibody. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

In certain embodiments of the present invention, the nucleic acid of the pharmaceutical compositions set forth herein encodes a single chain antibody. Single-chain antibodies are described in U.S. Pat. Nos. 4,946,778 and 5,888,773, each of which are hereby incorporated by reference.

g. Ribozymes

In certain embodiments of the present invention, the nucleic acid of the pharmaceutical compositions set forth herein encodes or comprises a ribozyme. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

h. RNAi

In certain embodiments of the present invention, the therapeutic nucleic acid of the pharmaceutical compositions set forth herein is an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

One of ordinary skill in the art of RNAi understands that there are additional types of RNAi including but not limited to microRNA that may also be similarly employed in the present invention. microRNA is described in Du and Zamore, 2005, which is herein specifically incorporated by reference in its entirety.

The endoribonuclease Dicer is known to produce two types of small regulatory RNAs that regulate gene expression: small interfering RNAs (siRNAs) and microRNAs (miRNAs) (Bernstein et al., 2001; Grishok et al., 2001; Hutvagner et al., 2001; Ketting et al., 2001; Knight and Bass, 2001). In animals, siRNAs direct target mRNA cleavage (Elbashir et al., 2001), whereas miRNAs block target mRNA translation (Reinhart et al., 2000; Brennecke et al., 2003; Xu et al., 2003). Recent data suggest that both siRNAs and miRNAs incorporate into similar perhaps even identical protein complexes, and that a critical determinant of mRNA destruction versus translation regulation is the degree of sequence complementary between the small RNA and its mRNA target (Hutvagner and Zamore, 2002; Mourelatos et al., 2002; Zeng et al., 2002; Doench et al., 2003; Saxena et al., 2003). Many known miRNA sequences and their position in genomes or chromosomes can be found in http://www.sanger.ac.uk/Software/Rfam/mirna/help/summary.shtml.

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy)thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

U.S. Patent App. 20050203047 reports of a method of modulating gene expression through RNA interference by incorporating a siRNA or miRNA sequence into a transfer RNA (tRNA) encoding sequence. The tRNA containing the siRNA or miRNA sequence may be incorporated into a nucleic acid expression construct so that this sequence is spliced from the expressed tRNA. The siRNA or miRNA sequence may be positioned within an intron associated with an unprocessed tRNA transcript, or may be positioned at either end of the tRNA transcript.

i. Other Therapeutic Nucleic Acids

Other examples of therapeutic nucleic acids include oligonucleotides that include a CpG domain (“CpG oligonucleotides”). It has been demonstrated that bacterial DNA has a direct immunostimulatory effect on peripheral blood mononuclear cells in vitro. (Messina et al., 1991). Such effects include proliferation of B cells and increased immunoglobulin Ig secretion. (Krieg et al., 1995) Additionally, these effects include Th1 cytokine secretion, including IL-12, via activation of monocytes, macrophages and dendritic cells. (Klinman, et al., 1996; Halpern et al., 1996; Cowdery et al., 1996) The secreted Th1 cytokines stimulate natural killer (NK) cells to secrete γ-interferon and to have increased lytic activity. (Klinman et al., 1996, supra; Cowdery et al., 1996, supra; Yamamoto et al., 1992) These stimulatory effects are often the result of the presence of unmethylated CpG dinucleotides in a particular sequence context (CpG-S) (Krieg et al., 1995).

B cell activation by CpG-S sequences is T cell independent and antigen non-specific. Nevertheless, CpG-S sequences have strong synergy with signals delivered through the B cell antigen receptor. This interaction with the B cell antigen receptor does promote antigen specific immune responses, suggesting the desirability of CpG sequences as an immune stimulation adjuvant.

CpG-S sequences contain a cytosine-guanine dinucleotide and generally are between 2 to 100 base pairs in size. A consensus CpG-S sequence is represented by the formula: ^{5′}X₁X₂CGX₃X₄^{3′}, where X₁, X₂, X₃ and X₄ are nucleotides and a GCG trinucleotide sequence is not present at or near the 5′ and 3′ ends. Examples of CpG-S sequences include GACGTT, AGCGTT, AACGCT, GTCGTT and AACGAT.

Conversely, some microorganisms contain CpG sequences which appear to be immune neutralizing, such as adenovirus serotype 2. In these viruses, most CpG sequences are found in clusters of direct repeats or with a C on the 5′ side or a G on the 3′ side. It appears that such CpG sequences are immune-neutralizing (CpG-N) in that they block the Th1-type immune activation by CpG-S sequences in vitro. Likewise, when CpG-N and CpG-S sequences are administered with antigen, the antigen-specific immune response is blunted compared to that with CpG-S sequences alone. When CpG-N sequences alone are administered in vivo with an antigen, a Th2-like antigen-specific immune response develops.

GpG-N sequences also contain a cytosine-guanine dinucleotide and generally are between 2 to 100 base pairs in length. A consensus CpG-N sequence is represented by the formula: ^{5′}GCGXNGCG^{3′}, where X is any nucleotide and n is in the range of 0-50.

Accordingly, nucleotide sequences in a nucleic acid construct may be manipulated to increase the number of CpG-S sequences. Such constructs may also be manipulated to decrease the number of CpG-N sequences. For instance, those of ordinary skill in the art may choose to utilize site directed mutagenesis to produce a desired nucleic acid sequence with one or more CpG motifs. Alternatively, particular CpG sequences can be synthesized and inserted into the nucleic acid construct. Non-limiting examples are provided in U.S. Pat. Nos. 5,889,136, 4,415,723, and 4,458,066, expressly incorporated herein by reference,

U.S. Pat. No. 6,194,388 and U.S. Pat. No. 6,207,646 suggest that GpG oligonucleotides for use in immune stimulation may stabilized to provide resistance to degradation. Both of these texts are incorporated herein in their entirety by reference. The stabilization process contemplated in these references is accomplished via phosphate backbone modifications. A preferred stabilized oligonucleotide has a phosphorothioate modified backbone. The pharmacokinetics of phosphorothioate oligonucleotides demonstrate a systemic half life of 48 hours in rodents (Agrawal et al., 1991). These phosphorothioates may be synthesized using automated techniques employing either phosphoramidate or H phosphonate chemistries. Aryl- and alkyl-phosphonates can be made as described in U.S. Pat. No. 4,469,863; and alkylphosphotriesters in which the charged oxygen moiety is alkylated is described in U.S. Pat. No. 5,023,243, each of which is herein specifically incorporated by reference in their entirety. Other methods for making DNA backbone modifications and substitutions have also been described (Uhlmann, E. and Peyman, A., 1990, and Goodchild, 1990).

U.S. Pat. No. 6,206,646 reports that unmethylated CpG containing nucleic acid molecules having a phosphorothioate backbone have been found to preferentially activate B-cell activity, while unmethylated CpG containing nucleic acid molecules having a phosphodiester backbone have been found to preferentially activate macrophages, dendritic cells, monocytes and NK cells. The modification preferentially occurs at or near the 5′ and/or 3′ end of the nucleic acid molecule.

U.S. Pat. No. 6,339,068 reports that DNA vectors for immune stimulation immune can be improved by removal of CpG-N sequences and further improved by the addition of CpG-S sequences. In addition, for high and long-lasting levels of expression, the optimized vector should preferably include a promoter/enhancer, which is not down-regulated by the cytokines induced by the immunostimulatory CpG sequences. Also reported was a method of generating such a plasmid based DNA vector encoding the hepatitis B surface antigen gene. However, the same reference indicates that CpG-S sequences must be administered at the same time or at the same place (i.e. on the antigen encoding plasmid) for an immune stimulation effect. Yet, it does not appear that the modification must be within the antigen sequence itself.

U.S. Pat. No. 6,399,068 also reports that NFκB is a mediator of the CpG effect. For instance, within 15 minutes of treating B cells or monocytes with CpG sequences, the level of NFκB binding activity is increased, while the same cell types treated with DNA not containing these sequences shows change. The reference also reports that inhibition of NFκB activation blocks lymphocyte stimulation by CpG sequences. Additionally, CpG DNA causes a rapid induction of the production of reactive oxygen species B cells and monocytic cells as detected by the sensitive fluorescent dye dihydrorhodamine 123 as described in Royall and Ischiropoulos, 1993. Further it was reported that the generation of reactive oxygen species following treatment of B cells with CpG DNA requires that the DNA undergo an acidification step in the endosomes. Based on electrophoretic mobility shift assays (EMSA) with 5′ radioactively labeled oligonucleotides with or without CpG motifs, a band was found which appears to represent a protein binding specifically to a single stranded oligonucleotide having a CpG sequence. This binding was reported to be blocked if oligonucleotides containing NFκB binding sites was added.

Any other nucleic acid that is contemplated to be of benefit in the treatment or prevention of a disease or health-related condition that is not specifically set forth herein is also contemplated for inclusion in the compositions and methods of the present invention. The therapeutic nucleic acids set forth herein may further comprise or encode a reporter sequence. Reporter sequences are discussed in greater detail below.

3. Diagnostic Nucleic Acids

The pharmaceutical compositions of the present invention may include a nucleic acid that is a diagnostic nucleic acid. A “diagnostic nucleic acid” is a nucleic acid that can be applied in the diagnosis of a disease or health-related condition. Also included in the definition of “diagnostic nucleic acid” is a nucleic acid sequence that encodes one or more reporter proteins. A “reporter protein” refers to an amino acid sequence that, when present in a cell or tissue, is detectable and distinguishable from other genetic sequences or encoded polypeptides present in cells. In some embodiments, a therapeutic gene may be fused to the reporter or be produced as a separate protein. For example, the gene of interest and reporter may be induced by separate promoters in separate delivery vehicles by co-transfection (co-infection) or by separate promoters in the same delivery vehicle. In addition, the two genes may be linked to the same promoter by, for example, an internal ribosome entry site, or a bi-directional promoter. Using such techniques, expression of the gene of interest and reporter correlate. Thus, one may gauge the location, amount, and duration of expression of a gene of interest. The gene of interest may, for example, be an anti-cancer gene, such as a tumor suppressor gene or pro-apoptotic gene.

Because cells can be transfected with reporter genes, the reporter may be used to follow cell trafficking. For example, in vitro, specific cells may be transfected with a reporter and then returned to an animal to assess homing. In an experimental autoimmune encephalomyelitis model for multiple sclerosis, Costa et al. (2001) transferred myelin basic protein-specific CD4+ T cells that were transduced to express IL-12 p40 and luciferase. In vivo, luciferase was used to demonstrate trafficking to the central nervous system. In addition, IL-12 p40 inhibited inflammation. In another system, using positron emission tomography (PET), Koehne et al. (2003) demonstrated in vivo that Epstein-Barr virus (EBV)-specific T cells expressing herpes simplex virus-1 thymidine kinase (HSV-TK) selectively traffic to EBV+ tumors expressing the T cells' restricting HLA allele. Furthermore, these T cells retain their capacity to eliminate targeted tumors. Capitalizing on sequential imaging, Dubey et al (2003) demonstrated antigen specific localization of T cells expressing HSV-TK to tumors induced by murine sarcoma virus/Moloney murine leukemia virus (M-MSV/M-MuLV). Tissue specific promoters may also be used to assess differentiation, for example, a stem cell differentiating or fusing with a liver cell and taking up the characteristics of the differentiated cell such as activation of the surfactant promoter in type II pneumocytes.

Preferably, a reporter sequence encodes a protein that is readily detectable either by its presence, its association with a detectable moiety or by its activity that results in the generation of a detectable signal. In certain aspects, a detectable moiety may include a radionuclide, a fluorophore, a luminophore, a microparticle, a microsphere, an enzyme, an enzyme substrate, a polypeptide, a polynucleotide, a nanoparticle, and/or a nanosphere, all of which may be coupled to an antibody or a ligand that recognizes and/or interacts with a reporter.

In various embodiments, a nucleic acid sequence of the invention comprises a reporter nucleic acid sequence or encodes a product that gives rise to a detectable polypeptide. A reporter protein is capable of directly or indirectly generating a detectable signal. Generally, although not necessarily, the reporter gene includes a nucleic acid sequence and/or encodes a detectable polypeptide that are not otherwise produced by the cells. Many reporter genes have been described, and some are commercially available for the study of gene regulation (e.g., Alam and Cook, 1990, the disclosure of which is incorporated herein by reference). Signals that may be detected include, but are not limited to color, fluorescence, luminescence, isotopic or radioisotopic signals, cell surface tags, cell viability, relief of a cell nutritional requirement, cell growth and drug resistance. Reporter sequences include, but are not limited to, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, G-protein coupled receptors (GPCRs), somatostatin receptors, CD2, CD4, CD8, the influenza hemagglutinin protein, symporters (such as NIS) and others well known in the art, to which high affinity antibodies or ligands directed thereto exist or can be produced by conventional means, and fusion proteins comprising a membrane bound protein appropriately fused to an antigen tag domain from, among others, hemagglutinin or Myc. Kundra et al., 2002, demonstrated noninvasive monitoring of somatostatin receptor type 2 chimeric gene transfer in vitro and in vivo using biodistribution studies and gamma camera imaging.

In some embodiments, a reporter sequence encodes a fluorescent protein. Examples of fluorescent proteins which may be used in accord with the invention include green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In various embodiments, the desired level of expression of at least one of the reporter sequence is an increase, a decrease, or no change in the level of expression of the reporter sequence as compared to the basal transcription level of the diagnostic nucleic acid. In a particular embodiment, the desired level of expression of one of the reporter sequences is an increase in the level of expression of the reporter sequence as compared to the basal transcription level of the reporter sequence.

In various embodiments, the reporter sequence encodes unique detectable proteins which can be analyzed independently, simultaneously, or independently and simultaneously. In other embodiments, the host cell may be a eukaryotic cell or a prokaryotic cell. Exemplary eukaryotic cells include yeast and mammalian cells. Mammalian cells include human cells and various cells displaying a pathologic phenotype, such as cancer cells.

For example, some reporter proteins induce color changes in cells that can be readily observed under visible and/or ultraviolet light. The reporter protein can be any reporter protein known to those of ordinary skill in the art. Examples include gfp, rfp, bfp and luciferase.

Nucleic acids encoding reporter proteins include DNAs, cRNAs, mRNAs, and subsequences thereof encoding active fragments of the respective reporter amino acid sequence, as well as vectors comprising these sequences.

Exemplary methods of imaging of reporter proteins includes gamma camera imaging, CT, MRI, PET, SPECT, optical imaging, and ultrasound. In some embodiments, the diagnostic nucleic acid is suitable for imaging using more than one modality, such as CT and MRI, PET and SPECT, and so forth.

Additional information pertaining to examples of reporters in imaging are set forth in Kumar, 2005; Kundra et al., 2005; and Kundra et al., 2002, each of which is herein specifically incorporated by reference in its entirety.

4. Antisense Constructs

In some embodiments set forth herein, the nucleic acid encodes an antisense construct. Antisense methodology takes advantage of the fact that nucleic acids tend to pair with “complementary” sequences.” By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

### B. EXPRESSION CASSETTES

1. Overview

In certain embodiments of the present invention, the pharmaceutical compositions and methods set forth herein involve therapeutic or diagnostic nucleic acids, wherein the nucleic acid is comprised in an “expression cassette.” Throughout this application, the term “expression cassette” is meant to include any type of genetic construct containing a nucleic acid coding for a gene product in which part or all of the nucleic acid encoding sequence is capable of being transcribed.

2. Promoters and Enhancers

In order for the expression cassette to effect expression of a transcript, the nucleic acid encoding the diagnostic or therapeutic gene will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a cell in any cell in a subject is contemplated as a promoter that can be applied in the methods and compositions of the present invention. As discussed elsewhere, a subject can be any subject, including a human and another mammal, such as a mouse or laboratory animal. One of ordinary skill in the art would be familiar with the numerous types of promoters that can be applied in the present methods and compositions. In certain embodiments, for example, the promoter is a constitutive promoter, an inducible promoter, or a repressible promoter. The promoter can also be a tissue selective promoter. A tissue selective promoter is defined herein to refer to any promoter which is relatively more active in certain tissue types compared to other tissue types. Thus, for example, a liver-specific promoter would be a promoter which is more active in liver compared to other tissues in the body. One type of tissue-selective promoter is a tumor selective promoter. A tumor selective promoter is defined herein to refer to a promoter which is more active in tumor tissue compared to other tissue types. There may be some function in other tissue types, but the promoter is relatively more active in tumor tissue compared to other tissue types. Examples of tumor selective promoters include the hTERT promoter, the CEA promoter, the PSA promoter, the probasin promoter, the ARR2PB promoter, and the AFP promoter.

The promoter may be one which is active in a particular target cell. For instance, where the target cell is a keratinocyte, the promoter will be one which has activity in a keratinocyte. Similarly, where the cell is an epithelial cell, skin cell, mucosal cell or any other cell that can undergo transformation by a papillomavirus, the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosine (melanoma), alpha-fetoprotein and albumin (liver tumors), CC10 (lung tumors) and prostate-specific antigen (prostate tumor) will permit tissue-specific expression of the therapeutic nucleic acids set forth herein. Table 2 lists additional examples of promoters/elements which may be employed, in the context of the present invention, to regulate the expression of the anti-cancer genes. This list is not intended to be exhaustive of all the possible promoter and enhancer elements, but, merely, to be exemplary thereof.

TABLE 2

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and continuous, often seeming to have very similar modular organization.

Additionally, any promoter/enhancer combination (as per the Eukaryotic Promoter Data Base EPDB) could also be used to drive expression of a diagnostic or therapeutic gene. Use of a T3, T7, or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional expression vector.

Further selection of a promoter that is regulated in response to specific physiologic signals can permit inducible expression of a construct. For example, with the polynucleotide under the control of the human PAI-1 promoter, expression is inducible by tumor necrosis factor. Table 3 provides examples of inducible elements, which are regions of a nucleic acid sequence that can be activated in response to a specific stimulus.

TABLE 3

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Ponta et al., 1985; Sakai et |
| | | al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

3. Reporter Genes

In certain embodiments of the invention, the delivery of an expression cassette may be identified in vitro or in vivo by including a reporter gene in the expression vector. The reporter gene would result in an identifiable change to the transfected cell permitting easy identification of expression. Usually the inclusion of a drug selection marker aids in cloning and in the selection of transformants. Alternatively, enzymes such as β-galactosidase (β-gal) herpes simplex virus thymidine kinase (tk) (eukaryotic) or chloramphenical acetyltransferase (CAT)(prokaryotic) may be employed. Fluorescent and chemiluminescent markers are contemplated as well. Immunologic markers can also be employed. The selectable reporter gene employed is not believed to be important, so long as it is capable of being expressed along with the therapeutic nucleic acid. Further examples of selectable reporter genes are well known to one of skill in the art.

4. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

5. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819). One of ordinary skill in the art would be familiar with the application of IRES in gene therapy.

6. Multiple Cloning Sites

Expression cassettes can include a multiple cloning site (MCS), which is a nucleic acid region that contains multiple restriction enzyme sites, any of which can be used in conjunction with standard recombinant technology to digest the vector. See Carbonelli et al. (1999); Levenson et al. (1998); Cocea (1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997).

7. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

8. Other Expression Cassette Components

In certain embodiments of the present invention, the expression cassette comprises a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or Bluescript™ plasmid series.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the invention, a treated cell may be identified in vitro or in vivo by including a reporter gene in the expression vector. Such reporter genes would confer an identifiable change to the cell permitting easy identification of cells containing the expression vector. Generally, a selectable reporter is one that confers a property that allows for selection. A positive selectable reporter is one in which the presence of the reporter gene allows for its selection, while a negative selectable reporter is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of reporters including screenable reporters such as GFP or luciferase, are also contemplated. Alternatively, screenable enzymes such as herpes simplex virus thymidine kinase (tk) or chloramphenicol acetyltransferase (CAT) may be utilized. One of skill in the art would also know how to employ immunologic reporters, possibly in conjunction with FACS analysis. The marker used is not believed to be important, so long as it is capable of being expressed simultaneously with the nucleic acid encoding a gene product. Further examples of selectable and screenable reporters are well known to one of skill in the art.

In certain embodiments of the invention, it is contemplated that the reporter gene will be operatively linked to a tissue specific promoter such that the reporter gene product, such as GFP will be expressed only in cells of a contemplated tissue type. For example, the gfp reporter gene may be operatively linked to an hTERT promoter within a replication selective adenoviral vector, thereby detecting hyperproliferative lesions with telomerase specific GFP expression (Umeoka et al., 2004.)

### C. VIRAL VECTORS

A viral vector is a virus that can transfer genetic material from one location to another, such as from the point of application to a target cell of interest. In certain embodiments of the present invention, the nucleic acids of the compositions set forth herein is a “naked” nucleic acid sequence, which is not comprised in a viral vector or delivery agent, such as a lipid or liposome. In other embodiments of the present invention, however, the nucleic acid is comprised in a viral vector. One of ordinary skill in the art would be familiar with the various types of viruses that are available for use as vectors for gene delivery to a target cell of interest. Each of these is contemplated as a vector in the present invention. Exemplary vectors are discussed below.

1. Viral Vectors

A “viral vector” is meant to include those constructs containing viral sequences sufficient to (a) support packaging of an expression cassette comprising the therapeutic nucleic acid sequences and (b) to ultimately express a recombinant gene construct that has been cloned therein.

a. Adenoviral Vectors

The pharmaceutical compositions and methods of the present invention may involve expression constructs of the therapeutic nucleic acids comprised in adenoviral vectors for delivery of the nucleic acid. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both nondividing an dividing cells and can be produced in large quantities. In many of the clinical trials for cancer, local intratumor injections have been used to introduce the vectors into sites of disease because current vectors do not have a mechanism for preferential delivery to tumor. In vivo experiments have demonstrated that administration of adenovirus vectors systemically resulted in expression in the oral mucosa (Clayman et al., 1995). Topical application of Ad-βgal and Ad-p53-FLAG on organotypic raft cultures has demonstrated effective gene transduction and deep cell layer penetration through multiple cell layers (Eicher et al., 1996). Therefore, gene transfer strategy using the adenoviral vector is potentially feasible in patients at risk for lesions and malignancies involving genetic alterations in p53.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

c. AAV Vectors

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte and Carter, 1995).

d. Herpesvirus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of α genes, the first set of genes to be expressed after infection, is enhanced by the virion protein number 16, or α-transinducing factor (Post et al., 1981; Batterson and Roizman, 1983). The expression of β genes requires functional a gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. A virulent variants of HSV have been developed and are readily available for use in gene therapy contexts (U.S. Pat. No. 5,672,344).

e. Vaccinia Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

f. Oncolytic Viral Vectors

Oncolytic viruses are also contemplated as vectors in the present invention. Oncolytic viruses are defined herein to generally refer to viruses that kill tumor or cancer cells more often than they kill normal cells. Exemplary oncolytic viruses include adenoviruses which overexpress ADP. These viruses are discussed in detail in U.S. Patent Application Pub. No. 20040213764, U.S. Patent Application Pub. No. 20020028785, and U.S. patent application Ser. No. 09/351,778, each of which is specifically incorporated by reference in its entirety into this section of the application and all other sections of the application. Exemplary oncolytic viruses are discussed elsewhere in this specification. One of ordinary skill in the art would be familiar with other oncolytic viruses that can be applied in the pharmaceutical compositions and methods of the present invention.

g. Other Viral Vectors

Other viral vectors that may be employed as vectors in the present invention include those viral vectors that can be applied in vaccines, or in dual vaccine and immunotherapy applications. Viral vectors, and techniques for vaccination and immunotherapy using viral vectors, are described in greater detail in PCT application WO0333029, WO0208436, WO0231168, and WO0285287, each of which is specifically incorporated by reference in its entirely for this section of the application and all other sections of this application. Additional vectors that can be applied in the techniques for vaccination and dual immunotherapy/vaccination include those oncolytic viruses set forth above.

Other viral vectors also include baculovirus vectors, parvovirus vectors, picomavirus vectors, alphavirus vectors, semiliki forest virus vectors, Sindbis virus vectors, lentivirus vectors, and retroviral vectors. Vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Other viral vectors for application in the compositions and methods of the present invention include those vectors set forth in Tang et al., 2004, which is herein specifically incorporated by reference in its entirety for this section of the application and all other sections of the application.

i. Gene Delivery Using Modified Viruses

A diagnostic or therapeutic nucleic acid may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### D. DELIVERY AGENTS

In certain embodiments of the present invention, the nucleic acid encoding an amino acid sequence may further comprise a delivery agent. A delivery agent is defined herein to refer to any agent or substance, other than a viral vector, that facilitates the delivery of the nucleic acid to a target cell of interest. Exemplary delivery agents include lipids and lipid formulations, including liposomes. In certain embodiments, the lipid is comprised in nanoparticles. A nanoparticle is herein defined as a submicron particle. For example, the nanoparticle may have a diameter of from about 1 to about 500 nanometers. The particle can be composed of any material or compound. In the context of the present invention, for example, a “nanoparticle” may include certain liposomes that have a diameter of from about 1 to about 500 nanometers.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome.” This formulation is reported to “sandwich” DNA between an invaginated bi-layer or ‘vase’ structure. Beneficial characteristics of these liposomes include a positive p, colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

The liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

In addition, one of ordinary skill in the art is aware of other nanoparticle formulations suitable for gene delivery. Examples include those nanoparticle formulations described by Bianco (2004), Doerr (2005), and Lang et al. (2005), each of which is herein specifically incorporated by reference in its entirety.

### E. THERAPIES

1. Definitions

A “therapeutic nucleic acid” is defined herein to refer to a nucleic acid that is known or suspected to be of benefit in the treatment or prevention of a disease or health-related condition. Contemplated within the definition of “therapeutic nucleic acid” is a nucleic acid that encodes a protein or polypeptide that is known or suspected to be of benefit in the treatment of a disease or health-related condition, as well as nucleic acids that more directly, such as a ribozyme. Therapeutic nucleic acids may also be nucleic acid that transcribe a nucleic acid that is known or suspected to be of benefit in the treatment of a disease or health-related condition (e.g., a nucleic acid transcribing a ribozyme).

The term “therapeutic” or “therapy” as used throughout this application refers to anything that is known or suspected to promote or enhance the well-being of the subject with respect to a disease or health-related condition. Thus, a “therapeutic nucleic acid” is a nucleic acid that is known or suspected to promote or enhance the well-being of the subject with respect to a disease or health-related condition. A list of nonexhaustive examples of such therapeutic benefit includes extension of the subject's life by any period of time, or decrease or delay in the development of the disease. In the case of cancer, therapeutic benefit includes decrease in hyperproliferation, reduction in tumor growth, delay of metastases or reduction in number of metastases, reduction in cancer cell or tumor cell proliferation rate, decrease or delay in progression of neoplastic development from a premalignant condition, and a decrease in pain to the subject that can be attributed to the subject's condition.

A “disease” is defined as a pathological condition of a body part, an organ, or a system resulting from any cause, such as infection, genetic defect, or environmental stress.

A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. In certain embodiments of the present invention, the methods involving delivery of a nucleic acid encoding a therapeutic protein to prevent a disease or health-related condition in a subject. An amount of a pharmaceutical composition that is suitable to prevent a disease or health-related condition is an amount that is known or suspected of blocking the onset of the disease or health-related condition.

“Diagnostic” or “diagnosis” as used throughout this application refers to anything that is known or suspected to be of benefit in identifying the presence or absence of a disease or health-related condition in a subject. Also included in this definition is anything that is known or suspected to be of benefit in the identification of subjects at risk of developing a particular disease or health-related condition. Thus, a diagnostic nucleic acid is a nucleic acid that is known or suspected to be of benefit in identifying the presence or absence of a disease or health-related condition, or that is known or suspected to be of benefit in identifying a subject at risk of developing a particular disease or health-related condition. For example, the diagnostic nucleic acid may be a nucleic acid that encodes a reporter protein that is detectable. Such a protein, for example, may find application in imaging modalities.

2. Diseases to be Diagnosed, Prevented or Treated

The present invention contemplates methods to detect, prevent, inhibit, or treat a disease in a subject by administration of a nucleic acid encoding an amino acid sequence capable of preventing or inhibiting disease in a subject. As set forth above, any nucleic acid sequence that can be applied or administered to a subject for the purpose of detecting, preventing, or inhibiting, or treating a disease is contemplated for inclusion in the pharmaceutical compositions set forth herein.

In certain embodiments, the disease may be a hyperproliferative disease that can affect a subject that would be amenable to detection, therapy, or prevention through administration of a nucleic acid sequence to the subject. For example, the disease may be a hyperproliferative disease. A hyperproliferative disease is a disease associated with the abnormal growth or multiplication of cells. The hyperproliferative disease may be a disease that manifests as lesions in a subject. Exemplary hyperproliferative lesions include the following: Squamous cell carcinoma, basal cell carcinoma, adenoma, adenocarcinoma, linitis plastica, insulinoma, glucagonoma, gastrinoma, vipoma, cholangiocarcinoma, hepatocellular carcinoma, adenoid cystic carcinoma, carcinoid tumor, prolactinoma, oncocytoma, hurthle cell adenoma, renal cell carcinoma, endometrioid adenoma, cystadenoma, pseudomyxoma peritonei, Warthin's tumor, thymoma, thecoma, granulosa cell tumor, arrhenoblastoma, Sertoli-Leydig cell tumor, paraganglioma, pheochromocytoma, glomus tumor, melanoma, soft tissue sarcoma, desmoplastic small round cell tumor, fibroma, fibrosarcoma, myxoma, lipoma, liposarcoma, leiomyoma, leiomyosarcoma, myoma, myosarcoma, rhabdomyoma, rhabdomyosarcoma, pleomorphic adenoma, nephroblastoma, brenner tumor, synovial sarcoma, mesothelioma, dysgerminoma, germ cell tumors, embryonal carcinoma, yolk sac tumor, teratomas, dermoid cysts, choriocarcinoma, mesonephromas, hemangioma, angioma, hemangiosarcoma, angiosarcoma, hemangioendothelioma, hemangioendothelioma, Kaposi's sarcoma, hemangiopericytoma, lymphangioma, cystic lymphangioma, osteoma, osteosarcoma, osteochondroma, cartilaginous exostosis, chondroma, chondrosarcoma, giant cell tumors, Ewing's sarcoma, odontogenic tumors, cementoblastoma, ameloblastoma, craniopharyngioma gliomas mixed oligoastrocytomas, ependymoma, astrocytomas, glioblastomas, oligodendrogliomas, neuroepitheliomatous neoplasms, neuroblastoma, retinoblastoma, meningiomas, neurofibroma, neurofibromatosis, schwannoma, neurinoma, neuromas, granular cell tumors, alveolar soft part sarcomas, lymphomas, non-Hodgkin's lymphoma, lymphosarcoma, Hodgkin's disease, small lymphocytic lymphoma, lymphoplasmacytic lymphoma, mantle cell lymphoma, primary effusion lymphoma, mediastinal (thymic) large cell lymphoma, diffuse large B-cell lymphoma, intravascular large B-cell lymphoma, Burkitt lymphoma, splenic marginal zone lymphoma, follicular lymphoma, extranodal marginal zone B-cell lymphoma of mucosa-associated lymphoid tissue (MALT-lymphoma), nodal marginal zone B-cell lymphoma, mycosis fungoides, Sezary syndrome, peripheral T-cell lymphoma, angioimmunoblastic T-cell lymphoma, subcutaneous panniculitis-like T-cell lymphoma, anaplastic large cell lymphoma, hepatosplenic T-cell lymphoma, enteropathy type T-cell lymphoma, lymphomatoid papulosis, primary cutaneous anaplastic large cell lymphoma, extranodal NK/T cell lymphoma, blastic NK cell lymphoma, plasmacytoma, multiple myeloma, mastocytoma, mast cell sarcoma, mastocytosis, mast cell leukemia, langerhans cell histiocytosis, histiocytic sarcoma, langerhans cell sarcoma dendritic cell sarcoma, follicular dendritic cell sarcoma, Waldenstrom macroglobulinemia, lymphomatoid granulomatosis, acute leukemia, lymphocytic leukemia, acute lymphoblastic leukemia, acute lymphocytic leukemia, chronic lymphocytic leukemia, adult T-cell leukemia/lymphoma, plasma cell leukemia, T-cell large granular lymphocytic leukemia, B-cell prolymphocytic leukemia, T-cell prolymphocytic leukemia, precursor B lymphoblastic leukemia, precursor T lymphoblastic leukemia, acute erythroid leukemia, lymphosarcoma cell leukemia, myeloid leukemia, myelogenous leukemia, acute myelogenous leukemia, chronic myelogenous leukemia, acute promyelocytic leukemia, acute promyelocytic leukemia, acute myelomonocytic leukemia, basophilic leukemia, eosinophilic leukemia, acute basophilic leukemia, acute myeloid leukemia, chronic myelogenous leukemia, monocytic leukemia, acute monoblastic and monocytic leukemia, acute megakaryoblastic leukemia, acute myeloid leukemia and myelodysplastic syndrome, chloroma or myeloid sarcoma, acute panmyelosis with myelofibrosis, hairy cell leukemia, juvenile myelomonocytic leukemia, aggressive NK cell leukemia, polycythemia vera, myeloproliferative disease, chronic idiopathic myelofibrosis, essential thrombocytemia, chronic neutrophilic leukemia, chronic eosinophilic leukemia/hypereosinophilic syndrome, post-transplant lymphoproliferative disorder, chronic myeloproliferative disease, myelodysplastic/myeloproliferative diseases, chronic myelomonocytic leukemia and myelodysplastic syndrome. In certain embodiments, the hyperproliferative lesion is a disease that can affect the mouth of a subject. Examples include leukoplakia, squamous cell hyperplastic lesions, premalignant epithelial lesions, intraepithelial neoplastic lesions, focal epithelial hyperplasia, and squamous carcinoma lesion.

In certain other embodiments, the hyperproliferative lesion is a disease that can affect the skin of a subject. Examples include squamous cell carcinoma, basal cell carcinoma, melanoma, papillomas (warts), and psoriasis. Treatment of. carcinomas related to viruses is also contemplated, including but not limited to cancers of the head and neck. The lesion may include cells such as keratinocytes, epithelial cells, skin cells, and mucosal cells. The disease may also be a disease that affects the lung mucosa.

The disease may be a precancerous lesion, such as leukoplakia of the oral cavity or actinic keratosis of the skin.

Other examples of diseases to be treated or prevented include infectious diseases and inflammatory diseases, such as autoimmune diseases. The methods and compositions of the present invention can be applied in to deliver an antigen that can be applied in immune therapy or immune prophylaxis of a disease. Other exemplary diseases include wounds, burns, skin ulcers, kyphosis, dermatological conditions (reviewed in Burns et al., 2004), dental disease such as gingivitis (reviewed in Neville et al., 2001), and ocular disease (reviewed in Yanoff et al., 2003). Gene therapy of wounds is reviewed in Eriksson and Vranckx, 2004; Atiyeh et al., 2005; Ferguson and O'Kane, 2004; Waller et al., 2004; Simon et al., 2004; and Bok and Bok, 2004, each of which is specifically incorporated by reference in their entirety herein. One of ordinary skill in the art would be familiar with the many disease entities that would be amenable to prevention or treatment using the pharmaceutical compositions and methods set forth herein.

3. Growth Inhibition Defined

“Inhibiting the growth” of a hyperproliferative lesion is broadly defined and includes, for example, a slowing or halting of the growth of the lesion. Inhibiting the growth of a lesion can also include a reduction in the size of a lesion or induction of apoptosis of the cells of the lesion. Induction of apoptosis refers to a situation wherein a drug, toxin, compound, composition or biological entity bestows apoptosis, or programmed cell death, onto a cell. In a specific embodiment, the cell is a tumor cell. In another embodiment the tumor cell is a head and neck cancer cell, a squamous cell carcinoma, a cervical cancer cell, or a cell of an anogenital wart. In further embodiments, the cell is a keratinocyte, an epithelial cell, a skin cell, a mucosal cell, or any other cell that can undergo transformation by a papillomavirus. Growth of a lesion can be inhibited by induction of an immune response against the cells of the lesion.

### F. PHARMACEUTICAL COMPOSITIONS

1. Definitions

The phrase “pharmaceutical composition” and “formulated” refer to molecular entities and compositions that do not produce an adverse, allergic or other untoward reaction when administered to a mammal or human, as appropriate. As used herein, a “pharmaceutical composition” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients. For instance, the composition for use as a toothpaste may include a flavorant or the composition may contain supplementary ingredients to make the formulation timed-release. Formulations are discussed in greater detail in the following sections.

Some of the pharmaceutical composition of the present invention are formulated for oral delivery. Oral delivery includes administration via the mouth of an animal or other mammal, as appropriate. Oral delivery also includes topical administration to any part of the oral cavity, such as to the gums, teeth, oral mucosa, or to a lesion in the mouth, such as a pre-neoplastic or neoplastic lesion. Oral delivery also includes delivery to a mouth wound or a tumor bed in the mouth.

In the context of the present invention, “topical administration” is defined to include administration to a surface of the body such as the skin, oral mucosa, gastrointestinal mucosa, eye, anus, cervix or vagina, or administration to the surface of the bed of an excised lesion in any of these areas (i.e., the surgical bed of an excised pharyngeal HNSCC or an excised cervical carcinoma), or administration to the surface of a hollow viscus, such as the bladder.

In still other embodiments of the present invention, the pharmaceutical composition is an enteric formulation. An enteric formulation is defined to include a pill, a capsule with a protective coating, or a suspension designed to withstand the low pH of the stomach. Such an enteric formulation would allow the delivery of the therapeutic or diagnostic genes to the small or large intestine.

2. Solid or Semi-Solid Formulations

The pharmaceutical compositions of the present invention may be formulated as a solid or semi-solid. Solid and semi-solid formulations refer to any formulation other than aqueous formulations. One of ordinary skill in the art would be familiar with formulation of agents as a solid or semi-solid.

Examples include a gel, a matrix, a foam, a cream, an ointment, a lozenge, a lollipop, a gum, a powder, a gel strip, a film, a hydrogel, a dissolving strip, a paste, a toothpaste, or a solid stick. Some of these formulations are discussed in greater detail as follows.

a. Gel

A gel is defined herein as an apparently solid, jelly-like material formed from a colloidal solution. A colloidal solution is a solution in which finely divided particles which are dispersed within a continuous medium in a manner that prevents them from being filtered easily or settled rapidly.

Methods pertaining to the formulation of gels are set forth in U.S. Pat. No. 6,828,308, U.S. Pat. No. 6,280,752, U.S. Pat. No. 6,258,830, U.S. Pat. No. 5,914,334, U.S. Pat. No. 5,888,493, and U.S. Pat. No. 5,571,314, each of which is herein specifically incorporated by reference in its entirety.

i. Topical Gel

Some of the pharmaceutical compositions set forth herein are formulated as a topical gel. For example, a nucleic acid expression construct may be formulated as a hydrophobic gel based pharmaceutical formulation. A hydrophobic gel may be formulated, for example, by mixing a pentamer cyclomethacone component (Dow Corning 245 fluid™) with a liquid suspension of a nucleic acid expression construct, hydrogenated castor oil, octyl palmitate and a mixture of cyclomethicone and dimethiconol in an 8:2 ratio. Preferably, the pentamer cyclomethicone component is approximately 40% of the gel, the liquid nucleic acid expression construct component is approximately 30.0% of the gel, the hydrogenated castor oil component is approximately 10% of the gel, the octyl palmitate component is approximately 10.0% of the gel and the cyclometnicone/dimethiconol component is approximately 10.0% of the gel. Each component listed above may be mixed together while heated at approximately 80-90° C. under vacuum. Upon lowering the temperature to, for example, 37° C., the nucleic acid expression construct component may then be added and the final gel composition should be allowed to cool to an ambient temperature. The final concentration of the nucleic acid expression construct in the hydrophobic gel formulation will depend on the type of construct employed and the administrative goal.

ii. Oral Gel Formulation

An oral gel pharmaceutical formulation for delivery of a nucleic acid expression construct may also be prepared using any method known to those of ordinary skill in the art. Such a pharmaceutical formulation may be applied to the oral cavity. Such a gel may be created, for example, by mixing water, potassium sorbate, sodium benzoate, disodium EDTA, hyaluronic acid and maltodextrin. After dissolution of the aforementioned ingredients, polyvinylpyrrolidone may be added added under stirring and vacuum, for example 30 mm Hg until complete solvation. Other ingredients, such as hydroxyethylcellulose and sweetners such as sodium saccharin may be stirred into the mixture while still under vacuum until complete salvation. Next, hydrogenated castor oil, benzalkonium chloride, and a mixture of propylene glycol and glycyrrhetinic acid may be stirred into the mixture, under the same conditions and in the order listed, until complete dissolution of the components. The mixture may form a gel by being stirred under vacuum for an additional 30 minutes. Table 4 provides a list of the aforementioned components in preferable concentrations.

Alternatively, a commercially available oral gel formulation comprising the aforementioned components, such as Gelclair® (Helsinn Healthcare, Switzerland), may be employed.

TABLE 4

| Component | % by weight |
|---|---|
| Sodium hyaluronate | 0.1 |
| Glycyrrhetinic acid | 0.06 |
| Polyvinylpyrrolidone | 9.0 |
| Maltodextrin | 6.00 |
| Propylene glycol | 2.94 |
| Potassium sorbate | 0.3 |
| Hydroxyethyl cellulose | 1.5 |
| Hydrogenated castor oil PEG-40 | 0.27 |
| Disodium EDTA | 0.1 |
| Benzalkonium chloride | 0.5 |
| Sodium saccharin | 0.1 |
| Depurated water | 78.60 |

The gel may subsequently be combined with one or more nucleic acid expression constructs according to the present invention. For example, 15 ml of the aforementioned gel may be mixed with 30-50 ml of a liquid suspension of a nucleic acid expression construct. The concentration of the nucleic acid expression construct both in the liquid suspension and in the gel formulation will depend on the type of expression construct employed and the therapeutic use.

iii. Ophthalmic Gel Formulations

The gel may be formulated for ophthalmic delivery by any method known to those of ordinary skill in the art. For example, an ophthalmic gel may be prepared for topical delivery of a nucleic acid expression construct to a subject by preparing first solution and a second solution followed by combining each solution.

One example of a first solution comprises approximately 200 g of purified water, 906 g boric acid, 0.13 g sodium borate, 1.0 g edetate disodium, 0.1 g benzalkonium chloride, 4.0 g sodium chloride, and 0.26 g of a lyophilized or liquid suspension nucleic acid expression construct. The particular concentration of the nucleic acid expression construct in the first solution will be determined by the type of expression construct and the therapy and the therapeutic goal.

A second solution may comprise, for example 760 g of purified water and 35 g of hydroxypropyl methyl cellulose. The hydroxypropyl methyl cellulose may be dissolved in the purified water by heating the water to approximately 90° C. until uniform dispersion.

Upon mixing the second solution, the temperature may be lowered such that the first solution may be aseptically added without inactivation of the nucleic acid expression construct. This method is only exemplary.

b. Matrix

A matrix is defined herein as a surrounding substance within which something else is contained, such as a pharmaceutical ingredient. Methods pertaining to the formulation of a conducting silicone matrix is set forth in U.S. Pat. No. 6,119,036, which is herein specifically incorporated by reference in its entirety. Also referenced are methods pertaining to formulation of a collagen based matrix, as in Doukas et al., 2001., and Gu et al. 2004.

c. Foam

A foam is defined herein as is a composition that is formed by trapping many gas bubbles in a liquid. Methods pertaining to the formulation and administration of foams are set forth in U.S. Pat. No. 4,112,942, U.S. Pat. No. 5,652,194, U.S. Pat. No. 6,140,355, U.S. Pat. No. 6,258,374, and U.S. Pat. No. 6,558,043, each of which is herein specifically incorporated by reference in its entirety.

A typical foam pharmaceutical formulation may, for example, be constructed by introducing a gas into a gel or aqueous pharmaceutical composition such that bubbles of the gas are within the pharmaceutical composition.

One example of preparation of a foam pharmaceutical formulation involving the use of a pressurized gas is discussed as follows. In brief, a nucleic acid of the present invention (12% w/v) may be mixed with mineral oil by stirring for approximately 30 minutes under a light vacuum to generate a first mixture. A solution of cetyl stearyl alcohol (6% w/v) in mineral oil may be added to the first mixture under the same conditions, to form a final mixture. The final mixture may be subsequently stirred for an additional 10 minutes. The final mixture may then be placed into an appropriate canister and pressurized with a propellant gas. The canister may have a mechanism for dispensing the final mixture, such as, for example a polyethylene valve of the type commonly found in pressurized canisters. This method is only exemplary.

d. Cream and Lotion

A cream is defined herein as semi-solid emulsion, which is defined herein to refer to a composition that includes a mixture of one or more oils and water. Lotions and creams are considered to refer to the same type of formulation. Methods pertaining to the formulation of creams are set forth in U.S. Pat. No. 6,333,194, U.S. Pat. No. 6,620,451, U.S. Pat. No. 6,261,574, U.S. Pat. No. 5,874,094, and U.S. Pat. No. 4,372,944, each of which is herein specifically incorporated by reference in its entirety.

e. Ointment

An ointment is defined herein as a viscous semisolid preparation used topically on a variety of body surfaces. Methods pertaining to the formulation of ointments are set forth in U.S. Pat. No. 5,078,993, U.S. Pat. No. 4,868,168, and U.S. Pat. No. 4,526,899, each of which is herein specifically incorporated by reference in its entirety.

By way of example, an ointment pharmaceutical formulation may comprise approximately 23.75 w/v % isostearyl benzoate, 23.85 w/v % bis(2-ethylhexyl)malate, 10.00 w/v % cyclomethicone, 5.00 w/v % stearyl alcohol, 10.00 w/v % microporous cellulose, 15.00 w/v % ethylene/vinyl acetate copolymer, 0.1 w/v % butylparaben, 0.1 w/v % propylparaben and 2.20 w/v % of the nucleic acid expression construct. The particular concentration of the nucleic acid expression construct in the first solution will be determined by the type of expression construct and the therapy and the administrative goal.

f. Powder

A powder is defined herein as fine particles to which any dry substance is reduced by pounding, grinding, or triturating.

g. Gel Strip

A gel strip is defined herein as a thin layer of gel with elastic properties. The gel may or may not be formulated with an adhesive. The gel may be formulated to slowly dissolve over time. For example, a gel designed for oral application may be designed to dissolve following application.

Another oral delivery system suitable for use in accordance with the present invention is a dissolvable strip. An example of such a device is the Cool Mint Listerine PocketPaks® Strips, a micro-thin starch-based film impregnated with ingredients found in Listerine® Antiseptic (Thymol, Eucalyptol, Methyl Salicylate, Menthol). Non-active strip ingredients include pullulan, flavors, aspartame, potassium acesulfame, copper gluconate, polysorbate 80, carrageenan, glyceryl oleate, locust bean gum, propylene glycol and xanthan gum.

h. Film

A film is defined herein as a thin sheet or strip of flexible material, such as a cellulose derivative or a thermoplastic resin, coated with a selected pharmaceutical ingredient. A lollipop is a lozenge attached to one end of a stick that is used as a handle.

A pharmaceutical film, lozenge, or lollipop of the present invention may be composed of ingredients, which may include, for example, xanthan gum, locust bean gum, carrageenan and pullulan. The ingredients may be hydrated in purified water and then stored overnight at 4° C., after which, coloring agents, copper gluconate, sweetners, flavorants and polyoxyethylene sorbitol esters such as polysorbate 80 and Atmos 300™ (ICI Co.), and the nucleic acid expression construct may be added to the mixture.

A film preparation of the present invention may be made for example, by pouring the aforementioned mixture into a mold and cast as a film, which may then be dried drying and cut into a desired size, depending on desired dosage of the pharmaceutical composition. A film may also be formulated without the addition of sweetners or flavorants, for example, if the formulation is not contemplated for oral application.

i. Lozenge

Solid lozenges are well known in the drug delivery field. A lozenge is a small solid of a therapeutic agent and other agents such as binders and sweeteners, that is designed to slowly dissolve when placed in the mouth of a subject. A lozenge may contain other ingredients known in such dosage forms such as acidity regulators, opacifiers, stabilizing agents, buffering agents, flavorings, sweeteners, coloring agents and preservatives. For example, solid formulations may be prepared as lozenges by heating the lozenge base (e.g., a mixture of sugar and liquid glucose) under vacuum to remove excess water and the remaining components are then blended into the mixture. The resulting mixture is then drawn into a continuous cylindrical mass from which the individual lozenges are formed. The lozenges are then cooled, subjected to a visual check and packed into suitable packaging.

One form of suitable packaging is a blister pack of a water-impermeable plastics material (e.g., polyvinylchloride) closed by a metallic foil. The patient removes the lozenge by applying pressure to the blister to force the lozenge to rupture and pass through the metal foil seal. Lozenges will normally be sucked by the patient to release the drug. Masticable solid dose formulations may be made by the methods used to prepare chewable candy products or chewing gums. For example, a chewable solid dosage form may be prepared from an extruded mixture of sugar and glucose syrup to which the drug has been added with optional addition of whipping agents, humectants, lubricants, flavors and colorings. See Pharmaceutical Dosage Forms: Tablets, Vol. 1, 2^{nd} Ed., Lieberman et al. (Eds.), 1989.

j. Lollipop

In another embodiment, the nucleic acid may be delivered orally in the form of a “lollipop” or “sucker.” Generally, lollipops and suckers are defined by a solid matrices into which a drug has been dispersed. They are solid or semi-solid at room temperature, and are dissolved by contact with an aqueous environment, i.e., the mouth. Dissolution of the matrices (and hence, release of the drug) may be enhanced by the increased temperature (as compared to ambient or room temperature) of the mouth. Lollipops can be a convenient vehicle for administering a drug to a patient, and differ from a lozenge in that the lollipop can be temporarily removed from the patient's mouth. This enables the patient to communicate orally when necessary, and to control the duration and extent of delivery.

A lollipop (or film or lozenge) of the present invention may be composed of ingredients, which may include, for example, xanthan gum, locust bean gum, carrageenan and pullulan. The ingredients may, for example, be hydrated in purified water and then stored overnight at 4° C., after which, coloring agents, copper gluconate, sweetners, flavorants and polyoxyethylene sorbitol esters such as polysorbate 80 and Atmos 300™ (ICI Co.), and the nucleic acid expression construct may be added to the mixture.

A lollipop or lozenge preparation of the present invention may be made for example, by pouring the aforementioned mixture into a mold of desired size, which may then be dried. Prior to drying, a typical lollipop holding stick would be inserted into the mold for a lollipop preparation.

k. Hydrogel

A hydrogel is defined herein as a network of polymer chains that are sometimes found as a colloidal gel in which water is the dispersion medium. Using the teachings of the specification and the knowledge of those skilled in the art, one can also compose a pharmaceutical formulation as hydrogel such that it may be complexed with a nucleic acid expression construct for topical delivery to a subject. An example of a hydrogel formulation for the delivery of nucleic acids in a viral vector is shown below.

For instance, bovine type I collagen (available, e.g., from Collagen Corporation, Fremont, Calif.), sodium alginate and a liquid suspension of a virus vector may be mixed together to form a hydrogel precursor. The proportion of collagen:alginate, on a dry weigh basis, may be from about 7:3 to about 4:6. After forming the hydrogel precursor mixture, a hydrogel matrix is formed therefrom by solidifying the mixture. The mixture can be solidified to create a hydrogel by contacting it with polyvalent cations such as Ca²⁺. Preferably the Preferably, the Ca²⁺ solution should be at least 2.5 millimolar. The concentration of the nucleic acid expression construct will depend on the type of construct used and the administrative goal.

l. Dissolving Strip

A dissolving strip is defined herein as a film contemplated to dissolve in the presence of an aqueous environment such as a body cavity.

m. Paste and Toothpaste

A paste is defined herein as a substance that behaves as a solid until a sufficiently large load or stress is applied, at which point it flows like a fluid. A toothpaste is defined herein as a paste or gel used to clean and improve the aesthetic appearance of teeth. A paste dentifrice that may include water, binders, abrasives, flavoring agents, foaming agents, and humectants. Methods pertaining to the formulation of toothpastes are set forth in U.S. Pat. No. 4,627,979, U.S. Pat. No. 6,508,647, U.S. Patent Appn. 20020045148, and U.S. Patent Appn. 20040018155, each of which is herein specifically incorporated by reference in its entirety.

Using the teachings of the specification and the knowledge of those skilled in the art, one may elect to construct a toothpaste pharmaceutical formulation for delivery of a nucleic acid expression construct to the oral cavity of a subject. A toothpaste according to the present invention, for example, may have the following formulation: 1% by weight of a polishing material such as silica or calcium carbonate 20-75% by weight of a polyol such as glycerol or polyethylene glycol, 20-55% by weight of sodium bicarbonate, 0.001-40% by weight of sodium lauryl sulfate, 0.001-20% by weight titanium dioxide, 0.1-10% by weight of a thickener such as guar gum or pectin, 0.001-5% by weight of sodium saccharin and 10-30% by weight of the nucleic acid expression construct in a liquid formulation. The particular concentration of the nucleic acid expression construct in the first solution will be determined by the type of expression construct and the therapy and the therapeutic goal.

n. Suppositories and Pessaries

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%. A method pertaining to pharmaceutical formulations of suppositories is set forth in U.S. Pat. No. 6,982,091, which is specifically incorporated by reference in its entirety.

A suppository formulation according to the present invention may be formulated, for example, by combining a selected nucleic acid, hydroxypropyl methylcellulose, a lipophilic carrier and a permeation enhancer. For instance, a suppository may be formulated by dissolving hydroxypropyl methylcellulose (e.g., METHOCEL K, HPMC K15M obtained from Dow Chemical, Midland, Mich. (8%/wt); and a permeation enhancing polyoxyethylene alkyl ether (e.g., TRANSCUTOL® obtained from Gattefossé (17%/wt)., into the lipophilic carrier SUPPOCIRE CS2 obtained from Gattefossé, Westwood, N.J. (75% wt). The selected nucleic acid may be stirred into the mixture and poured into an appropriate suppository mold and allowed to solidify prior to topical application.

o. Gum

The present invention also contemplates gum-based pharmaceutical formulation of the present invention may be constructed for oral delivery of a nucleic acid to a subject.

By way of example, gum base pellets may be frozen to increase hardness and mechanically ground into a powder form. Subsequently, the gum powder may be elevated to room temperature and mixed with a sweetener, such as fructose or aspartame, comprising approximately 20-65% by weight of the gum-sweetener composition. The gum-sweetener composition may then be supplemented with a liquid suspension of a nucleic acid of the present invention. For instance, the amount of the liquid suspension of the nucleic acid may be approximately equal to 2% by weight of the gum-sweetener composition. The mixture of the gum-sweetener composition and the nucleic acid may then be pressed into a desired shape and administered to a subject. Other methods of formulating a therapeutic agent in a gum are contemplated by the present invention, and are well-known to those of ordinary skill in the art.

3. Diluents and Carriers

In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like.

Solid forms suitable for solution in, or suspension in, liquid prior to topical use are also contemplated by the present invention.

The solid and semisolid formulations of the present invention may contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; a fragrance, and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. Preservatives, dyes, and flavorings known to those of ordinary skill in the art are contemplated.

The solid and semisolid formulations of the present invention contemplated for use on skin surfaces may include other ingredients, which are commonly blended in compositions for cosmetic purposes. For example, such cosmetic ingredients include: waxes, oils, humectants, preservatives, antioxidants, ultraviolet absorbers, ultraviolet scattering agents, polymers, surface active agents, colorants, pigments, powders, drugs, alcohols, solvents, fragrances, flavors, etc, are contemplated. Specific examples of cosmetic compositions include, but are not limited to: make-up cosmetics such as lipstick, lip-gloss, lip balm, skin blemish concealer, and lotion. Methods pertaining to cosmetic formulations designed for use as pharmaceutical carriers are set forth in U.S. Pat. No. 6,967,023, U.S. Pat. No. 6,942,878, U.S. Pat. No. 6,881,776, U.S. Pat. No. 6,939,859 and U.S. Pat. No. 6,673,863, each of which is herein specifically incorporated by reference in its entirety.

4. Aqueous Formulations

Certain of the pharmaceutical compositions of the present invention can be formulated as aqueous compositions. Aqueous compositions of the present invention comprise an effective amount of the nucleic acid, dissolved or dispersed in a pharmaceutically acceptable carrier or aqueous medium.

Administration of certain embodiments of the pharmaceutical compositions set forth herein will be via any common route so long as the target tissue is available via that route. For example, this includes esophageal, gastric, oral, nasal, buccal, anal, rectal, vaginal, topical ophthalmic, or applications to skin. Such compositions would normally be administered as pharmaceutically acceptable compositions that include physiologically acceptable carriers, buffers or other excipients. Examples of other excipients include fragrances and flavorants.

The formulation may be in a liquid form or suspension. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Examples of aqueous compositions for oral administration include a mouthwash, mouthrinse, a coating for application to the mouth via an applicator, or mouthspray. Mouthwash formulations are well-known to those of skill in the art. Formulations pertaining to mouthwashes and oral rinses are discussed in detail, for example, in U.S. Pat. No. 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, U.S. Pat. No. 4,919,918, U.S. Patent Appn. 20040076590, U.S. Patent Appn. 20030152530, and U.S. Patent Appn. 20020044910, each of which is herein specifically incorporated by reference into this section of the specification and all other sections of the specification.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions such as mouthwashes and mouthrinses. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

For oral administration the expression cassette of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For oral administration the expression cassette of the present invention may also be incorporated with dyes to aid in the detection of hyperproliferative lesions such as toluidene blue O dye and used in the form of non-digestible mouthwashes, oral rinses and dentrifrices. A mouthwash may be prepared incorporating the active ingredient in the required amount in an orally administered dye composition, such as a composition of toluidene blue O dye, a buffer, a flavorant, a preservative, acetic acid, ethyl alcohol and water. Methods and formulations pertaining to the use of Toluidene Blue O dye in the staining of precancerous and cancerous lesions may be found in, for example, U.S. Pat. No. 4,321,251, U.S. Pat. No. 5,372,801, U.S. Pat. No. 6,086,852, and U.S. Patent Appn. 20040146919, each of which is specifically incorporated by reference in its entirety.

Examples of aqueous compositions for application to topical surfaces include emulsions or pharmaceutically acceptable carriers such as solutions of the active compounds as free base or pharmacologically acceptable salts, active compounds mixed with water and a surfactant, and emulsions. Emulsions are typically heterogenous systems of one liquid dispersed in another in the form of droplets usually exceeding 0.1 um in diameter. (Idson, 1988; Rosoff, 1988; Block, 1988; Higuchi et al., 1985). Emulsions are often biphasic systems comprising of two immiscible liquid phases intimately mixed and dispersed with each other. In general, emulsions may be either water in oil (w/o) or of the oil in water (o/w) variety. Methods pertaining to emulsions that may be used with the methods and compositions of the present invention set forth in U.S. Pat. No. 6,841,539 and U.S. Pat. No. 5,830,499, each of which is herein specifically incorporated by reference in its entirety. Aqueous compositions for application to the skin may also include dispersions in glycerol, liquid polyethylene glycols and mixtures thereof. Under ordinary conditions of storage and use, these preparations contain a preservative to prevent the growth of microorganisms.

The use of liposomes and/or nanoparticles is also contemplated in the present invention. The formation and use of liposomes is generally known to those of skill in the art, and is also described below. Liposomes are also discussed elsewhere in this specification.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made. Methods pertaining to the use of nanoparticles that may be used with the methods and compositions of the present invention include U.S. Pat. No. 6,555,376, U.S. Pat. No. 6,797,704, U.S. Patent Appn. 20050143336, U.S. Patent Appn. 20050196343 and U.S. Patent Appn. 20050260276, each of which is herein specifically incorporated by reference in its entirety.

Examples of aqueous compositions contemplated for esophageal or stomach delivery include liquid antacids and liquid alginate-raft forming compositions. Liquid antacids and liquid sucralfate or alginate-raft forming compositions are well known to those skilled in the art. Alginates are pharmaceutical excipients generally regarded as safe and used therefore to prepare a variety of pharmaceutical systems well documented in the patent literature, for example, in U.S. U.S. Pat. No. 6,348,502, U.S. Pat. No. 6,166,084, U.S. Pat. No. 6,166,043, U.S. Pat. No. 6,166,004, U.S. Pat. No. 6,165,615 and U.S. Pat. No. 5,681,827, each of which is herein specifically incorporated by reference into this section of the specification and all other sections of the specification.

Oral formulations contemplated for esophageal or stomach delivery include such normally employed excipients as, for example, pharmaceutical grades of hydroxylethyl cellulose, water, simethicone, sodium carbonate, sodium saccharin, sorbital and/or the like. Flavorants may also be employed. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

One may also use solutions and/or sprays, hyposprays, aerosols and/or inhalants in the present invention for administration. One example is a spray for administration to the aerodigestive tract. The sprays are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Methods pertaining to spay administration are set forth in U.S. Pat. No. 6,610,272 U.S. Pat. No. 6,551,578 U.S. Pat. No. 6,503,481, U.S. Pat. No. 5,250,298 and U.S. Pat. No. 5,158,761, each of which is specifically incorporated by reference into this section of the specification and all other sections of the specification.

Administration of certain embodiments of the aqueous pharmaceutical compositions set forth herein will be via any common route so long as the target tissue is available via that route. For example, this includes oral, nasal, buccal, anal, rectal, vaginal, or topical ophthalmic. Such compositions would normally be administered as pharmaceutically acceptable compositions that include physiologically acceptable carriers, buffers or other excipients. Examples of other excipients include fragrances and flavorants.

a. Mouthwash Formulations

Using the teachings of the specification and the knowledge of those skilled in the art, one can compose a pharmaceutical formulation for delivery of a nucleic acid expression construct as a mouthwash for application to the oral cavity. For instance, the mouthwash formulation may comprise a typical mouthwash solution and a suspension of the selected nucleic acid expression construct. One such formulation of a typical mouthwash solution which may be employed according to the present invention is shown in table 5.

TABLE 5

| Ingredient | Weight | |
|---|---|---|
| Calcium Chloride | 1.39 | g |
| dehydrate | | |
| Sodium Chloride | 11.42 | g |
| Sodium Benzoate | 0.050 | g |
| Disodium Phosphate | 0.634 | g |
| Monosodium | 0.200 | g |
| phosphate Monohydrate | | |
| Flavoring | 0.1 | ml |
| Distilled Water | q.s to 2000 | ml |

The mouthwash formulation may be mixed with the nucleic acid expression construct, for example, an adenoviral vector. The concentration of the nucleic acid expression construct would depend on the particular construct employed and the therapeutic goal. The formulation may be subsequently applied to the oral cavity of a subject. For instance, the application may be via a swab, by gargling or by swishing. The application may be repeated once or several times.

Alternatively, using the teachings of the specification and the knowledge of those skilled in the art, one can compose a mouthwash pharmaceutical formulation incorporating a precancerous and cancerous lesion detecting dye for delivery of a nucleic acid expression construct to the oral cavity. For instance, the nucleic acid construct may be mixed with a dye containing mouthwash. One such method and formulation involving a mouthwash containing a dye capable of detecting precancerous and cancerous lesions in the oral cavity is shown below.

Toluidene blue 0 dye (1% w/v), a flavorant (0.2% w/v) and sodium acetate trihydrate buffering solution may be, for instance, dissolved in a solution of water, glacial acetic acid, and ethanol, to form a dye containing mouthwash solution. A nucleic acid according to the present invention may be subsequently added to the mouthwash solution in an appropriate amount. The concentration of the nucleic acid in the mouthwash will depend on the type of nucleic acid construct employed and the administrative goal.

By way of example, the pharmaceutical formulation may be administered to a subject using the following steps: 1) the subject gargles and swishes approximately 15 ml of a rinse solution comprising 1% acetic acid and sodium benzoate preservative in water for 20 seconds followed by expectoration, 2) the subject gargles and swishes approximately 15 ml of water for 20 seconds followed by expectoration, 3) the subject gargles and swishes approximately 30 ml of the pharmaceutical formulation for 60 seconds followed by expectoration, 4) step 1 is repeated twice, and 5) step 2 is repeated twice. Other methods of administering these compositions are contemplated, and are well-known to those of ordinary skill in the art.

Observations of the oral cavity may be conducted under appropriate magnification and appropriate light immediately after application of the pharmaceutical formulation to examine the oral cavity for the presence of dyed precancerous and cancerous cells. Subsequent observations of the oral cavity may be conducted after a period of time to allow for transduction of the cells of the oral cavity with a nucleic acid of the present invention. Such observations may be conducted under appropriate magnification and appropriate light.

b. Douche and Enema Formulation

The nucleic acids may further be formulated as a douche or enema. For example, the chosen nucleic acid expression construct may be mixed with a typical douche or enema composition well-known to those of ordinary skill in the art. The formulation of a typical douche or enema is shown in table 6.

TABLE 6

| Ingredient | Weight | |
|---|---|---|
| Carboxymethyl | 500 | g |
| cellulose | | |
| Sorbitol | 5 | g |
| Distilled water | 60 | ml |

According to the teachings of the specification and the knowledge of those skilled in the art, a typical douche or enema formulation, for instance the formulation shown in table 6, may be mixed with the chosen nucleic acid construct. The concentration of the nucleic acid expression construct in a douche or enema formulation would depend on the type of expression construct employed and administrative goal. The formulation may subsequently be administered anally, vaginally, or via catheter to the subject.

5. Non-Ionic Surfactant Formulations

The pharmaceutical formulation may be a non-ionic surfactant for topical delivery. Such a formulation may be comprised of, for example, three separate components. The first component can be non-ionic lamellar layer forming surfactant. The second component can be another surfactant. The final component may be a nucleic acid expression construct, such as an adenoviral vector. The nucleic acid expression construct may be either lyophilized or suspended, for example, in distilled phosphate buffered saline and 10% glycerol at pH 7.4. Examples of lamellar layer forming surfactants that may be used are found in table 7.

TABLE 7

| Tradename | Chemical Name | HLB | Supplier |
|---|---|---|---|
| L-595 | sucrose laurate | 5 | Ryoto |
| | ester | | |
| | (30% mono 70% | | |
| | di/tri/poly) | | |
| Tween ® 81 | POE(5) Sorbitan | 10.0 | ICI |
| | monooleate | | |
| Tween ® 85 | POE(2) Sorbitan | 11.0 | ICI |
| | trioleate | | |
| Span ® 20 | Sorbitan | 8.6 | Sigma |
| | monolaurate | | |
| Span ® 80 | Sorbitan | 4.3 | ICI |
| | monooleate | | |
| Span ® 85 | Sorbitan trioleate | 1.8 | Sigma |
| Serdox ® | POE(4.5)Oleyester | 8.7 | Servo, |
| NOG | | | Delden |
| C12EO3 | POE(3) | 904 | Servo, |
| | Dodecylether | | Deldan |

Examples of a second surfactant are found in table 8.

TABLE 8

| | Chemical | | |
|---|---|---|---|
| Tradename | Name | HLB | Supplier |
| C12EO7 | POE(7) | 12.9 | Servo, |
| | dodecylether | | Delden |
| Brij ® 96 | POE(10) | 12.4 | ICI |
| | oleylether | | |
| L-1695 | Sucrose | 16 | Ryoto |
| | laurate | | |
| Peg-8- | POE(8) | 13.5 | Diopeg |
| laurate | dodecylester | | |
| Serdox ® | POE(10) | 12.4 | Servo, |
| NOG S-440 | oleylester | | Delden |
| Serdox | Sorbitan | 1.8 | Sigma |
| NOG ® S-440 | trioleate | | |

The formulation for a non-ionic surfactant for adenoviral vector topical delivery may, for example, be formulated by mixing sucrose laurate ester (L-595) and POE(7) dodecyl ether (C12EO7) in an amount required to obtain a final aqueous dispersion containing 5 wt %. The mixture may, for example, be a mixture in a ratio of 0.3:0.7 or 0.2:0.8 or 0.1:0.9 of the first and second surfactant respectively. These surfactants may be may first be dissolved, for example, in a 3 to 1 solution of chloroform to methanol after which, the solvents can be evaporated. The remaining dry film may then be hydrated by adding a liquid suspension of the nucleic acid expression construct, for example approximately, 5 ml of such a suspension.

6. Antacid Formulations

In some embodiments of the present invention, the pharmaceutical compositions further include one or more antacids. Any method of formulation with an antacid is contemplated by the present invention. In preparing an antacid formulation according to the teachings of the specification and the knowledge of those skilled in the art, one may first wish to suspend the nucleic acid expression construct in a liquid formulation. For example, an adenoviral vector may be suspended in an aqueous formulation of distilled phosphate buffered saline and 10% glycerol at pH 7.4. The amount of an adenoviral vector or any nucleic acid expression construct will depend on the therapeutic goal. An additional component of such a liquid formulation may be an antacid, which would allow the pH of the gastric mucosa to be temporarily raised upon administration to a subject. The antacid, for example, may include ingredients such as aluminum hydroxide or magnesium hydroxide. Additionally, other ingredients often found in commercially available liquid antacid formulations may be added to such a pharmaceutical formulation. Such ingredients often include, but are not limited to: butylparaben, hydroxypropyl methylcellulose, microcrystalline cellulose, propylparaben, sodium carboxymethylcellulose, sodium saccharin, sorbitol, distilled water, and flavorants.

7. Alginate Raft Formulations

Alginate raft formulations are also contemplated by the present invention. An alginate raft is defined herein to refer to as a gel entrapped with gas that is formed by the precipitation of alginic acid in the presence of gastric acid. For example, the nucleic acid expression construct may be comprised in an adenoviral vector.

In preparing an alginate raft formulation according to the teachings of the specification and the knowledge of those skilled in the art, the nucleic acid expression construct, for example, may be suspended in an alginate raft forming liquid composition. An example of such a nucleic acid expression construct contemplated in an alginate raft forming pharmaceutical composition may be, for example, an adenoviral vector. The adenoviral vector could be mixed with an alginate raft forming liquid. Such an alginate raft forming liquid may comprise ingredients found in commercially available formulations of this type, such as aluminum hydroxide, magnesium carbonate, sodium bicarbonate and alginic acid. The commercially available alginic raft formulation Gaviscon® (Glaxo Smith Kline) is a preferred example. In the presence of gastric acid, alginates precipitate, forming a gel. Alginate raft forming compositions may also contain sodium or potassium bicarbonate; in the presence of gastric acid, the bicarbonate is converted to carbon dioxide, which is entrapped within the gel precipitate, thus converting it into a foam that ‘floats’ on the surface of the gastric contents. Raft formation occurs within a few seconds of dosing, and the raft can be retained in stomach for several hours.

An alginate raft forming composition, for example, may be formulated by mixing sodium alginate (500 mg), sodium bicarbonate (250 mg), calcium carbonate (150 mg), methyl paraben (40 mg), propyl paraben (6 mg) and a crosslinked polyacrylic acid such as Carbopol® (Noveon). The ingredients may be mixed together and dissolved in the aqueous formulation containing the adenoviral vector to a final volume of 10 ml. The alginate raft pharmaceutical formulation of the present invention may subsequently swallowed by a subject. Other examples of alginate raft forming formulations may be found in U.S. Pat. No. 6,348,502, U.S. Pat. No. 5,681,827 and U.S. Pat. No. 5,456,918, each of which is herein specifically incorporated by reference into this section of the specification and all other sections of the specification.

8. Compositions Using Viral Vectors

Where clinical application of a viral expression vector according to the present invention is contemplated, it will be necessary to prepare the complex as a pharmaceutical composition appropriate for the intended application. Generally, this will entail preparing a pharmaceutical composition that is essentially free of pyrogens, as well as any other impurities that could be harmful to humans and other mammals. One also will generally desire to employ appropriate salts and buffers to render the complex stable and allow for complex uptake by target cells.

9. Emulsion Formulations

Using the teachings of the specification and the knowledge of those skilled in the art, one can also compose a pharmaceutical formulation as an emulsion for topical delivery of a nucleic acid expression construct. For instance, the nucleic acid expression construct may be a viral vector, such as an adenoviral vector. One example of an emulsion formulation for the delivery of nucleic acids in a viral vector is as follows:

Poly(lactic-glycolic) acid (PLGA) may be dissolved in dichloromethane and mixed with an aqueous suspension of a viral vector. For instance, 1 ml of dichloromethane and 0.05 ml of an aqueous suspension of virus may be used. The solution may then be vortexed for approximately 30 seconds to form a water in oil emulsion. 1 ml of 1% poly vinyl alcohol may then be added to the emulsion and subsequently vortexed for an additional 30 seconds. After the second round of vortexing, the emulsion may then be added to 100 ml of a 0.1% poly vinyl alcohol solution and stirred for an additional 30 minutes. Next, the dichloromethane may be removed by applying a vacuum to the emulsion while stirring for 2.5 hours. After removal of the dichloromethane, the emulsion may then be filtered with 0.2 μm nylon filters and washed with 500 ml of phosphate buffered saline. In the case of emulsions containing viruses, a protective agent may be employed to prevent the denaturation of the viral proteins. Typical protective agents may include, for example, glycerol, sucrose and bovine serum albumin.

10. Nanoparticle Liposome Formulation

The present invention also includes nanoparticle liposome formulations for topical delivery of a nucleic acid expression construct. For instance, the liposome formulation may comprise DOTAP and cholesterol. An example of such a formulation containing a nucleic acid expression construct is shown below.

Cationic lipid (DOTAP) may be mixed with the neutral lipid cholesterol (Chol) at equimolar concentrations (Avanti Lipids). The mixed powdered lipids can be dissolved in HPLC-grade chloroform (Mallinckrodt, Chesterfield, Mo.) in a 1-L round-bottomed flask. After dissolution, the solution may be rotated on a Buchi rotary evaporator at 30° C. for 30 min to make a thin film. The flask containing the thin lipid film may then be dried under a vacuum for 15 min. Once drying is complete, the film may be hydrated in 5% dextrose in water (D5W) to give a final concentration of 20 mM DOTAP and 20 mM cholesterol, referred to as 20 mM DOTAP:Chol. The hydrated lipid film may be rotated in a water bath at 50° C. for 45 min and then at 35° C. for 10 min. The mixture may then be allowed to stand in the parafilm-covered flask at room temperature overnight, followed by sonication at low frequency (Lab-Line, TranSonic 820/H) for 5 min at 50° C. After sonication, the mixture may be transferred to a tube and heated for 10 min at 50° C., followed by sequential extrusion through Whatman (Kent, UK) filters of decreasing size: 1.0, 0.45, 0.2 and 0.1 μm using syringes. Whatman Anotop filters, 0.2 μm and 0.1 μm, may be used. Upon extrustion, the liposomes can be stored under argon gas at 4° C.

A nucleic acid expression construct in the form of plasmid DNA, for example 150 μg may be diluted in D5W. Stored liposomes may also be diluted in a separate solution of D5W. Equal volumes of both the DNA solution and the liposome solution can then be mixed to give a final concentration of, for example, 150 μg DNA/300 μl volume (2.5 μg/5 μl). Dilution and mixing may be performed at room temperature. The DNA solution mau then be added rapidly at the surface of the liposome solution by using a Pipetman pipet tip. The DNA:liposome mixture can then be mixed rapidly up and down twice in the pipette tip to form DOTAP:Cholesterol nucleic acid expression construct complexes.

Using the teachings of the specification and the knowledge of those skilled in the art, one can conduct tests to determine the particle size of the DOTAP:Chol-nucleic acid expression complex. For instance, the particle size of the DOTAP:Chol-nucleic acid expression construct complex may be determined using the N4-Coulter Particle Size analyzer (Beckman-Coulter). For this determination, 5 μl of the freshly prepared complex should be diluted in 1 ml of water prior to particle size determination. Additionally, a spectrophotometric reading of the complex at O.D. 400 nm may also be employed in analysis. For this analysis, 5 μl of the sample may be diluted in 95 μl of D5W to make a final volume of 100 μl. Applying the formulation techniques above with the size analysis methods should demonstrate a size of the complex between 374-400 nm.

11. Popsicle Formulation

Using the teachings of the specification and the knowledge of those skilled in the art, one can compose a pharmaceutical formulation for delivery of a nucleic acid expression construct as a popsicle for application to the oral cavity or gastrointestinal tract. A popsicle is defined herein as a frozen liquid formulation comprising a hand held applicator such as a stick or a sheath. For instance, the popsicle formulation may comprise a popsicle formulation and a suspension of the selected nucleic acid expression construct. Accordingly, a popsicle formulation may be composed of a frozen solution of a sugar (20% w/v), a flavorant (1.0% w/v), a colorant (0.5% w/v) and an aqueous solution containing a nucleic acid of the present invention (80% w/v). The components of the formulation may be mixed together in liquid form and subsequently frozen in a popsicle mold. Additional examples of popsicle formulations may be found for example in U.S. Pat. No. 5,194,269 and U.S. Pat. No. 5,660,866, each of which is herein specifically incorporated by reference in their entirety.

12. Transdermal or Transcutaneous Delivery Devices

Certain embodiments of the present invention pertain to transdermal or transcutaneous delivery devices for delivery of a therapeutic agent comprising a patch and a nucleic acid encoding an amino acid sequence capable of preventing or inhibiting a disease in a subject, such as the growth of a hyperproliferative lesion in a subject. The therapeutic agent is in contact with a surface of the patch. As set forth above, the therapeutic agent includes a nucleic acid sequence encoding an amino acid sequence capable of preventing or inhibiting disease in a subject, such as the growth of a hyperproliferative lesion.

The patch can be composed of any material known to those of ordinary skill in the art. Further, the patch can be designed for delivery of the therapeutic agent by application of the patch to a body surface of a subject, such as a skin surface, the surface of the oral mucosa, a wound surface, or the surface of a tumor bed. The patch can be designed to be of any shape or configuration, and can include, for example, a strip, a bandage, a tape, a dressing (such as a wound dressing), or a synthetic skin. Formulations pertaining to transdermal or transcutaneous patches are discussed in detail, for example, in U.S. Pat. No. 5,770,219 U.S. Pat. No. 6,348,450, U.S. Pat. No. 5,783,208, U.S. Pat. No. 6,280,766 and U.S. Pat. No. 6,555,131, each of which is herein specifically incorporated by reference into this section and all other sections of the specification.

In some embodiments, the device may be designed with a membrane to control the rate at which a liquid or semi-solid formulation of the therapeutic agent can pass through the skin and into the bloodstream. Components of the device may include, for example, the therapeutic agent dissolved or dispersed in a reservoir or inert polymer matrix; an outer backing film of paper, plastic, or foil; and a pressure-sensitive adhesive that anchors the patch to the skin. The adhesive may or may not be covered by a release liner, which needs to be peeled off before applying the patch to the skin. In some embodiments, the therapeutic agent is contained in a hydrogel matrix.

In some embodiments, it is desirable to transport the therapeutic agent(s) through the skin. Accordingly, topical patch formulations may include a skin permeability mechanism such as: a hydroxide-releasing agent and a lipophilic co-enhancer; a percutaneous sorbefacient for electroporation; a penetration enhancer and aqueous adjuvant; a skin permeation enhancer comprising monoglyceride and ethyl palmitate; stinging cells from cnidaria, dinoflagellata and myxozoa; and/or the like. Formulations pertaining to skin permeability mechanisms are discussed in detail, for example, in U.S. Pat. No. 6,835,392, U.S. Pat. No. 6,721,595, U.S. Pat. No. 6,946,144, U.S. Pat. No. 6,267,984 and U.S. Pat. No. 6,923,976, each of which is specifically incorporated by reference into this section of the specification and all other sections of the specification. Also contemplated is: microporation of skin through the use of tiny resistive elements to the skin followed by applying a patch containing adenoviral vectors as referenced by Bramson et al. (2003); a method of increasing permeability of skin through cryogen spray cooling as referenced by Tuqan et al. (2005); jet induced skin puncture as referenced by Baxter et al. (2005); heat treatment of the skin as referenced by Akomeah et al. (2004); and scraping of the skin to increase permeability.

In other embodiments, the patch is designed to use a low power electric current to transport the therapeutic agent through the skin. In other embodiments, the patch is designed for passive drug transport through the skin or mucosa. In other embodiments, the device is designed to utilize iontophoresis for delivery of the therapeutic agent.

The device may include a reservoir wherein the therapeutic agent is comprised in a solution or suspension between the backing layer and a membrane that controls the rate of delivery of the therapeutic agent. In other embodiments, the device includes a matrix comprising the therapeutic agent, wherein the therapeutic agent is in a solution or suspension dispersed within a collagen matrix, polymer, or cotton pad to allow for contact of the therapeutic agent with the skin. In some embodiments, an adhesive is applied to the outside edge of the delivery system to allow for adhesion to a surface of the subject.

In some embodiments, the device is composed of a substance that can dissolve on the surface of the subject following a period of time. For example, the device may be a file or skin that can be applied to the mucosal surface of the mouth, wherein the device dissolves in the mouth after a period of time. The therapeutic agent, in these embodiments, may be either applied to a single surface of the device (i.e., the surface in contact with the subject), or impregnated into the material that composes the device.

In some embodiments, the device is designed to incorporate more than one therapeutic agent. The device may comprise separate reservoirs for each therapeutic agent, or may contain multiple therapeutic agents in a single reservoir.

Further, the device may be designed to vary the rate of delivery of the therapeutic agent based on bodily changes in the subject, such as temperature or perspiration. For example, certain agents may be comprised in a membrane covering the therapeutic agent that respond to temperature changes and allow for varying levels of drug to pass through the membrane. In other embodiments, transdermal or transcutaneous delivery of the therapeutic agent can be varied by varying the temperature of the patch through incorporation of a temperature-control device into the device.

One of ordinary skill in the art would be familiar with methods and techniques for transdermal and transcutaneous delivery of drugs using patches.

Using the teachings of the specification and the knowledge of those skilled in the art, one may elect to topically deliver a nucleic acid expression construct using a transdermal delivery patch. In preparing a transdermal patch according to the teachings of the specification and the knowledge of those skilled in the art, a nucleic acid expression construct, an adhesive, and a permeation enhancer may be mixed together and dispensed onto a siliconized polyester release liner (Release Technologies, Inc., W. Chicago, Ill.). For example the transdermal patch formulation may consist of approximately 88% by composition of an acrylic copolymer adhesive, 2% of a nucleic acid expression construct, and 10% of a sorbitan monooleate permeation enhancer such as ARACEL 80™ (ICI Americas, Wilmington, Del.). The mixture may then be dried and stored for treatment of a subject.

13. Adhesives

In some embodiments, the pharmaceutical composition includes one or more adhesives. An adhesive is defined herein to generally refer to an agent or combination of agents that promotes or facilitates contact of the nucleic acid with a surface, or promotes or facilitates contact of one surface with another surface.

Adhesives for use in pharmaceutics and medicine are well-known to those of ordinary skill in the art, and include topical skin adhesives such as sterile, liquid glue, as well as solid or semi-solid adhesives. Adhesives for use in the present invention also include adhesives that are liquid upon application, but which rapidly dry to a solid consistency.

Exemplary adhesives for use in the compositions and methods of the present invention include acrylates, such as cyanoacrylate, methacrylates, and alkyl acrylates. Other exemplary adhesives include hydrocolloids, hydrogels, polyisobutylene, and adhesives that are based on a gel matrix, such as polyacrylic acid-based gel matrix adhesives.

Tissue adhesives are also contemplated for use in the pharmaceutical compositions and methods of the present invention. Compositions pertaining to tissue adhesives are discussed in detail in U.S. Patent Appn. 20040199207, U.S. Patent Appn. 20030119985, U.S. Patent Appn. 20020116026, U.S. Patent Appn. 20020037323, U.S. Pat. No. 6,723,114, U.S. Pat. No. 6,596,318, U.S. Pat. No. 6,329,337, U.S. Pat. No. 6,310,036, U.S. Pat. No. 6,299,631, and U.S. Pat. No. 6,251,370, each of which is herein specifically incorporated by reference.

Using the teachings of the specification and the knowledge of those skilled in the art, one can topically deliver a nucleic acid expression construct with an adhesive pharmaceutical formulation. For instance, an adhesive pharmaceutical formulation can be constructed by mixing a cyanoacrylate based adhesive, such as methoxy propyl cyanoacrylate with a copolymer. For example, the copolymer may be a ε-caprolactone-glycolide/lactide-glycolide copolymer.

A ε-caprolactone-glycolide/lactide-glycolide copolymer may be constructed by mixing, for example, 0.13 moles of glycolide with 1.18 moles of ε-caprolactone and a catalytic amount of stannous octoate (0.262 mmole) and 1-decanol (3.275 mmole). The mixture may be heated to a temperature of 170° C. and stirred for approximately 30 minutes, followed by cooling the mixture to 120° C. to allow the addition of approximately 0.65 moles of glycolide and 0.52 moles of dl-lactide. The mixture may then be re-heated to a temperature of 170° C. and stirred for an additional 6.5 hours. Any unreacted monomer may then be removed from the copolymer solution by stirring the mixture at a temperature of, for example 130° C. under reduced pressure for 1.5 hours.

The pharmaceutical formulation of methoxy propyl cyanoacrylate, copolymer and nucleic acid expression construct could be mixed together and applied to a topical surface of a subject. For instance, the mixture could be approximately 90% methoxy propyl cyanoacrylate, 5% copolymer and 5% of the nucleic acid expression construct. Those of ordinary skill in the art would recognize however, that the exact concentration of the expression construct would be dependent on the type of expression construct used, for example an adenoviral vector, and the administrative goal of the application.

Using the teachings of the specification and the knowledge of those skilled in the art, one may elect to topically deliver a nucleic acid expression construct using an adhesive bandage. An example of a nucleic acid expression construct that may be used with an adhesive bandage formulation is an adenoviral vector. In order to transduce skin by bandage, a nucleic acid expression construct formulation as a liquid suspension may be pipetted into the pad of an adhesive bandage.

The topical surface may be pretreated to enhance expression construct delivery. For example, the topical surface may be shaved to remove hair, or may be pretreated with heat, microporation, electroporation, scraping, or chemical methods. The bandage, for example, may be kept in contact with the skin for 18 hours or longer as necessary to achieve therapeutic goal.

14. Nucleic Acid Uptake Enhancers

A “nucleic acid uptake enhancer” is defined herein to refer to any agent or composition of more than one agents that can be applied to the surface of a cell or contacted with the surface of a cell to facilitate uptake of a nucleic acid that is external to the cell. Exemplary agents include cationic lipids. Cationic lipids as nucleic acid uptake enhancers are discussed in greater detail in U.S. Pat. No. 6,670,332, U.S. Pat. No. 6,399,588, U.S. Pat. No. 6,147,055, U.S. Pat. No. 5,264,618, U.S. Pat. No. 5,459,127, U.S. Pat. No. 5,994,317, and U.S. Pat. No. 5,861,397, each of which is herein specifically incorporated in its entirety. An example of a cationic lipid that can be applied in the methods and compositions of the present invention includes quaternary cytofectin (see U.S. Pat. No. 5,994,317 and U.S. Pat. No. 5,861,397.

15. Dosage

An effective amount of the therapeutic or preventive agent is determined based on the intended goal, for example (i) inhibition of growth of a hyperplastic lesion or (ii) induction of an immune response against a hyperplastic lesion.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For topical administrations, repeated application would be employed. For various approaches, delayed release formulations could be used that provide limited but constant amounts of the therapeutic agent over an extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

### J. TREATMENT OF A SURFACE OF A SUBJECT

Certain pharmaceutical compositions of the present invention are formulated for application to a surface of the subject. For example, the surface may be the skin surface, the surface of a lesion, a surgical bed following excision of a lesion, the surface of a wound, a mucosal surface, or the surface of a hollow viscus, such as the lining of the gastrointestinal tract.

A cancer may be removed by surgical excision, creating a “cavity” that has a surface. The therapeutic composition of the present invention can be administered at the time of surgery or thereafter. This is, in essence, one example of a “topical” treatment of the surface of the cavity. The volume of the composition should be sufficient to ensure that the entire surface of the cavity is contacted by the expression cassette.

In some embodiments of the methods set forth herein the pharmaceutical composition is applied using an application. Examples of applicators include sponges, swabs, cotton-tip applicators, and the like. In some embodiments, mechanical application is via a transdermal or transcutaneous delivery device may be desired. Application via swab may require one or more interactions between the swab and the topical surface. A pharmaceutical formulation of the present invention may be applied to the topical surface via a swab or sponge by repeatedly touching the swab or sponge to said surface, or by moving the swab or sponge across the surface in linear, circular or a combination of motions. Additionally a swab, sponge, transdermal or transcutaneous delivery device may be placed on the topical surface for a period of time. Any of these approaches can be used subsequent to the tumor removal as well as during the initial surgery. In still another embodiment, a catheter is inserted into the cavity prior to closure of the surgical entry site. The cavity may then be continuously perfused for a desired period of time. In still further embodiments, a pharmaceutical formulation of the present invention may be applied to a topical surface, such as the vagina or rectum, using a tampon-like applicator or a foam dispersion applicator. Methods pertaining to the use of a tampon-like applicator for delivery of pharmaceuticals is found in U.S. Pat. No. 6,588,043, methods pertaining to the use of a foam dispersion applicator is found in U.S. Pat. No. 4,112,942, each of which are specifically incorporated by reference in their entirety

In another form of this treatment, the “topical” application of the diagnostic or therapeutic composition is targeted at a natural body cavity such as the mouth, pharynx, esophagus, larynx, trachea, pleural cavity, peritoneal cavity, or hollow organ cavities including the bladder, colon, esophagus, stomach or other visceral organ. A variety of methods may be employed to affect the “topical” application into these visceral organs or cavity surfaces. For example, the oral cavity in the pharynx may be affected by simply oral swishing and gargling with mouthwashes or mouth rinses. In some applications oral swishing or gargling is contemplated to be repeated more than one time. In certain applications, the subject may hold the mouthwash or mouth rinse in the oral cavity for a period of time before spitting or swallowing. Treatment within the stomach may require an elevation in the pH of the otherwise acidic environment. However, topical treatment within the larynx and trachea may require endoscopic visualization and topical delivery of the therapeutic composition, or administration via a spray or aerosol formulation. Visceral organs such as the bladder or colonic mucosa may require indwelling catheters with infusion or again direct visualization with a cystoscope or other endoscopic instrument. Body cavities may also be accessed by indwelling catheters or surgical approaches which provide access to those areas.

In other embodiments, a topical surface may be treated or pretreated in order to increase the permeability and/or remove layers of blocking cells so as to improve nucleic acid uptake/viral infectivity. The treatment may comprise use of a wash, such as acetic acid or other membrane permeabilizing agents. Other agents include hypotonic solutions, ion chelators, cationic peptides, occludin peptides, peptides designed to disrupt extracellular portions of the junctional complexes, cytoskeletal disruption agents, antibodies, ether, neurotransmitters, glycerol, FCCP, oxidants, and mediators of inflammation. In further specific embodiments, the ion chelator may be EGTA, BAPTA or EDTA; the cationic peptide may be poly-L-lysine; the cytoskeletal disruption agent may be cytochalasin B or colchicine; the neurotransmitter may be capsianoside; the oxidant may be hydrogen peroxide or ozone; and the mediator of inflammation may be TNFα. The antibody may be an anti-E-cadherin antibody.

Alternatively, the same effect may be achieved by mechanical means. In certain embodiments the treatment may comprise scraping to remove layers of blocking cells. Sraping may involve, for example the removal of 0.1 mm to greater than 3 mm of blocking cells. Scraping of a topical surface to remove blocking cells may be accomplished with a variety of devices, such as, but not limited to a medical spatula, a needle, a dental pick, a scalpel, a knife, a dermabrasion device, or a formulation of particles suitable for dermabrasion. An example of a dermabrasion device for skin scraping is found in U.S. Pat. No. 6,629,091, which is herein incorporated by reference in its entirety.

In some embodiments, the treatment may comprise the use of lasers to ablate the topical surface of blocking cells. In certain embodiments, the treatment may comprise the use of electrodes to remove blocking cells from a topical surface. In other embodiments, the treatment may comprise the removal of blocking cells via a plasma gas electrode. In further embodiments the treatment may comprise pretreatment with an abrasive cleanser, cryotreatment, or heat. Methods and examples pertaining to ablation of blocking cells from a topical surface using lasers are found in U.S. Pat. No. 5,423,803 and U.S. Pat. No. 6,273,884, examples of blocking cell removal via electrodes are found in U.S. Pat. No. 6,024,733 and U.S. Pat. No. 6,309,387, examples pertaining to blocking cell removal via a plasma gas electrode are found in U.S. Pat. No. 6,629,974, each of which is herein incorporated by reference in its entirety. Methods pertaining to the use of heat to increase skin permeability for drug delivery may be found in U.S. Pat. No. 4,898,592.

In certain embodiments, treatment of the lung mucosa may require the use of inhaled pharmaceutical formulations in the form of sprays. In some embodiments a spray may be delivered to the lung mucosa via a nebulizer apparatus. For example, delivery of a pharmaceutical formulation of the present invention may comprise an interface for delivery into the lungs of a subject, such as a mouthpiece, a mask, an endotracheal tube, a nasal tube or the like. The interface may be connected to an inhalation tube. An inhalation tube may be connected an apparatus for providing pulsed amounts of the pharmaceutical formulation entrained in filtered atmospheric air. The apparatus may comprise a nebulizer having an inlet for pulsed air, a plenum chamber with a diffuser baffle and a connection, provided with a filter, to atmospheric air. Methods pertaining to the delivery of pharmaceutical formulations via a nebulizer may be found in, for example, U.S. Pat. No. 6,269,810 and U.S. Pat. No. 6,705,316, each of which is herein incorporated by reference in its entirety.

### K. PREVENTIVE THERAPIES

Certain embodiments of the methods set forth herein pertain to methods of preventing a disease or health-related condition in a subject. Preventive strategies are of key importance in medicine today. For example, after patients with HNSCC are cured, they have a significant (30-40%) chance of having a second primary tumor (Khuri et al., 1997). Chemoprevention of high-risk populations may reduce the development of a second primary tumor and improve survival (Khuri et al., 1997). The mucosa of the upper aerodigestive tract (UADT) is at risk for developing second primary tumors by micrometastasis (Bedi et al., 1996) or by field cancerization (Lydiatt et al., 1998). Because genetic alterations are found in histologically and clinically normal appearing mucosal tissue, these cells can progress to form a second primary tumor. These precancerous cells therefore are targets for therapeutic gene transfer. Arresting the G1-phase of the cell cycle in preneoplastic cells may halt cellular progression.

Another example of a preventative therapy is the prevention of infection or inflammation of normal tissues which can occur due to the effects of reactive oxygen species, such as those induced by radiation treatment. For example, superoxide dismutases are known to detoxify superoxide radicals to hydrogen peroxide. Methods and compositions pertaining to the delivery of nucleic acids encoding superoxide dismutases are found in, for example, U.S. Pat. No. 5,599,712, U.S. Pat. No. 6,221,712 and U.S. Pat. No. 6,887,856, each of which is specifically incorporated by reference herein in its entirety.

This same strategy can be applied to other diseases. Populations at risk can include those subjects with a risk factor or history of a disease that has been previously treated.

The quantity of pharmaceutical composition to be administered, according to dose, number of treatments and duration of treatments, depends on the subject to be treated, the state of the subject, the nature of the disease to be prevented and the protection desired. Precise amounts of the therapeutic composition also depend on the judgment of the practitioner and are peculiar to each individual. For example, the frequency of application of the composition can be once a day, twice a day, once a week, twice a week, or once a month. Duration of treatment may range from one month to one year or longer. Again, the precise preventive regimen will be highly dependent on the subject, the nature of the risk factor, and the judgment of the practitioner.

The compositions of the present invention can also be applied in immunoprophylaxis of disease in a subject, such as through vaccination or a combination of vaccination and immunotherapy. The formulations would be applied in immunization schedules known to those of ordinary skill in the art. Methods pertaining to immunoprophylaxis and vaccination are set forth in Robinson et al. (2003) and Plotkin et al. (2003), each of which is herein specifically incorporated by reference.

### L. ENHANCEMENT OF AN IMMUNE RESPONSE

In some embodiments of the methods set forth herein, a therapeutic response is obtained by enhancing an immune response in the subject. Enhancement of an immune response can be for the purpose of immune therapy of a disease or immunoprophylaxis to prevent development or progression of a disease. In certain embodiments, for example, the disease is cancer. In other embodiments, the disease is an infectious disease, or an inflammatory disease, such as an autoimmune disease.

Accordingly, in certain embodiments, a pharmaceutical formulation will be administered to a subject to enhance or induce an immune response. In certain embodiments, a therapeutic nucleic acid will encode or otherwise possess one or more immunostimulatory agent(s), such as, but not limited to antigens adjuvants and other immunomodulators.

One or more cells comprised within a target subject may express the sequences encoded by the therapeutic nucleic acid after administration of the nucleic acid to the subject. Exemplary protocols are set forth in Robinson et al. (2003) and Plotkin et al. (2003), each of which is herein specifically incorporated by reference.

In certain other embodiments, the pharmaceutical formulation itself may include one or more additional immunostimulatory agents. Still further in some embodiments, one or more of the additional agent(s) is covalently bonded to an antigen or other immunostimulatory agent, in any combination.

Antigens, may be polypeptide sequences derived from, for example, oncogenes, tumor suppressor genes, other self genes such as enzymes and genes derived from microorganisms. The nucleotide and protein, polypeptide and peptide encoding sequences for various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified, combined and/or expressed using the techniques disclosed herein or by any technique that would be know to those of ordinary skill in the art (e.g., Sambrook et al., 2001). Though a nucleic acid may be expressed in an in vitro expression system, in preferred embodiments the nucleic acid comprises a vector for in vivo replication and/or expression.

Suitable adjuvants include all acceptable immunostimulatory compounds, such as cytokines, toxins, or synthetic compositions. A non-limiting list of adjuvants that may be used in accordance with the present invention include: MDA-7, IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion, MHC antigens, complete Freund's adjuvant (a non-specific stimulator of the immune response containing killed Mycobacterium tuberculosis), incomplete Freund's adjuvant, aluminum hydroxide, Adjumer™ (i.e., PCPP salt; polyphosphazene); Adju-Phos (i.e., Aluminum phosphate gel); Algal Glucan (i.e., b-glucan; glucan); Algammulin (i.e., Gamma inulin/alum composite adjuvant); Alhydrogel (i.e., Aluminum hydroxide gel; alum); Antigen Formulation (i.e., SPT, AF); Avridine® (i.e., N,N-dioctadecyl-N′,N′-bis(2-hydroxyethyl)propanediamine; CP20,961); BAY R1005 (i.e., N-(2-Deoxy-2-L-leucylamino-b-D-glucopyranosyl)-N-octadecyldodecanoylamide hydroacetate); Calcitriol (i.e., 1a, 25-dihydroxyvitamin D3; 1,25-di(OH)2D3; 1,25-DHCC; 1a, 25-dihydroxycholecalciferol); Calcium Phosphate Gel (i.e., Calcium phosphate); Cholera holotoxin (CT) and Cholera toxin B subunit (CTB) (i.e., CT; CTB subunit; CTB); Cholera toxin A1-subunit-ProteinA D-fragment fusion protein (i.e., CTAI-DD gene fusion protein); CRL1005 (i.e., Block Copolymer P1205); Cytokine-containing Liposomes (i.e., Cytokine-containing Dehydration Rehydration Vesicles.); DDA (i.e., Dimethyldioctadecylammonium bromide; dimethyldistearylammonium bromide (CAS Registry Number 3700-67-2)); DHEA (i.e., Dehydroepiandrosterone; androstenolone; prasterone); DMPC (i.e., Dimyristoyl phosphatidylcholine; 1,2-dimyristoyl-sn-3-phosphatidyl choline; (CAS Registry Number 18194-24-6)); DMPG (i.e., Dimyristoyl phosphatidylglycerol; sn-3-phosphatidyl glycerol-1,2-dimyristoyl, sodium salt (CAS Registry Number 67232-80-8)); DOC/Alum Complex (i.e., Deoxycholic Acid Sodium Salt; DOC/Al(OH)3/mineral carrier complex); Freund's Complete Adjuvant (i.e., CIA; FCA); Freund's Incomplete Adjuvant (i.e., IFA; FIA); Gamma Inulin; Gerbu Adjuvant; GM-CSF (i.e., Granulocyte-macrophage colony stimulating factor; Sargramostim (yeast-derivedrh-GM-CSF)); GMDP (i.e., N-acetylglucosaminyl-(b1-4)-N-acetylmuramyl-L-alanyl-D-isoglutamine (CAS Registry Number 70280-03-4)); Imiquimod (i.e., 1-(2-methypropyl)-1H-imidazo[4,5-c]quinolin-4-amine; R-837; S26308); ImmTher™ (i.e., N-acetylglucosaminyl-N-acetyhnuramyl-L-Ala-D-isoGlu-L-Ala-glycerol dipalmitate; DTP-GDP); Immunoliposomes Containing Antibodies to Costimulatory Molecules (i.e., Immunoliposomes prepared from Dehydration-Rehydration Vesicles (DRVs)); Interferon-g (i.e., Actimmune® (rhIFN-gamma, Genentech, Inc.); immune interferon; IFN-g; gamma-interferon); Interleukin-1b (i.e., IL-10; IL-1; human Interleukin 1b mature polypeptide 117-259); Interleukin-2 (i.e., IL-2; T-cell growth factor; aldesleukin (des-alanyl-1, serine-125 human interleukin 2); Proleukin®; Teceleukin®); Interleukin-7 (i.e., IL-7); Interleukin-12 (i.e., IL-12; natural killer cell stimulatory factor (NKSF); cytotoxic lymphocyte maturation factor (CLMF)); ISCOM(s)™ (i.e., Immune stimulating complexes); Iscoprep 7.0.3.™; Liposomes (i.e., Liposomes (L) containing protein or Th-cell and/or B-cell peptides, or microbes with or without co-entrapped interleukin-2, Bis HOP or DOTMA; A, [L (Antigen)]); Loxoribine (i.e., 7-allyl-8-oxoguanosine); LT-OA or LT Oral Adjuvant (i.e., E. coli labile enterotoxin protoxin); MF59; MONTANIDE ISA 51 (i.e., Purified IFA; Incomplete Freund's adjuvant.); MONTANIDE ISA 720 (i.e., metabolizable oil adjuvant); MPL™ (i.e., 3-Q-desacyl-4′-monophosphoryl lipid A; 3D-MLA); MTP-PE (i.e., N-acetyl-L-alanyl-D-isoglutaminyl-L-alanine-2-(1,2-dipalmitoyl-sn-glycero-3-(hydroxy-phosphoryloxy)) ethylamide, mono sodium salt); MTP-PE Liposomes (i.e., MTP-PE Antigen presenting liposomes); Murametide (i.e., Nac-Mur-L-Ala-D-Gln-OCH3); Murapalmitine (i.e., Nac-Mur-L-Thr-D-isoGln-sn-glycerol dipalmitoyl); D-Murapalmitine (i.e., Nac-Mur-D-Ala-D-isoGln-sn-glycerol dipalmitoyl); NAGO (i.e., Neuraminidase-galactose oxidase); Non-Ionic Surfactant Vesicles (i.e., NISV); Pleuran (i.e., b-glucan; glucan); PLGA, PGA, and PLA (i.e., Homo- and co-polymers of lactic and glycolic acid; Lactide/glycolide polymers; poly-lactic-co-glycolide); Pluronic L121 (i.e., Poloxamer 401); PMMA (i.e., Polymethyl methacrylate); PODDS™ (i.e., Proteinoid microspheres); Poly rA:Poly rU (i.e., Poly-adenylic acid-poly-uridylic acid complex); Polysorbate 80 (i.e., Tween 80; Sorbitan mono-9-octadecenoate poly(oxy-1,2-ethanediyl) derivatives); Protein Cochleates; QS-21 (i.e., Stimulon™ QS-21 Adjuvant); Quil-A (i.e., Quil-A saponin, Quillaja saponin); Rehydragel HPA (i.e., High Protein Absorbency Aluminum Hydroxide Gel; alum); Rehydragel LV (i.e., low viscosity aluminum hydroxide gel; alum); S-28463 (i.e., 4-Amino-otec,-dimethyl-2-ethoxymethyl-1H-imidazo[4,5-c]quinoline-1-ethanol); SAF-1 (i.e., SAF-m; Syntex Adjuvant Formulation); Sclavo peptide (i.e., IL-1b 163-171 peptide); Sendai Proteoliposomes, Sendai-containing Lipid Matrices (i.e., Sendai glycoprotein-containing vesicles; fusogenic proteoliposomes; FPLS); Span 85 (i.e., Arlacel 85, sorbitan trioleate); Specol; Squalane (i.e., Spinacane; Robane®; 2,6,10,15,19,23-hexamethyltetracosane); Squalene (Spinacene; Supraene; 2,6,10,15,19,23-hexamethyl-2,6,10,14,18,22 tetracosahexaene); Stearyl Tyrosine (i.e., Octadecyl tyrosine hydrochloride); Theramide™ (i.e., N-acetylglucosaminyl-N-acetylinuramyl-L-Ala-D-isoGlu-L-Ala-dipalmitoxy propylamide (DTP-DPP)); Threonyl-MDP (i.e., Termurtide™; [thrl]-MDP; N-acetyl muramyl-L-threonyl-D-isoglutamine); Ty Particles (i.e., Ty-VLPs, (Virus Like Particles)); Walter Reed Liposomes (i.e., Liposomes containing lipid A adsorbed to aluminum hydroxide, [L(Lipid A+Antigen)+Alum]).

In addition to adjuvants, it may be desirable to administer immunomodulators, such as antisense RNA, RNAi, nucleic acids encoding Cpg motifs and biological response modifiers (BRMs) which have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m2) (Johnson/Mead, NJ) and cytokines such as g-interferon, IL-2, or IL-12 or genes encoding proteins involved in immune helper functions, such as B-7.

In further embodiments of the present invention, the nucleic acid encoding or otherwise possessing one or more immunostimulatory agent(s) can be administered to a subject such that the expression of the nucleic acid may induce a humoral or cell mediated immune response in a subject.

The immune response may be an active or a passive immune response. Alternatively, the response may be part of an adoptive immunotherapy approach in which lymphocyte(s) are obtained with from an animal (e.g., a patient), then pulsed with composition comprising an antigenic composition. In this embodiment, the antigenic composition may comprise an additional immunostimulatory agent or a nucleic acid encoding such an agent. The lymphocyte(s) may be obtained from the blood of the subject, or alternatively from tumor tissue to obtain tumor infiltrating lymphocyte(s) as disclosed in Rosenberg et al., 1986, incorporated herein by reference. In particular embodiments, the lymphocyte(s) are peripheral blood lymphocyte(s). In one particular embodiment, the lymphocyte(s) can be administered to the same or different animal (e.g., same or different donors). For example, the animal (e.g., a patient) may have or is suspected of having a cancer, such as a breast or prostate cancer. In other embodiments the method of enhancing the immune response is practiced in conjunction with a cancer therapy, such as for example, a cancer vaccine therapy, as discussed in greater detail below.

One or more cells comprised within a target subject may express the sequences encoded by the nucleic acid after administration of the nucleic acid to the subject. Exemplary protocols are set forth in Robinson et al. (2003) and Plotkin et al. (2003), each of which is herein specifically incorporated by reference.

Examples of suitable tumor antigens are known to those of ordinary skill in the art including but not limited to those described by Dalgleish, 2004; Finn, 2003; and Hellstrom and Hellstrom, 2003. Each of which is herein incorporated by reference in its entirety.

Topical application of nucleic acids encoding tumor antigens to mucosal surfaces may be contemplated as prophylactic or preventative therapies Accordingly such mucosal application may generate an immunoprotective effect against subsequent development of hyperproliferative diseases such as cancer.

In some embodiments, it is contemplated that nucleic acids encoding tumor antigens may be applied to mucosal surfaces prior to the development of a hyperproliferative disease such as cancer. Mucosal application of compositions containing one or more antigen(s) derived from microorganisms has been previously reported. These studies indicate that mucosal application of such antigens may induce a prophylactic immune response against microorganisms which infect such surfaces. (Gallichan et al., 1993; Gallichan and Rosenthal, 1995; Gallichan and Rosenthal, 1996.) Conversely, it has been reported that mucosal application of such antigens subsequent to an established infection may decrease or abrogate a meaningful therapeutic benefit. For example, currently available polio and pneumoccocal vaccines administered after establishment of infection may not be therapeutically effective compared to administration prior to exposure to these microorganisms.

### M. SECONDARY FORMS OF THERAPY

1. General

In certain embodiments of the present invention, the methods of the present invention pertain to detection, treatment or prevention of disease in a subject, wherein the subject one or more secondary forms of therapy.

Certain aspects of the present invention pertain to methods of administering a modulator of human ACC to a subject, such as a human subject. These compositions can be applied in the prevention or treatment of diseases wherein administration of a modulator of human ACC is known or suspected by one of ordinary skill in the art to be beneficial.

For example, as set forth above, the disease or health-related condition to be treated or prevented may be obesity, a hyperproliferative disease, a cardiovascular disease, diabetes, or insulin resistance. The modulator of human ACC may be administered along with another agent or therapeutic method. For example, administration of a modulator of human ACC for the purpose of treating diabetes mellitus in a human subject may precede, follow, or be concurrent with other therapies for diabetes, such as an oral hypoglycemic acid or insulin therapy. Administration of a modulator of human ACC for the purpose of treating an acute myocardial infarction may, for example, be administered following an angioplasty or coronary artery bypass procedure. In another example, administration of a modulator of human ACC of the purpose of treating or prevent obesity may precede or follow a dietary intervention or gastric surgery for the treatment of obesity.

Administration of the modulator of human ACC to a patient will follow general protocols for the administration of therapeutic agents, and will take into account other parameters, including, but not limited to, other medical conditions of the patient and other therapies that the patient is receiving. It is expected that the treatment cycles would be repeated as necessary.

Treatment with the modulator of human ACC of the present invention may precede or follow the other therapy method by intervals ranging from minutes to weeks. In embodiments where another agent is administered, one would generally ensure that a significant period of time did not expire between the time of each delivery, such that the agents would still be able to exert an advantageously combined effect on the cell. For example, it is contemplated that one may administer two, three, four or more doses of one agent substantially simultaneously (i.e., within less than about a minute) with the compositions of the present invention. In other aspects, a therapeutic agent or method may be administered within about 1 minute to about 48 hours or more prior to and/or after administering a therapeutic amount of a composition of the present invention, or prior to and/or after any amount of time not set forth herein. In certain other embodiments, the modulator of human ACC of the present invention may be administered within of from about 1 day to about 21 days prior to and/or after administering another therapeutic modality, such as surgery or medical therapy. In some situations, it may be desirable to extend the time period for treatment significantly, however, where several weeks (e.g., about 1 to 8 weeks or more) lapse between the respective administrations.

Various combinations may be employed, the modulator of human ACC is designated “A” and the secondary therapeutic agent, which can be any other therapeutic agent or method, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

2. Secondary Anti-Cancer Therapies

A wide variety of cancer therapies, known to one of skill in the art, may be used in combination with the compositions of the claimed invention. Some of the existing cancer therapies and chemotherapeutic agents are described below. One of skill in the art will recognize the presence and development of other anticancer therapies which can be used in conjugation with the methods and compositions of the present invention, and will not be restricted to those forms of therapy set forth below.

In order to increase the effectiveness of a therapeutic nucleic acid, it may be desirable to combine it with one or more other agents or modalities effective in the treatment of hyperproliferative disease. Therapeutic compositions may be combined or administered separately. The therapeutic goal would be to kill or inhibit proliferation of cancerous cells. This process may involve contacting the cells with the expression construct and the agent(s) or second factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent.

Alternatively, the nucleic acid therapy may precede or follow the other agent or modality by intervals ranging from minutes to weeks. In embodiments where the other agent and expression construct are applied separately, one would generally ensure that a significant period of time did not expire between the time of each delivery, such that the agent and expression construct would still be able to exert an advantageously combined therapeutic effect. In such instances, it is contemplated that one may contact the cell with both forms of therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, for example, the primary therapy is “A” and the secondary is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic nucleic acids of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Radiotherapy

Radiotherapy include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In the context of the present invention radiotherapy, radiotherapy may be performed before, during, or after treatment with one of the therapeutic nucleic acids set forth herein, and may be repeated as per standard protocols.

b. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, in the context of the present invention surgery may be used in addition to using the tumor cell specific-peptide of the invention to achieve cell-specific cancer therapy.

In the case of surgical intervention, the compositions of the present invention may be used preoperatively, to render an inoperable tumor subject to resection. Alternatively, the present invention may be used at the time of surgery, and/or thereafter, to detect or treat residual or metastatic disease. For example, a resected tumor bed in the oral cavity of a subject may be detected or treated by application of one of the pharmaceutical compositions of the present invention. The applications may be continued post-resection. Periodic post-surgical treatment also is envisioned.

In certain embodiments, the tumor being treated may not, at least initially, be respectable. Treatments with diagnostic or therapeutic viral constructs may increase the respectability of the tumor due to shrinkage at the margins or by elimination of certain particularly invasive portions. Furthermore, a viral construct encompassing a reporter gene with the ability to cause color changes in a specific tissue type may aid in surgical removal of hyperproliferative cells. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher.

c. Chemotherapeutic Agents

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin, benzimidazoles, and methotrexate or any analog or derivative variant thereof. The term “chemotherapy” as used herein is defined as use of a drug, toxin, compound, composition or biological entity which is used as treatment for cancer. These can be, for example, agents that directly cross-link DNA, agents that intercalate into DNA, agents that can disrupt the microtubule system, drugs that cause accumulation of tumor suppressor proteins and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

Agents that disrupt the microtubule system of cells include for example benzimidazoles. Benzimidazoles are a broad-spectrum class of antihelmintics that display excellent activity against parasitic nematodes and, to a lesser extent, against cestodes and trematodes. Benzimidazoles have also been shown to be very effective antiprotozoal agents that also have antifungal activity. It is currently believed that benzimidazoles exert their cytotoxic effects by binding to the microtubule system and disrupting its functions (Lacey, 1988; Friedman and Platzer, 1980). The suggestions that tubulin is a target for benzimidazoles has been supported by the results of drug-binding studies using enriched extracts of helminth and mammalian tubulin (Lacey, 1988). Moreover, competitive drug-binding studies using mammalian tubulin have shown that benzimidazoles compete for colchicine binding and inhibit growth of L1210 murine leukemia cells in vitro (Friedman and Platzer, 1978; Lacey and Watson, 1989). However, benzimidazoles are selectively toxic to nematodes when administered as antihelmintics but are not toxic to the host. In contrast, benzimidazoles suppress the in vitro polymerization of mammalian tubulin. Differences in both the affinity between the host and parasite macromolecules for benzimidazoles (Russell et al., 1992; Kohler and Bachmann, 1981) and the pharmacokinetics of benzimidazoles between the host and the parasite have been suggested as responsible for the selective toxicity of benzimidazoles (Gottschall et al., 1990) but the actual molecular basis of this selective toxicity remains unclear.

Mebendazole, or 5-benzoyl-2-benzimidazole carbamic acid methyl ester, is a member of the benzimidazole class of compounds. Recently, mebendazole has been found to induce mitotic arrest and apoptosis by depolymerizing tubulin in non-small cell lung cancer cells. (Sasaki et al., 2002). mebendazole has also been found to elicit a potent antitumor effect on human cancer cell lines both in vitro and in vivo (Mukhopadhyay et al., 2002).

Mebendazole was first introduced for the treatment of roundworm infections as a result of research carried out by Brugmans et al. (1971). It is the prototype of a series of broad-spectrum anthelmintics widely used in both animals and man (Michiels et al., 1982) as broad-spectrum anthelmintics for animal and human use (Van den Bossche et al., 1982). Related benzimidazole derivatives with anthelmintic properties include albendazole and flubendazole. Alternative benzimidazoles are: fenbendazole, albendazole, albendazole sulfone, oxibendazole, rycobendazole, thiabendazole, oxfendazole, flubendazole and carbendazim.

Mebendazole causes selective disappearance of cyoplasmic microtubules in the tegumental and intestinal cells of affected worms. Secretory substances accumulate in Golgi areas, secretion of acetylcholinesterase and uptake of glucose are impaired, and glycogen is depleted. These effects of mebendazole are not noted in host cells. Mebendazole has a high affinity for parasite tubulin in vitro, but it also binds to host tubulin. The biochemical basis for its selective action is thus unclear (see Van den Bossche, 1981; Watts et al., 1982).

Mebendazole is highly lipophilic, with an aqueous solubility of less than 1 μg/ml. As a result tablets of MZ are poorly and erratically absorbed, and concentrations of the drug in plasma are low and do not reflect the dosage taken (Witassek et al., 1981). Thus, conventional formulations of mebendazole result in low bioavailability of the drug and erratic absorption from the gastrointestinal tract. Many other benzimidazoles and benzimidazole derivatives are also highly lipophilic and erratically absorbed from the gastrointestinal tract. As a result, benzimidazoles may be advantageous in pharmaceutical formulations which contemplate oral or topical application.

It is contemplated that routes of administration for the various chemotherapies described herein may be administered through various routes such as, but not limited to: intradermally, parenterally, intravenously, intramuscularly, intranasally, and orally and topically.

d. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with methods set forth herein. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

e. Genes

In yet another embodiment, the secondary treatment is an additional gene therapy in which an additional form of therapeutic nucleic acid (for example, a formulation of a nucleic acid for intravenous delivery) is administered before, after, or at the same time as the pharmaceutical compositions set forth herein. Thus, for example, the present invention contemplates that a subject may be treated using more than one of the methods set forth herein for the delivery of a therapeutic or preventive nucleic acid sequence. In some embodiments, a single vector encoding both genes may be used.

f. Other Cancer Therapies

Examples of other cancer therapies include phototherapy, cryotherapy, toxin therapy, or hormonal therapy. One of skill in the art would know that this list is not exhaustive of the types of treatment modalities available for cancer and other hyperplastic lesions.

### N. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of P53 Expression Vector

This example pertains to exemplary techniques for construction of a p53 expression vector. This vector is constructed as indicated and is used to replace the E1 region (1.3-9.2 m.u.) of the Adenovirus strain Ad5 genome and employed to construct the Adenovirus virion described below in Example 2.

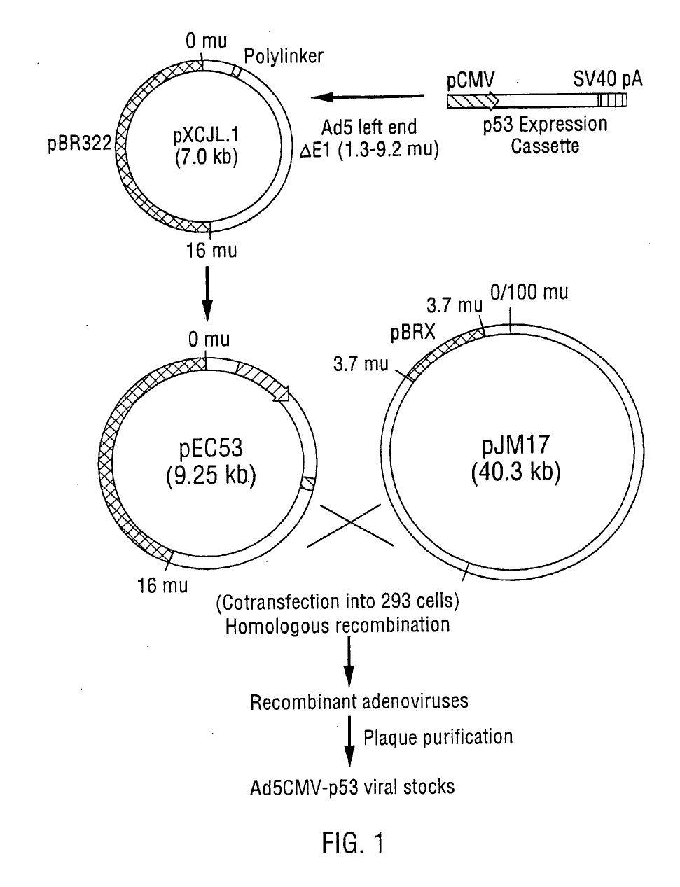

The p53 expression cassette shown in depicted in FIG. 1, which contains human cytomegalovirus (CMV) promoter (Boshart et al., 1985), p53 cDNA, and SV40 early polyadenylation signal, was inserted between the Xba I and Cla I sites of pXCJL1 (provided by Dr. Frank L. Graham, McMaster University, Canada). The genome size is about 35.4 kb, divided into 100 map units (1 m.u.=0.35 kb). The p53 expression cassette replaced the E1 region (1.3-9.2 m.u.) of the Ad5 genome.

Primer 1 has the sequence 5′-GGCCCACCCCCTTGGCTTC-3′ (SEQ ID NO:1) and is located in the first intron downstream of the human CMV major IE gene promoter (Boshart et al., 1985). Primer 2 has the sequence 5′-TTGTAACCATTATAAGCTGC-3′ (SEQ ID NO:2) and is located in SV40 early polyadenylation signal. Both of the primers, 15-20 bp away from the p53 cDNA insert at both ends, define a 1.40 kb PCR product. Primer 3 has the sequence 5′-TCGTTTCTCAGCAGCTGTTG-3′ (SEQ ID NO:3) and primer 4 has the sequence 5′-CATCTGAACTCAAAGCGTGG-3′ (SEQ ID NO:4) and are located at 11 m.u. and 13.4 m.u. of the Ad5 genome, respectively, which define a 0.86 kb viral-genome specific PCR product. Other methods for constructing such vectors that employ variations of this method can be applied in construction of a p53 expression vector.

### Example 2

### Generation and Propagation of Recombinant p53 Adenovirus

This example describes one exemplary method suitable for generating helper-independent recombinant adenoviruses expressing p53. The molecular strategy employed to produce recombinant adenovirus is based upon the fact that, due to the packaging limit of adenovirus, pJM17 cannot form virus on its own. Therefore, homologous recombination between the p53 expression vector plasmid and pJM17 within a transfected cell results in a viable virus that can be packaged only in cells which express the necessary adenoviral proteins.

The method of this example utilizes 293 cells as host cells to propagate viruses that contain substitutions of heterologous DNA expression cassettes at the E1 or E3 regions. This process requires cotransfection of DNA into 293 cells. The transfection largely determines efficiency of viral propagation. The method used for transfection of DNA into 293 cells prior to the present invention was usually calcium-phosphate/DNA coprecipitation (Graham and van der Eb, 1973). However, this method, together with the plaque assay, is relatively difficult and typically results in low efficiency of viral propagation. As illustrated in this example, transfection and subsequent identification of infected cells were significantly improved by using liposome-mediated transfection, when identifying the transfected cells by cytopathic effect (CPE).

The 293 cell line was maintained in Dulbecco's modified minimal essential medium supplemented with 10% heat-inactivated horse serum. The p53 expression vector and the plasmid pJM17 (McGrory, et al., 1988) for homologous recombination were cotransfected into 293 cells by DOTAP-mediated transfection according to the manufacture's protocol (Boehringer Mannheim Biochemicals, 1992). This is schematically shown in FIG. 1.

The 293 cells (passage 35, 60% confluency) were inoculated 24 hours prior to the transfection in either 60 mm dishes or 24-well plates. The cells in each well were transfected with: 30 .mu.l DOTAP, 2 .mu.g of p53 expression vector, and 3 .mu.g of plasmid pJM17. After transfection cells were fed with the MEM medium every 2-3 days until the onset of CPE. Other methods for generating and propagating recombinant adenoviral vectors using variations of these techniques and/or other techniques well-known to those of ordinary skill in the art can be employed.

### Example 3

### In Vivo Detection of Tumors with Optical Imaging by Telomerase-Specific Amplification of a Transferred Green Fluorescent Protein Gene

This example sets forth an exemplary protocol for in vivo studies that can be conducted to determine the ability of nucleic acid expression constructs encoding a reporter gene such as green fluorescent protein gene (gfp) to detect tumors in murine models. In an initial round of in vivo trials, BALB/c nu/nu mice subcutaneously injected with human lung and colon cancers (Umeoka et al., 2004) can be used. For example, animals may be treated with nucleic acid expression constructs encoding the gfp capable of expression only in cells expressing human telomerase reverse transcriptase, which is active in >85% of human cancer cells but not in most normal cells. Accordingly, an hTERT promoter may be preferable as a tissue selective promoter to drive expression of gfp as the normal product of hTERT expression is human telomerase reverse transcriptase.

For example, nucleic acid expression constructs encoding gfp under operative control by the hTERT promoter can be tested in vivo for tumor detection in antitumor activity in BALB/c nu/nu mice subcutaneously injected with human lung and colon cancers.

The effect of nucleic acid expression constructs encoding gfp under operative control by the hTERT promoter can then be assessed by optical examination of tumor tissue samples under fluorescent microscope, for instance, an Eclipse TS-100 fluorescent microscope (Nikon, Tokyo, Japan).

### Example 4

### In Vivo Prevention of Tumor Development of the Stomach Using a Nucleic Acid Expression Construct Encoding a Tumor Suppressor Gene

This example sets forth examples of in vivo studies that can be conducted to determine the ability of nucleic acid expression constructs encoding tumor suppressor genes to inhibit cancer in murine models. In an initial round of in vivo trials, a mouse model of human stomach and esophageal cancer (Dumon et al., 2001) can be used. For example, Fhit^{-/-} mice are susceptible to carcinogen induced tumor development in the esophagus and forestomach after exposure to the carcinogen N-nitrosomethylbenzylamine (NMBA). The animals may be treated with nucleic acid expression constructs encoding the human FHIT tumor suppressor gene to determine the suppression of tumor development.

For example, nucleic acid expression constructs encoding the human FHIT tumor suppressor gene can be tested in vivo for antitumor activity in Fhit^{-/-} mice exposed to NMBA, or any other murine model of cancer known to those of skill in the art In conjunction with these studies, the antitumor activity of nucleic acid expression constructs encoding the human FHIT tumor suppressor gene can be assessed in a murine model.

In brief, different groups of mice of a suitable cancer model can be treated with doses of nucleic acid expression constructs encoding the human FHIT tumor suppressor gene after pretreatment with a carcinogen such as NMBA. Several combinations and concentrations nucleic acid expression constructs encoding the human FHIT tumor suppressor gene can be tested. Control mice should only be pretreated with NMBA.

The effect of nucleic acid expression constructs encoding the human FHIT tumor suppressor gene on the development of cancer in treated mice versus a control group can then be compared by examination of tumor size and histopathologic examination of hematoxylin and eosin stained tumor tissue. Immunohistochemical examination may also be performed by incubation of the sample tissue with rabbit anti-human Fhit antibody against the C terminus of the human Fhit protein followed by incubation with biotinylated goat anti-rabbit antibody.

### Example 5

### AdCMV-p53: Single-Dose Oral Biodistribution Study in Mice with a 2-Week Observation Period

Procedure

Biodistribution of AdCMV-p53 was evaluated in C57BL/6N mice following a single oral gavage dose of 8.3×10¹⁰ (Group 2), 8.3×10¹¹ (Group 3) or 8.3×10¹² (Group 4) vp/kg. Each treatment group consisted of six male and six female mice; a control group (Group 1) of the same size received only vehicle. On day 4 or 15 after treatment, tissue samples were collected in the following order: ovaries/testes, liver, kidney, adrenals, spleen, stomach, lymph node, ileum, rectum, heart, lung, esophagus, muscle, bone femur, brain and spinal cord. Tissues were snap-frozen in liquid nitrogen and stored at −70±10° C. Blood samples were drawn from the retro-orbital sinus into sterile EDTA blood collector tubes, stored at 4±2° C. and processed for DNA extraction within 3 days.

Genomic DNA was isolated from frozen tissue samples. Each set of DNA extractions included all tissues from a single animal. Extractions were performed on tissues from Group 1 (control) animals first, followed by extraction of tissues from Groups 2, 3, and 4. Tissue and blood DNA samples were quantified by absorbance at 260 nm and stored below −15° C. until use.

Quantitative PCR analyses were conducted using Real-Time PCR (Taqman® PCR). Primers yielded a 70 bp amplification product encompassing the junction between the CMV promoter and the untranslated p53 5′ region.

| | | |
|---|---|---|
| PREYF: | 5′ TTATGCGACGGATCCCGTAA 3′ | (SEQ ID NO:5) |
| PREYR: | 5′ GCGTGTCACCGTCGTACGTA 3′ | (SEQ ID NO:6) |
| Probe: | 5′ CTTCGAGGTCCGCGGCCG 3′ | (SEQ ID NO:7) |

Assay sensitivity was 100 vector DNA copies in 0.5 μg of mouse genomic DNA, and was linear over a template range spanning from 10⁰ to 10⁵ copies.

Each 96-well PCR reaction plate contained a negative control containing no DNA to verify the absence of contamination, and a series of ten-fold dilutions of AdCMV-p53 DNA to generate a standard curve. Each PCR reaction was performed in duplicate, one of which was spiked with AdCMV-p53 DNA to verify the absence of PCR inhibitors.

Quantitation of positive samples was performed by plotting the un-spiked samples on the standard curve. Results of PCR analysis were reported as copy number/0.5 μg of mouse tissue DNA. Samples with values greater than 10 copies were considered positive. However, since detection of 10 copies was not consistently achieved, values between 10 and 100 copies may not be precise, since they are interpolated by the ABI 7700 based on the standard curve.

Results

Real-Time PCR analysis consistently detected 100 copies of AdCMV-p53 DNA in 0.5 μg DNA. Vector DNA levels from 10-100 copies/0.5 μg DNA were considered low (and were not consistently detected), 100-1000 copies/0.5 μg DNA intermediate, and above 1000 copies/0.5 μg DNA high. Ad5CMV-p53 DNA levels below 10 copies/0.5 μg DNA were defined as non-quantifiable, as false-negative may arise from the random assortment of the few copies in a sample.

In the high-dose group (8.3×10¹² vp/kg), with tissue and blood samples collected on day 4 after dosing, AdCMV-p53 DNA was detected at intermediate levels or higher (over 100 copies per 0.5 μg DNA) in the liver, stomach, lungs, esophagus, muscle, brain, spinal cord, and blood of at least one animal (Table 9). By day 15, only lung samples from the high-dose group remained positive at or above intermediate levels.

In the mid-dose group (8.3×10¹¹ vp/kg) on day 4, samples from the stomach, lungs, esophagus, and blood were positive at intermediate levels or above. Samples from mid-dose animals with AdCMV-p53 DNA present at or above intermediate levels were found in the adrenal, heart, lungs, esophagus, muscle, and spinal cord by day 15.

In the low-dose group (8.3×10¹⁰ vp/kg), only samples from the lungs and blood tested positive for AdCMV-p53 DNA at or above intermediate levels on day 4. Samples from the lungs, esophagus, and bone were positive at or above intermediate levels on day after low-dose AdCMV-p53. No quantifiable signal was detected in any of the control samples. The tables below list both the average amount of AdCMV-p53 DNA in the various organs and tissues, and the number of samples that were positive at >10 copies of AdCMV-p53 per 0.5 μg mouse genomic DNA.

The vast majority of positive samples were sporadic. The only organs in which all samples at a given dose and time point were positive was blood in mid-dose animals (approximately 200 copies/0.5 ug). Organs in which ≧4 of 6 samples were positive were all in the high-dose group: lung (240,000 copies/0.5 ug), esophagus (900 copies/0.5 ug), blood (250 copies/0.5 ug), and stomach (110 copies/0.5 ug).

### CONCLUSIONS

After a single oral dose of AdCMV-p53, the AdCMV-p53 DNA is primarily located in the lungs and esophagus. The appearance of AdCMV-p53 DNA was sporadic or negative in most organs at day 4, with the exception of blood, lungs, and esophagus. At day 15, in low- and mid-dose animals, more organs were positive for Ad5CMV-p53 DNA than at day 4. In the high-dose animals, the number of positive organs, and the absolute titers of AdCMV-p53 DNA in an organ, decreased from day 4 to day 15.

The dose- and time-dependence of AdCMV-p53 DNA PCR signal strength in this study did not follow the trends seen in most of the other biodistribution studies (greater signal strength at higher doses and shorter times). First, AdCMV-p53 DNA was detected in more organs at day 15 than at day 4 (in low- and mid-dose groups), suggesting a slow dissemination with an oral route of administration. Second, the levels and dissemination of AdCMV-p53 DNA was dose-dependent at day 4, but not at day 15. At day 15, the amount of AdCMV-p53 DNA was lower in organs from high-dose animals than in organs from mid-dose animals (with the exception of the lung), and was even lower in many organs from high-dose animals than in organs from low-dose animals.

TABLE 9
Biodistribution of AdCMV-p53 DNA in Mice after
Oral Administration of AdCMV-p53⁺

| | Average # of copies of | | | |
|---|---|---|---|---|
| | ADVEXIN DNA (# of ADVEXIN | | | |
| | copies/0.5 μg DNA)^{#} | | | |
| | Group 1 | Group 2 | Group 3 | Group 4 |
| Organ | (Control) | (Low Dose) | (Mid Dose) | (High Dose) |

Day 4

| | | | | |
|---|---|---|---|---|
| Ovaries/Testes | 0 | 9* | 0 | 2* |
| | | (1 ovary) | | (1 ovary) |
| Liver | 0 | 0 | 8* | 31 |
| Kidney | 0 | 0 | 0 | 0 |
| Adrenals | 0 | 15* | 0 | 0 |
| Spleen | 0 | 0 | 0 | 5* |
| Stomach | 0 | 0 | 45* | 110 |
| Lymph node | 0 | 0 | 0 | 2* |
| Ileum | 0 | 0 | 0 | 0 |
| Rectum | 0 | 0 | 0 | 0 |
| Heart | 0 | 0 | 0 | 0 |
| Lungs | 0 | 1400* | 1.2 × 10^{4*} | 2.4 × 10⁵ |
| Esophagus | 2* | 9* | 70* | 880 |
| Muscle | 0 | 0 | 2* | 93* |
| Bone femur | 2* | 2* | 0 | 0 |
| Brain | 0 | 5* | 7* | 51* |
| Spinal Cord | 0 | 0 | 0 | 61* |
| Blood | 0 | 70* | 210 | 250 |

Day 15

| | | | | |
|---|---|---|---|---|
| Ovaries/Testes | 0 | 2* | 36 | 6* |
| | | (1 ovary) | (1 ovary) | |
| Liver | 0 | 3* | 10 | 0 |
| Kidney | 0 | 5* | 35 | 0 |
| Adrenals | 0 | 5* | 47 | 0 |
| Spleen | 0 | 0 | 15* | 0 |
| Stomach | 0 | 5* | 15 | 0 |
| Lymph node | 0 | 4* | 3* | 0 |
| Ileum | 0 | 0 | 16 | 2* |
| Rectum | 0 | 12* | 17 | 0 |
| Heart | 0 | 6* | 63 | 0 |
| Lungs | 0 | 42 | 43 | 530 |
| Esophagus | 0 | 37 | 130 | 13* |
| Muscle | 0 | 20 | 120 | 0 |
| Bone femur | 0 | 28 | 15 | 0 |
| Brain | 0 | 16* | 26 | 0 |
| Spinal Cord | 0 | 16 | 440 | 19* |
| Blood | 0 | 3* | 2* | 0 |

⁺Samples were analyzed by quantitative Real-Time PCR.
^{#}Non-quantifiable samples (“NQ”) were defined as 10 copies/0.5 μg for the purposes of this table.
*Only 1 or 2 of the samples assayed gave a copy number of 10 or more; the remaining samples were negative.

### Example 6

### Assays to Assess the Efficacy of Formulations of Therapeutic or Diagnostic Nucleic Acids

Using the teachings of the specification and the knowledge of those skilled in the art, one can conduct studies to assess the efficacy of various formulations of nucleic acids. One of ordinary skill in the art would understand that the effectiveness of a formulation of a particular nucleic acid as a therapeutic or detectable agent depends on many factors, such as the concentration of the nucleic acid, the pH, the temperature of the formulation, other constituents of the formulation, and so forth.

For example, various formulations of a particular nucleic acid, in which any or all of these factors are varied, can be examined for therapeutic efficacy by any of a number of techniques known to those of ordinary skill in the art. For example, if the disease to be treated or prevented is a hyperproliferative disease such as cancer, the therapeutic efficacy of these formulations can be evaluated using an appropriate in vivo model of human cancer, such as a nude mouse with implanted tumor cells. For example, it can be determined whether a particular formulation demonstrates efficacy in reducing the size of tumors in animal models. Frequency and method of application of the formulation can be evaluated in the animal model. Therapeutic response, as well as presence or absence of side effects can be evaluated using information well-known to those of ordinary skill in the art.

Regarding diagnostic nucleic acids, such as nucleic acids encoding reporter proteins, studies to evaluate the presence or absence of detectable protein in the cells of the animal model can be conducted using any of a number of techniques well-known to those of ordinary skill in the art. For example, optical imaging using techniques such as those set forth in Example 4 can be performed and compared to appropriate controls.

### Example 7

### Clinical Trials of the Use of Nucleic Acid Formulations for Topical Delivery in the Treatment of Diseases—General Considerations

This example is generally concerned with the development of human treatment protocols using the nucleic acid formulations of the present invention. In particular, such treatment can be of use in the therapy of various diseases in which administration of a nucleic acid is known or considered to be of benefit. Examples of these diseases include treatment of hyperproliferative diseases such as cancer, wound healing, and treatment of infections. A more detailed example pertaining to cancer is discussed in the next example.

The various elements of conducting a clinical trial, including patient treatment and monitoring, will be known to those of skill in the art in light of the present disclosure. The following information can be used as a general guideline for use in establishing use of nucleic acid formulations in clinical trials.

Patients with the targeted disease can be newly diagnosed patients or patients with existing disease. Patients with existing disease may include those who have failed to respond to at least one course of conventional therapy.

The nucleic acid formulation may be administered alone or in combination with another therapeutic agent. The therapeutic nucleic acid may be administered in accordance with any of the methods set forth in this specification, such as topical application and oral administration. The agent may be administered during the course of a procedure, such as surgical excision to remove diseased tissue.

The starting dose may, for example, be 0.5 mg/kg body weight. Three patients may be treated at each dose level in the absence of a defined level of toxicity. Dose escalation may be done by 100% increments (e.g., 0.5 mg, 1 mg, 2 mg, 4 mg) until drug related toxicity of a specific level develops. Thereafter dose escalation may proceed by 25% increments. The administered dose may be fractionated.

The nucleic acid formulation may be administered, for example, a single time, or multiple times over a period of days or weeks. Administration may be alone or in combination with other agents.

Physical examination, laboratory tests, and other clinical studies specific to the disease being treated may, for example, be performed before treatment and at intervals of about 3-4 weeks later. Laboratory studies can include CBC, differential and platelet count, urinalysis, SMA-12-100 (liver and renal function tests), coagulation profile, and any other appropriate chemistry studies to determine the extent of disease, or determine the cause of existing symptoms.

Response to therapy can be in accordance with any method known to those of ordinary skill in the art, and are largely dependent upon the disease to be treated. For example, when the disease is cancer, response can be assessed by decrease in size of a tumor. Wound healing can be assessed by evaluating wound size and/or clinical appearance.

### Example 8

### Clinical Trials of the Use of Nucleic Acid Formulations for Topical or Oral Delivery in the Treatment of Cancer

This example describes an exemplary protocol that might be applied in the treatment of human cancer patients using the nucleic acid formulations set forth herein. Patients may, but need not, have received previous chemo- radio- or gene therapeutic treatments. Optimally the patient may exhibit adequate bone marrow function (e.g., peripheral absolute granulocyte count of >2,000/mm3 and platelet count of 100, 000/mm3, adequate liver function (bilirubin 1.5 mg/dl) and adequate renal function (e.g., creatinine 1.5 mg/dl).

The nucleic acid formulation may be any of the formulations set forth herein, such as a formulation suitable for topical or oral administration. The formulation may include one or more therapeutic nucleic acids in dosage unit formulations containing any of the carriers, adjuvants, and vehicles as set forth above. The composition may be orally ingested or topically applied, such as using an applicator. Where a combination therapy is contemplated, the composition may be administered before, after or concurrently with the other anti-cancer agents.

In one example, a treatment course can comprise about six doses delivered over a 7 to 21 day period. Upon election by the clinician, the regimen may be continued six doses every three weeks or on a less frequent (monthly, bimonthly, quarterly etc.) basis. Of course, these are only exemplary times for treatment, and the skilled practitioner can readily recognize that many other time-courses are possible.

In some embodiment, administration may entail topical application of the nucleic acid composition on a skin or mucosal surface. In another embodiment, a catheter can be inserted into a postsurgical wound following tumor excision, and the cavity may be continuously perfused for a desired period of time.

Clinical responses can be defined by acceptable measures known to those of skill in the art. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites. Those of skill in the art can take the information disclosed in this specification and optimize the treatment regimen.

### Example 9

Clinical Trials of the Use of Nucleic Acid Formulations for Treatment of a Wound

Using the teachings of the specification and the knowledge of those skilled in the art, one can design protocols that can be used to facilitate the treatment of wounds in human subjects using one of the nucleic acid formulations set forth herein, such as a formulation that includes a nucleic acid encoding a growth factor. The wound, for example, may be a postsurgical wound (such as a wound following excision of a tumor), or a traumatic wound.

A composition of the present invention can be typically administered topically to the wound in dosage unit formulations containing carriers, adjuvants, and vehicles as set forth above. In certain instances, the formulation may include a nucleic acid encoding an anticancer agent, such as a tumor suppressor gene, in addition to the growth factor. Further, the therapeutic nucleic acid may or may not be administered in conjunction with other standard therapies of a wound, such as antibiotic therapy. Where a combination therapy is contemplated, the therapeutic nucleic acid can be administered before, after or concurrently with any secondary therapeutic agents. Where the wound is a surgical wound, therapy can be administered before, after, or concurrently with the surgical procedure.

For example, a treatment course can comprise about six doses delivered over a 1 to 6 day period. Upon election by the clinician the regimen may be continued at a more or less frequent basis. Of course, these are only exemplary times for treatment, and the skilled practitioner can readily recognize that many other time-courses are possible. Response to therapy will likely be a key factor in determining the dosage regimen.

In one embodiment, administration may simply entail topical application of the therapeutic composition to the wound. In another embodiment, a catheter can be inserted into the wound and the wound continuously perfused for a desired period of time.

Clinical responses can be defined by any acceptable measure known to those of skill in the art, such as visual inspection of the wound for signs of healing, such as decrease in wound size, decrease in inflammation, and so forth.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,348,502
U.S. Pat. No. 4,112,942
U.S. Pat. No. 4,321,251
U.S. Pat. No. 4,372,944
U.S. Pat. No. 4,415,723
U.S. Pat. No. 4,458,066
U.S. Pat. No. 4,469,863
U.S. Pat. No. 4,526,899
U.S. Pat. No. 4,627,979
U.S. Pat. No. 4,682,195
U.S. Pat. No. 4,683,202
U.S. Pat. No. 4,797,368
U.S. Pat. No. 4,868,168
U.S. Pat. No. 4,898,592
U.S. Pat. No. 4,919,918
U.S. Pat. No. 4,946,778
U.S. Pat. No. 5,023,243
U.S. Pat. No. 5,078,993
U.S. Pat. No. 5,139,941
U.S. Pat. No. 5,158,761
U.S. Pat. No. 5,194,269
U.S. Pat. No. 5,250,298
U.S. Pat. No. 5,264,618
U.S. Pat. No. 5,354,855
U.S. Pat. No. 5,372,801
U.S. Pat. No. 5,423,803
U.S. Pat. No. 5,456,918
U.S. Pat. No. 5,459,127
U.S. Pat. No. 5,470,561
U.S. Pat. No. 5,571,314
U.S. Pat. No. 5,599,712
U.S. Pat. No. 5,645,897
U.S. Pat. No. 5,652,194
U.S. Pat. No. 5,660,866
U.S. Pat. No. 5,672,344
U.S. Pat. No. 5,681,827
U.S. Pat. No. 5,695,746
U.S. Pat. No. 5,770,219
U.S. Pat. No. 5,783,208
U.S. Pat. No. 5,795,715
U.S. Pat. No. 5,830,499
U.S. Pat. No. 5,861,397
U.S. Pat. No. 5,874,094
U.S. Pat. No. 5,888,493
U.S. Pat. No. 5,888,773
U.S. Pat. No. 5,889,136
U.S. Pat. No. 5,914,334
U.S. Pat. No. 5,925,565
U.S. Pat. No. 5,928,906
U.S. Pat. No. 5,935,819
U.S. Pat. No. 5,993,785
U.S. Pat. No. 5,994,317
U.S. Pat. No. 6,024,733
U.S. Pat. No. 6,086,852
U.S. Pat. No. 6,117,417
U.S. Pat. No. 6,119,036
U.S. Pat. No. 6,140,355
U.S. Pat. No. 6,147,055
U.S. Pat. No. 6,165,494
U.S. Pat. No. 6,165,615
U.S. Pat. No. 6,166,004
U.S. Pat. No. 6,166,043
U.S. Pat. No. 6,166,084
U.S. Pat. No. 6,171,611
U.S. Pat. No. 6,194,388
U.S. Pat. No. 6,206,646
U.S. Pat. No. 6,207,646
U.S. Pat. No. 6,221,712
U.S. Pat. No. 6,251,370
U.S. Pat. No. 6,258,374
U.S. Pat. No. 6,258,830
U.S. Pat. No. 6,261,574
U.S. Pat. No. 6,267,984
U.S. Pat. No. 6,269,810
U.S. Pat. No. 6,273,884
U.S. Pat. No. 6,280,752
U.S. Pat. No. 6,280,766
U.S. Pat. No. 6,299,631
U.S. Pat. No. 6,309,387
U.S. Pat. No. 6,310,036
U.S. Pat. No. 6,329,337
U.S. Pat. No. 6,333,194
U.S. Pat. No. 6,339,068
U.S. Pat. No. 6,348,187
U.S. Pat. No. 6,348,450
U.S. Pat. No. 6,348,502
U.S. Pat. No. 6,387,352
U.S. Pat. No. 6,399,588
U.S. Pat. No. 6,503,481
U.S. Pat. No. 6,508,647
U.S. Pat. No. 6,551,578
U.S. Pat. No. 6,555,131
U.S. Pat. No. 6,555,376
U.S. Pat. No. 6,558,043
U.S. Pat. No. 6,588,043
U.S. Pat. No. 6,596,318
U.S. Pat. No. 6,610,272
U.S. Pat. No. 6,620,451
U.S. Pat. No. 6,629,091
U.S. Pat. No. 6,629,974
U.S. Pat. No. 6,670,332
U.S. Pat. No. 6,673,863
U.S. Pat. No. 6,705,316
U.S. Pat. No. 6,721,595
U.S. Pat. No. 6,723,114
U.S. Pat. No. 6,797,704
U.S. Pat. No. 6,828,308
U.S. Pat. No. 6,835,392
U.S. Pat. No. 6,841,539
U.S. Pat. No. 6,881,776
U.S. Pat. No. 6,887,856
U.S. Pat. No. 6,923,976
U.S. Pat. No. 6,939,859
U.S. Pat. No. 6,942,878
U.S. Pat. No. 6,946,144
U.S. Pat. No. 6,967,023
U.S. Pat. No. 6,982,091
U.S. Appln. 20020028785
U.S. Appln. 20020037323
U.S. Appln. 20020044910
U.S. Appln. 20020045148
U.S. Appln. 20020116026
U.S. Appln. 20030119985
U.S. Appln. 20030152530
U.S. Appln. 20040018155
U.S. Appln. 20040076590
U.S. Appln. 20040146919
U.S. Appln. 20040199207
U.S. Appln. 20040213764
U.S. Appln. 20050143336
U.S. Appln. 20050196343
U.S. Appln. 20050203047
U.S. Appln. 20050260276
U.S. application Ser. No. 09/351,778
Agrawal, et al., Proc. Natl. Acad. Sci. USA, 91:7595, 1991.
Akomeah et al., Eur. J. Pharm. Sci., 21(2-3):337-45, 2004.
Aksentijevich et al., Hum. Gene Ther., 7(9):1111-1122, 1996.
Alam and Cook, Anal. Biochem., 188:245-254, 1990.
Alauddin and Conti, Nucl. Med. Biol., 25(3):175-180 1998.
Alauddin et al., Nucl. Med. Biol., 23(6):787-792 1996.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Atiyeh et al., In: State of the Art in Burn Treatment, World J. Surgery, 2005.
Auricchio et al., Hum. Gene Ther., 14(3):255-261, 2003.
Banerji et al., Cell, 27(2 Pt 1):299-308, 1981.
Banerji et al., Cell, 33(3):729-740, 1983.
Bates et al., Nature, 395:124-125, 1998.
Batterson and Roizman, J. Virol., 46(2):371-377, 1983.
Baxter and Mitragotri, J. Control. Release, 106(3):361-73, 2005.
Bedi et al., Cancer Res., 56(11):2484-2487, 1996.
Berkhout et al., Cell, 59:273-282, 1989.
Bernstein et al., Nature, 409:363-366, 2001.
Bianco, Expert Opin. Drug Deliv., 1(1):57-65, 2004.
Blanar et al., EMBO J., 8:1139, 1989.
Blasberg, Mol. Cancer Ther., 2(3):335-343, 2003.
Blasberg, Nucl. Med. Biol., 30(8):879-888, 2003.
Block, In: Pharmaceutical Dosage Forms, Lieberman et al. (Eds.), Marcel Dekker, NY, 1: 335, 1988.
Blumberg et al., Cell, 104(1):9-19, 2001.
Bodine and Ley, EMBO J., 6:2997, 1987.
Bok and Bok, In: Manual of Wound Management and Healing, McGraw-Hill Professional, 1^{st} Ed., 2004.
Boshart et al., Cell, 41:521, 1985.
Bosze et al., EMBO J., 5(7):1615-1623, 1986.
Braddock et al., Cell, 58:269, 1989.
Bramson et al., Gene Ther., 10(3):251-60, 2003.
Brennecke et al., Cell, 113:25-36, 2003.
Brugmans et al., JAMA, 217:313-316, 1971.
Bulla and Siddiqui, J. Virol., 62:1437, 1986.
Burns et al., In: Dermatology—Rook's Textbook of Dermatology, Burns et al., (Eds.), Blackwell Publishers; 7th Ed., 2004.
Caley et al., J. Virology, 71(4):3031-3038, 1997.
Campbell and Villarreal, Mol. Cell. Biol., 8:1993, 1988.
Campere and Tilghman, Genes and Dev., 3:537, 1989.
Campo et al., Nature, 303:77, 1983.
Caplen et al., Gene, 252(1-2):95-105, 2000.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Hepatology, 14:134A, 1991.
Chang et al., Mol. Cell. Biol., 9:2153, 1989.
Chatterjee et al, Proc. Natl. Acad. Sci. USA, 86:9114, 1989.
Choi et al., Cell, 53:519, 1988.
Clark et al., Hum. Gene Ther., 6(10):1329-1341, 1995.
Clayman et al., Cancer Res., 55(14):1-6, 1995.
Cocea, Biotechniques, 23(5):814-816, 1997.
Coffin, In: Virology, Fields et al. (Eds.), Raven Press, NY, 1437-1500, 1990.
Cohen et al., J. Cell. Physiol., 5:75, 1987.
Cook et al., Cell, 27:487-496, 1981.
Costa et al., Mol. Cell. Biol., 8:81, 1988.
Couch et al., Am. Rev. Resp. Dis., 88:394-403, 1963.
Cowdery et al., J. Immunol., 156: 4570-4575, 1996
Cripe et al., EMBO J., 6:3745, 1987.
Culotta and Hamer, Mol. Cell. Biol., 9:1376, 1989.
Dalgleish, Expert Rev. Vaccines, 3(6):665-668, 2004.
Dandolo et al., J. Virology, 47:55-64, 1983.
Davis et al, Curr. Biol., 6:146-148, 1996.
De Villiers et al., Nature, 312(5991):242-246, 1984.
de Waal et al., J. Exp. Med., 174:1209-1220, 1991.
DeLuca et al., J. Virol., 56(2):558-570, 1985.
Deschamps et al., Science, 230:1174-1177, 1985.
Devulapalle and Mooser, J. Dent. Res., 80(2):466-469, 2001.
Doench et al., Genes Dev., 17:438-42, 2003.
Doerr, Nat. Methods, 2(11):808, 2005.
Doukas et al., Hum Gene Ther., 12(7):783-98, 2001.
Du and Zamore, Development, 132(21):4645-4652, 2005.
Dubey et al., Proc. Natl. Acad. Sci. USA, 100(3):1232-1237, 2003.
Dumon et al., Proc. Natl. Acad. Sci. USA, 98(6):3346-3351 2001.
Dumoutier et al., J. Immunol., 164(4):1814-1819., 2000.
Edbrooke et al., Mol. Cell. Biol., 9:1908, 1989.
Edlund et al., Science, 230:912-916, 1985.
Eicher et al., Clin. Cancer Res., 2(10):1659-1664, 1996.
Ekmekcioglu et al., Int. J. Cancer, 94(1):54-59, 2001.
Ekmekciouglu et al., Melanoma Res., 9(3):261-272, 1999.
Elbashir et al., EMBO, 20(23):6877-6888, 2001.
Elbashir et al., EMBO, 20(23):6877-6888, 2001.
Elbashir et al., Genes Dev., 5(2):188-200, 2001.
Elbashir et al., Genes Dev., 5(2):188-200, 2001.
Elroy-Stein et al., Proc. Natl. Acad. Sci. USA, 86(16):6126-6130, 1989.
Eriksson and Vranckx, Am. J. Surg., 188(1A Suppl):36-41; 2004.
Feng and Holland, Nature, 334:6178, 1988.
Ferguson and O'Kane, Biol. Sci., 359(1445):839-850, 2004.
Finn et al., Cancer Chemother. Biol. Response Modif., 21:223-233, 2003.
Finn, Nat. Rev. Immunol., 3(8):630-641, 2003.
Firak and Subramanian, Mol. Cell. Biol., 6:3667, 1986.
Fire et al., Nature, 391(6669):806-811, 1998.
Flotte and Carter, Gene Ther., 2(6):357-62, 1995.
Flotte et al., Am. J. Respir. Cell Mol. Biol., 7(3):349-356, 1992.
Flotte et al., Proc. Natl. Acad. Sci. USA, 90(22):10613-10617, 1993.
Foecking and Hofstetter, Gene, 45(1):101-105, 1986.
Forster and Symons, Cell, 49(2):211-220, 1987.
Fraley et al., Proc. Natl. Acad. Sci. USA, 76:3348-3352, 1979.
Friedman and Platzer, Biochim. Biophys. Acta, 544:605-614, 1978.
Friedman and Platzer, Biochim. Biophys. Acta, 630:271-278, 1980.
Fujita et al., Cell, 49:357, 1987.
Gabizon et al., Cancer Res., 50(19):6371-6378, 1990.
Gallichan et al., J Infect Dis., 168(3):622-629, 1993.

Gallichan and Rosenthal, J. Exp. Med., 194(5):1879-1990, 1996.
Gallichan and Rosenthal, Vaccine, 13(16):1589-1595, 1995.
Gallagher et al., Genes Immun., 1(7):442-450, 2000.
Gambhir et al., J. Nucl. Med., 39(11):2003-2011, 1998.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Glorioso et al., Mol. Biotechnol., 4(1):87-99, 1995.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodchild, Bioconjugate Chem., 1: 165-187, 1990.
Gottschall et al., Endocrinology, 127(1):272-277, 1990.
Graham and Prevec, In: Methods in Molecular Biology: Gene Transfer and Expression Protocol, Murray (Ed.), Humana Press, NJ, 7:109-128, 1991.
Graham and Van Der Eb, Virology, 52:456-467, 1973.
Graham et al., J. Gen. Virl., 36(1):59-74, 1977.
Greene et al., Immunology Today, 10:272, 1989
Grishok et al., Cell, 106:23-34, 2001.
Grishok et al., Science, 287:2494-2497, 2000.
Grosschedl and Baltimore, Cell, 41:885, 1985.
Grunhaus and Horwitz, Seminar in Virology, 3:237-252, 1992.
Gu et al., Mol. Ther., 9(5):699-711, 2004.
Guo et al., J. Dent. Res., 83(3):266-270, 2004.
Halpern et al., Cell Immunol., 167(1):72-78, 1996.
Haslinger and Karin, Proc. Natl. Acad. Sci. USA, 82:8572, 1985.
Hauber and Cullen, J. Virology, 62:673, 1988.
Hellstrom and Hellstrom, Expert Rev. Vaccines, 2(4):517-532, 2003.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Higuchi et al., Remington's Pharmaceutical Sciences, Mack Publishing Co., Easton, Pa., 301, 1985.
Hirochika et al., J. Virol., 61:2599, 1987.
Hirsch et al., Mol. Cell. Biol., 10:1959, 1990.
Holbrook et al., Virology, 157:211, 1987.
Holland and Holland, J Biol Chem, 255(6):2596-605, 1980.
Honess and Roizman, J. Virol., 14(1):8-19, 1974.
Honess and Roizman, J. Virol., 16(5):1308-130826. 1975.
Hooper et al., J. Biolumin. Chemilumin., 5(2):123-130, 1990.
Horlick and Benfield, Mol. Cell. Biol., 9:2396, 1989.
Horwich, et al., J. Virol., 64:642-650, 1990.
Huang et al., Cell, 27:245, 1981.
Hug et al., Mol. Cell. Biol., 8:3065, 1988.
Hutvagner and Zamore, Science, 297(5589):2056-2060, 2002.
Hutvagner et al., Science, 293:834-838, 2001.
Hwang et al., Mol. Cell. Biol., 10:585, 1990.
Idson, In: Pharmaceutical Dosage Forms, Lieberman (Eds.), 1:199, 1988.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jiang et al., Nature, 375:151-155, 1995.
Jiang et al., Oncogene, 11(12):2477-2486, 1995.
Jiang et al., Proc. Natl. Acad. Sci. USA, 93(17):9160-9165, 1996.
Johnson et al., Mol. Cell. Biol., 9:3393, 1989.
Jones and Shenk, Cell, 13:181-188, 1978.
Joyce, Nature, 338:217-244, 1989.
Kadesch and Berg, Mol. Cell. Biol., 6:2593, 1986.
Kaneda et al., Science, 243:375-378, 1989.
Kaplitt et al., Nat. Genet., 8(2):148-154, 1994.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamoto et al., Mol. Cell. Biol., 8:267, 1988.
Ketting et al., Cell, 99(2):133-141, 1999.
Ketting et al., Genes Dev., 15(20):2654-2659, 2001.
Khuri et al., J. Natl. Cancer Inst., 89(3):199-211, 1997.
Kiledjian et al., Mol. Cell. Biol., 8:145, 1988.
Kim and Cook, Proc. Natl. Acad. Sci. USA, 84(24):8788-8792, 1987.
Klamut et al., Mol. Cell. Biol., 10: 193, 1990.
Klinman, et al., Proc. Natl. Acad. Sci. USA, 93(7):2879-2883, 1996.
Knapp et al., Atherosclerosis, 152(1):217-227, 2000.
Knight and Bass, Science, 2:2, 2001.
Koch et al., Mol. Cell. Biol., 9:303, 1989.
Koehne et al., Nat. Biotechnol., 21(4):405-413, 2003.
Kohler and Bachmann, Mol. Biochem. Parasitol., 4(5-6):325-336, 1981.
Kornberg and Baker, DNA Replication, 2nd Ed., Freeman, San Francisco, 1992.
Kotenko et al., Proc. Natl. Acad. Sci. USA, 97(4):1695-1700, 2000.
Kotin et al., Proc. Natl. Acad. Sci. USA, 87(6):2211-2215, 1990.
Krieg et al., Nature, 374:546-549, 1995.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kundra et al., J. Nucl. Med., 43(3):406-412, 2002.
Kundra et al., Q J Nucl. Med. Mol. Imaging, 49(4):325-338.2005.
Kunz et al., Nucl. Acids Res., 17:1121, 1989.
Lacey and Watson, Biochem. Pharmacol., 34:1073-1077, 1989.
Lacey, Int. J. Parasitol., 18(7):885-936, 1988.
LaFace et al., Virology, 162(2):483-486, 1988.
Larsen et al., Proc Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Levrero et al., Gene, 101: 195-202, 1991.
Liang et al., Nucleic Acids Res., 3:33(19):e170, 2005.
Lin and Avery, Nature, 402:128-129, 1999.
Lin et al., Mol. Cell. Biol., 10:850, 1990.
Linchey and Fridovich, Free Radic. Biol. Med., 23(4):668-671, 1997.
Liu et al., Cancer Res., 55(14):3117-3122, 1995.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Lydiatt et al., Cancer, 82(7):1376-1380, 1998.
Macejak and Samow, Nature, 353:90-94, 1991.
MacLaren et al., Gene Ther., 6:785-791, 1999.
Majors and Varmus, Proc. Natl. Acad. Sci. USA, 80:5866, 1983.
Mann et al., Cell, 33:153-159, 1983.
Markowitz et al., J. Virol., 62:1120-1124, 1988.
McCarty et al., J. Virol., 65(6):2936-2945, 1991.
McGrory et al., Virology, 163(2):614-617, 1988.
McLaughlin et al., J. Virol., 62(6):1963-1973, 1988.
McNeall et al., Gene, 76:81, 1989.
Messina et al., J. Immunol., 147(6):1759-1764 1991.
Michel and Westhof, J. Mol. Biol., 216:585-610, 1990.
Michiels et al., Arch. Int. Pharmacodyn Ther., 256(2):180-191, 1982.
Miksicek et al., Cell, 46:203, 1986.
Montgomery et al., Proc. Natl. Acad. Sci. USA, 95:15502-15507, 1998.
Mordacq and Linzer, Genes and Dev., 3:760, 1989.
Moreau et al., Nuc. Acids Res., 9:6047, 1981.
Mourelatos et al., Genes Dev., 16(6):720-728, 2002.
Muesing et al., Cell, 48:691, 1987.
Mukhopadhyay et al., Clin. Cancer Res., 8(9):2963-2969, 2002.
Muzyczka, Curr. Topics Microbiol. Immunol., 158:97-129, 1992.
Neville et al., In: Dentistry—Oral & Maxillofacial Pathology, W B Saunders; 2nd Ed., 2001.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ohi et al., Gene, 89(2):279-282, 1990.
Ondek et al., EMBO J., 6:1017, 1987.
Ornitz et al., Mol. Cell. Biol., 7:3466, 1987.
Palmiter et al., Nature, 300:611, 1982.
Paskind et al., Virology, 67:242-248, 1975.
PCT Appln. WO 00/44914
PCT Appln. WO 01/36646
PCT Appln. WO 01/68836
PCT Appln. WO 0208436
PCT Appln. WO 0231168
PCT Appln. WO 0285287
PCT Appln. WO 0333029
PCT Appln. WO 98/07408
PCT Appln. WO 99/32619
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Picard and Schaffner, Nature, 307:83, 1984.
Pinkert et al., Genes and Dev., 1:268, 1987.
Plotkin et al., In: Vaccines, W.B. Saunders Company, 4^{th} Ed., 2003.
Ponta et al., Proc. Natl. Acad. Sci. USA, 82:1020, 1985.
Porton et al., Mol. Cell. Biol., 10: 1076, 1990.
Post et al., Cell, 24(2):555-65, 1981.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reinhart et al., Nature, 403:901-906, 2000.
Reinhold-Hurek and Shub, Nature, 357:173-176, 1992.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ripe et al., Mol. Cell. Biol., 9:2224, 1989.
Rittling et al., Nuc. Acids Res., 17:1619, 1989.
Robinson et al., In: Vaccine Protocols (Methods in Molecular Medicine), Humana Press, 2^{nd} Ed., 2003.
Rosen et al., Cell, 41:813, 1988.
Rosenberg et al., Science, 223:218-1321, 1986.
Rosenfeld et al., Science, 252:431-434, 1991.
Rosenfeld, et al., Cell, 68:143-155, 1992.
Rosoff, In: Pharmaceutical Dosage Forms: Tablets, 2^{nd} Ed., 1:245, Lieberman et al. (Eds.), 1989.
Roux et al., Proc. Natl. Acad. Sci. USA, 86:9079-9083, 1989.
Russell et al., Biochem Pharmacol., 43(5): 1095-1100, 1992.
Sakai et al., Genes and Dev., 2:1144, 1988.
Sambrook et al., In: Molecular cloning, Cold Spring Harbor Laboratory Press, Cold Spring Harbor, N.Y., 2001.
Samulski et al., J. Virol., 63:3822-3828, 1989.
Sarver et al., Science, 247:1222-1225, 1990.
Sasaki et al., Mol. Cancer. Ther., 1(13):1201-1209, 2002.
Satake et al., J. Virology, 62:970, 1988.
Saxena et al, J. Biol. Chem., 278(45):44312-44319, 2003.
Scanlon et al., Proc. Natl. Acad. Sci. USA, 88:10591-10595, 1991.
Schaefer et al., Cell Signal, 12(3):143-151, 2000.
Schaffner et al., J. Mol. Biol., 201:81, 1988.
Scheit, In: Synthesis and Biological Function, Wiley-Interscience, NY, 171-172, 1980.
Searle et al., Mol. Cell. Biol., 5:1480, 1985.
Sharp and Marciniak, Cell, 59:229, 1989.
Sharp and Zamore, Science, 287:2431-2433, 2000.
Sharp, Genes Dev., 13:139-141, 1999.
Shaul and Ben-Levy, EMBO J., 6:1913, 1987.
Shelling and Smith, Gene Therapy, 1:165-169, 1994.
Sherman et al., Mol. Cell. Biol., 9:50, 1989.
Siegfried, Exp. Clin. Endocrinol., 101(1):7-11, 1993.
Simon et al., BMJ, 328(7452):1358-1362, 2004.
Sleigh and Lockett, J. EMBO, 4:3831, 1985.
Smith and Moss, Gene, 25(1):21-8, 1983.
Solodin et al., Biochemistry, 34(41):13537-13544, 1995.
Soo et al., J. Cell. Biochem., 74(1):1-10, 1999.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Straus et al., J. Cell Biol., 99(5):1838-1847, 1984.
Stuart et al., Nature, 317:828, 1985.
Sullivan and Peterlin, Mol. Cell. Biol., 7:3315, 1987.
Sun et al., Gene Ther., 8:1572-1579, 2001.
Suresh et al., Exp. Hematology, 21: 1828 1993.
Swartzendruber and Lehman, J. Cell. Physiology, 85:179, 1975.
Tabara et al., Cell, 99(2):123-132, 1999.
Takebe et al., Mol. Cell. Biol., 8:466, 1988.
Tang et al., Blood, 104(9):2704-2713, 2004.
Tarnawski, Dig. Dis. Sci., 50 Suppl 1:S24-33, 2005.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thierry et al., Proc. Natl. Acad. Sci. USA, 92(21):9742-9746, 1995.
Thiesen et al., J. Virology, 62:614, 1988.
Tjuvajev et al., Cancer Res., 58:4333-4341, 1998.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Treisman, Cell, 42:889, 1985.
Tronche et al., Mol. Biol. Med., 7:173, 1990.
Trudel and Constantini, Genes and Dev., 6:954, 1987.
Tsukamoto et al., Nat. Genet., 9(3):243-248, 1995.
Tuqan et al., Lasers Med. Sci., 20(2):80-6, 2005.
Tyndell et al., Nuc. Acids. Res., 9:6231, 1981.
Uhlmann and Peyman, Chem. Rev., 90:544-584, 1990.
Umeoka et al, Cancer Res., 64:6259-6265, 2004.
Van den Bossche et al., Chemother., 19:67-128, 1982.
Van den Bossche, Ann. Soc. Belg. Med. Trop., 61:287-296, 1981.
Vannice and Levinson, J. Virology, 62:1305, 1988.
Vasseur et al., Proc Natl. Acad. Sci. USA, 77:1068, 1980.
Vogelstein et al., Nature, 408(6810):307-310, 2000.
Wailer et al., Microsurgery, 24(3):168-173, 2004.
Walsh et al., J. Clin. Invest, 94:1440-1448, 1994.
Wang and Calame, Cell, 47:241, 1986.
Watts et al., Biochem. Pharmacol., 31:3035-3040, 1982.
Weber et al., Cell, 36:983, 1984.
Wei et al., Gene Therapy, 1:261-268, 1994.
Weinberger et al. Mol. Cell. Biol., 8:988, 1984.
Wheeler et al., Gastrointerology, 120:1241-1250, 2001
Wheeler et al., Hum. Gene Ther., 12:2167-2177, 2001
Wincott et al., Nucleic Acids Res., 23(14):2677-2684, 1995.
Winoto and Baltimore, Cell, 59:649, 1989.
Witassek et al., Eur. J. Clin. Pharmacol., 20:427-433, 1981.
Wong et al., Gene, 10:87-94, 1980.
Xu et al., Curr. Biol., 13:790-795, 2003.
Yamamoto et al., J. Immunol., 148: 4072-4076, 1992.
Yang and Huang, Gene Therapy, 4 (9):950-960, 1997.
Yang et al., J. Virol., 68:4847-4856, 1994.
Yang et al., Radiology, 235(3):950-958, 2005.
Yanoff et al., In: Opthalmology—Ophthalmology, 2^{ND} Bk&Cdr Ed., 2003.
Yoder et al., Blood, 82 (Supp.): 1:347A, 1994.
Yutzey et al. Mol. Cell. Biol., 9:1397, 1989.
Zeng et al., Cancer Res., 62(13):3630-3635, 2002.
Zhang et al., Endocrinology, 141:4698-4710, 2000.
Zhou et al., Exp. Hematol, 21:928-933, 1993.
Zhou et al., J. Exp. Med., 179:1867-1875, 1994.
Zhu et al., Science, 261(5118):209-211, 1993.

1. A pharmaceutical composition comprising a therapeutic nucleic acid and/or a diagnostic nucleic acid, wherein the composition is formulated as a lozenge, a lollipop, a popsicle, a gum, a gel strip, a film, a hydrogel, a dissolving strip, or a solid stick.

2. The pharmaceutical composition of claim 1, wherein the composition comprises a therapeutic nucleic acid.

3. (canceled)

4. (canceled)

5. The pharmaceutical composition of claim 1, wherein the composition comprises a diagnostic nucleic acid that encodes a reporter protein.

6. (canceled)

7. (canceled)

8. The pharmaceutical composition of claim 1, wherein the formulation further comprises collagen, glycerin, PEG, hydrated silica, cellulose, xanthum gum, glycan carbomer 956, Tween 80, fluoride, Carrageenan, an adhesive or a nucleic acid uptake enhancer.

9. The pharmaceutical composition of claim 8, wherein the adhesive comprises an acrylate, a hydrocolloid, a hydrogel, a polyacrylic acid-based gel matrix, a polyisobutylene, a silicone polymer, or a mixture thereof.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The pharmaceutical composition of claim 27, wherein the expression cassette is carried in a viral vector.

29. (canceled)

30. (canceled)

31. (canceled)

32. (canceled)

33. (canceled)

34. (canceled)

35. (canceled)

36. (canceled)

37. The pharmaceutical composition of claim 1, wherein the composition further comprises a delivery agent.

38. The pharmaceutical composition of claim 37, wherein the delivery agent is a lipid.

39. (canceled)

40. (canceled)

41. (canceled)

42. (canceled)

43. (canceled)

44. (canceled)

45. (canceled)

46. (canceled)

47. A non-adenoviral pharmaceutical composition comprising a therapeutic nucleic acid and/or a diagnostic nucleic acid, wherein the composition is formulated as a gel, a paste, a foam, a slurry, a cream, a salve, a suppository, or a powder.

48. (canceled)

49. The pharmaceutical composition of claim 48, wherein the expression cassette is carried in a viral vector.

50. (canceled)

51. (canceled)

52. The pharmaceutical composition of claim 47, wherein the composition comprises a therapeutic nucleic acid that encodes p53.

53. The pharmaceutical composition of claim 47, wherein the composition comprises a therapeutic nucleic acid that encodes mda7.

54. (canceled)

55. The pharmaceutical composition of claim 47, wherein the composition comprises a therapeutic nucleic acid that encodes FUS1.

56. (canceled)

57. The pharmaceutical composition of claim 47, wherein the paste is further defined as a toothpaste.

58. A pharmaceutical composition comprising a therapeutic and/or diagnostic nucleic acid and an adhesive.

59. The pharmaceutical composition of claim 58, wherein the composition comprises a therapeutic nucleic acid.

60. (canceled)

61. (canceled)

62. (canceled)

63. (canceled)

64. (canceled)

65. The pharmaceutical composition of claim 58, wherein the nucleic acid is a diagnostic nucleic acid that encodes a fluorescent protein.

66. (canceled)

67. The pharmaceutical composition of claim 58, wherein the nucleic acid is comprised in an expression cassette comprising a promoter operatively coupled to the nucleic acid, wherein the promoter is active in cells of a subject.

68. The pharmaceutical composition of claim 67, wherein the expression cassette is carried in a viral vector.

69. (canceled)

70. The pharmaceutical composition of claim 68, wherein the viral vector is an adenoviral vector.

71. The pharmaceutical composition of claim 70, wherein the composition comprises a therapeutic nucleic acid that encodes p53.

72. The pharmaceutical composition of claim 70, wherein the composition comprises a therapeutic nucleic acid that encodes mda7.

73. The pharmaceutical composition of claim 70, wherein the composition comprises a therapeutic nucleic acid that encodes FUS1.

74. (canceled)

75. (canceled)

76. The pharmaceutical composition of claim 67, wherein the expression cassette is carried in a delivery agent.

77. The pharmaceutical composition of claim 76, wherein the delivery agent is a lipid.

78. (canceled)

79. (canceled)

80. (canceled)

81. (canceled)

82. (canceled)

83. The pharmaceutical composition of claim 58, wherein the adhesive comprises an acrylate, a hydrocolloid, a hydrogel, a polyacrylic acid-based gel matrix, a polyisobutylene, a silicone polymer, or a mixture thereof

84. (canceled)

85. The pharmaceutical composition of claim 58, wherein the composition is formulated to be administered via a transdermal patch, a strip, a bandage, a tape, a dressing, or a synthetic skin.

86. The pharmaceutical composition of claim 58, wherein the composition is formulated as a liquid, a semi-solid, or a solid.

87. (canceled)

88. (canceled)

89. (canceled)

90. A transdermal or transcutaneous delivery device for delivery of a therapeutic or diagnostic agent to a subject, comprising:
a) a patch; and
b) a pharmaceutical composition comprising a nucleic acid encoding a reporter protein, a tumor suppressor, a pro-apoptotic protein, a growth factor, a tumor antigen, or a cytokine applied to at least one surface of the patch.

91. (canceled)

92. (canceled)

93. (canceled)

94. (canceled)

95. (canceled)

96. The device of claim 90, wherein the nucleic acid is comprised in an expression cassette comprising a promoter operatively coupled to the nucleic acid, wherein the promoter is active in cells of a subject.

97. The device of claim 96, wherein the expression cassette is carried in a viral vector.

98. (canceled)

99. (canceled)

100. (canceled)

101. (canceled)

102. (canceled)

103. (canceled)

104. (canceled)

105. (canceled)

106. (canceled)

107. A method of detecting, treating, or preventing disease in a subject, comprising administering to the subject a pharmaceutical composition comprising a therapeutic nucleic acid and/or a diagnostic nucleic acid, wherein the composition is formulated as a lozenge, a lollipop, a popsicle, a gum, a gel strip, a film, a hydrogel, a dissolving strip, or a solid stick or a pharmaceutical composition comprising a therapeutic and/or diagnostic nucleic acid and an adhesive.

108. The method of claim 107, wherein the nucleic acid encodes a reporter protein, and wherein the method is further defined as a method of detecting a lesion in a subject.

109. The method of claim 108, wherein the lesion is a hyperproliferative lesion.

110. (canceled)

111. (canceled)

112. The method of claim 107, wherein the nucleic acid is a therapeutic nucleic acid.

113. (canceled)

114. (canceled)

115. (canceled)

116. (canceled)

117. (canceled)

118. (canceled)

119. The method of claim 112, wherein the method is further defined as a method of inducing an immune response in a mucosal surface, and wherein the pharmaceutical composition is applied to a mucosal surface of the subject.

120. The method of claim 107, wherein the composition comprises a diagnostic nucleic acid that encodes a reporter protein.

121. (canceled)

122. (canceled)

123. (canceled)

124. (canceled)

125. (canceled)

126. (canceled)

127. (canceled)

128. The method of claim 107, wherein the nucleic acid is comprised in an expression cassette comprising a promoter operatively coupled to the nucleic acid, wherein the promoter is active in cells of the subject.

129. The method of claim 128, wherein the expression cassette is carried in a viral vector.

130. (canceled)

131. (canceled)

132. (canceled)

133. (canceled)

134. (canceled)

135. The method of claim 107, wherein the subject is a mammal.

136. The method of claim 135, wherein the mammal is a human.

137. (canceled)

138. (canceled)

139. The method of claim 107, further comprising identifying a subject in need of detection, prevention, or treatment of a disease.

140. The method of claim 139, wherein the nucleic acid is a therapeutic nucleic acid, and wherein the method further comprises administration of one or more secondary forms of therapy to the subject.

141. A method of detecting, treating, or preventing disease in a subject, comprising administering to the subject a pharmaceutical composition as set forth in claim 47.

142. The method of claim 141, wherein the nucleic acid encodes a reporter protein, and wherein the method is further defined as a method of detecting a lesion in a subject.

143. The method of claim 142, wherein the lesion is a hyperproliferative lesion.

144. (canceled)

145. (canceled)

146. The method of claim 141, wherein the nucleic acid is a therapeutic nucleic acid.

147. (canceled)

148. (canceled)

149. (canceled)

150. (canceled)

151. (canceled)

152. (canceled)

153. (canceled)

154. The method of claim 141, wherein the composition comprises a diagnostic nucleic acid that encodes a reporter protein.

155. (canceled)

156. (canceled)

157. (canceled)

158. (canceled)

159. (canceled)

160. (canceled)

161. The method of claim 141, wherein the nucleic acid is comprised in an expression cassette comprising a promoter operatively coupled to the nucleic acid, wherein the promoter is active in cells of the subject.

162. The method of claim 161, wherein the expression cassette is carried in a viral vector.

163. (canceled)

164. (canceled)

165. (canceled)

166. (canceled)

167. The method of claim 141, wherein the subject is a mammal.

168. The method of claim 167, wherein the mammal is a human.

169. (canceled)

170. (canceled)

171. The method of claim 141, further comprising identifying a subject in need of detection, prevention, or treatment of a disease.

172. The method of claim 141, wherein the nucleic acid is a therapeutic nucleic acid, and wherein the method further comprises administration of one or more secondary forms of therapy to the subject.

173. A method of detecting, treating, or preventing disease in a subject, comprising applying to a surface of the subject a transdermal or transcutaneous delivery device as set forth in claim 90.

174. The method of claim 173, wherein the expression cassette is carried in a viral vector.

175. (canceled)

176. (canceled)

177. (canceled)

178. (canceled)

179. (canceled)


SEQUENCE LISTING (7 sequences)

SEQ ID NO: 1 - DNA, 19 bp, Artificial Sequence
  Description of Artificial Sequence  Synthetic Primer
ggcccacccccttggcttc

SEQ ID NO: 2 - DNA, 20 bp, Artificial Sequence
  Description of Artificial Sequence  Synthetic Primer
ttgtaaccattataagctgc

SEQ ID NO: 3 - DNA, 20 bp, Artificial Sequence
  Description of Artificial Sequence  Synthetic Primer
tcgtttctcagcagctgttg

SEQ ID NO: 4 - DNA, 20 bp, Artificial Sequence
  Description of Artificial Sequence  Synthetic Primer
catctgaactcaaagcgtgg

SEQ ID NO: 5 - DNA, 20 bp, Artificial Sequence
  Description of Artificial Sequence  Synthetic Primer
ttatgcgacggatcccgtaa

SEQ ID NO: 6 - DNA, 20 bp, Artificial Sequence
  Description of Artificial Sequence  Synthetic Primer
gcgtgtcaccgtcgtacgta

SEQ ID NO: 7 - DNA, 18 bp, Artificial Sequence
  Description of Artificial Sequence  Synthetic Primer
cttcgaggtccgcggccg